SEQUENCE LISTING (64 sequences)

SEQ ID NO: 1 - DNA, 20 bp, synthetic construct
  source 1..20: mol_type = other DNA
ggtagaagagaggcaaccga

SEQ ID NO: 2 - DNA, 16 bp, synthetic construct
  source 1..16: mol_type = other DNA
acccatggcggtgacc

SEQ ID NO: 3 - DNA, 17 bp, synthetic construct
  source 1..17: mol_type = other DNA
agccctgctcgatgctc

SEQ ID NO: 4 - DNA, 20 bp, synthetic construct
  source 1..20: mol_type = other DNA
ggctgcaggaatagggacaa

SEQ ID NO: 5 - DNA, 24 bp, synthetic construct
  source 1..24: mol_type = other DNA
caccggccctgtgagcatcgagca

SEQ ID NO: 6 - DNA, 24 bp, synthetic construct
  source 1..24: mol_type = other DNA
aaactgctcgatgctcacagggcc

SEQ ID NO: 7 - DNA, 23 bp, synthetic construct
  source 1..23: mol_type = other DNA
ctagcggaagattttatggcacc

SEQ ID NO: 8 - DNA, 20 bp, synthetic construct
  source 1..20: mol_type = other DNA
catatcgattggcccgggat

SEQ ID NO: 9 - DNA, 19 bp, synthetic construct
  source 1..19: mol_type = other DNA
cacgtctccgttctccgag

SEQ ID NO: 10 - DNA, 21 bp, synthetic construct
  source 1..21: mol_type = other DNA
ctgttgctgagagcattgaca

SEQ ID NO: 11 - DNA, 20 bp, synthetic construct
  source 1..20: mol_type = other DNA
cgagagctacacgttcacgg

SEQ ID NO: 12 - DNA, 21 bp, synthetic construct
  source 1..21: mol_type = other DNA
gggtgtcgagggaaaaatagg

SEQ ID NO: 13 - DNA, 23 bp, synthetic construct
  source 1..23: mol_type = other DNA
tagggaacaaggaatttttcggg

SEQ ID NO: 14 - DNA, 22 bp, synthetic construct
  source 1..22: mol_type = other DNA
gtacactaaccttcggtcaacc

SEQ ID NO: 15 - DNA, 20 bp, synthetic construct
  source 1..20: mol_type = other DNA
cggcactcagctacgctaac

SEQ ID NO: 16 - DNA, 21 bp, synthetic construct
  source 1..21: mol_type = other DNA
caagtccgggttggagtagac

SEQ ID NO: 17 - DNA, 21 bp, synthetic construct
  source 1..21: mol_type = other DNA
gtcacagtctcagtcacctgg

SEQ ID NO: 18 - DNA, 22 bp, synthetic construct
  source 1..22: mol_type = other DNA
gggataagacggatagtcctgc

SEQ ID NO: 19 - DNA, 20 bp, synthetic construct
  source 1..20: mol_type = other DNA
cccagcatgtgtgaccgaaa

SEQ ID NO: 20 - DNA, 21 bp, synthetic construct
  source 1..21: mol_type = other DNA
atcacgcaaggcttgatgtct

SEQ ID NO: 21 - DNA, 21 bp, synthetic construct
  source 1..21: mol_type = other DNA
cccatcaccatcttccaggag

SEQ ID NO: 22 - DNA, 22 bp, synthetic construct
  source 1..22: mol_type = other DNA
cttctccatggtggtgaagacg

SEQ ID NO: 23 - DNA, 19 bp, synthetic construct
  source 1..19: mol_type = other DNA
gcccctcattaagcccaag

SEQ ID NO: 24 - DNA, 21 bp, synthetic construct
  source 1..21: mol_type = other DNA
ttgtggtggtctgacagttcg

SEQ ID NO: 25 - DNA, 22 bp, synthetic construct
  source 1..22: mol_type = other DNA
tcagctgctgtttctctttcca

SEQ ID NO: 26 - DNA, 18 bp, synthetic construct
  source 1..18: mol_type = other DNA
gcggcgcatcttcttgtt

SEQ ID NO: 27 - DNA, 23 bp, synthetic construct
  source 1..23: mol_type = other DNA
tgccaagttgtacttactacgtc

SEQ ID NO: 28 - DNA, 23 bp, synthetic construct
  source 1..23: mol_type = other DNA
gttggaggagtaggagtatgtca

SEQ ID NO: 29 - DNA, 20 bp, synthetic construct
  source 1..20: mol_type = other DNA
aacttccggatctacccctg

SEQ ID NO: 30 - DNA, 20 bp, synthetic construct
  source 1..20: mol_type = other DNA
ctttctccagctccagggtc

SEQ ID NO: 31 - DNA, 21 bp, synthetic construct
  source 1..21: mol_type = other DNA
cctggaccgctttctcttgaa

SEQ ID NO: 32 - DNA, 21 bp, synthetic construct
  source 1..21: mol_type = other DNA
accgaaacaccggaagaagtc

SEQ ID NO: 33 - DNA, 20 bp, synthetic construct
  source 1..20: mol_type = other DNA
ggcgtcagagtgggaaatcc

SEQ ID NO: 34 - DNA, 21 bp, synthetic construct
  source 1..21: mol_type = other DNA
ggcaggcagttcacatctacc

SEQ ID NO: 35 - DNA, 21 bp, synthetic construct
  source 1..21: mol_type = other DNA
tgatttgtgggcctgaagaaa

SEQ ID NO: 36 - DNA, 21 bp, synthetic construct
  source 1..21: mol_type = other DNA
gaggcatctcagcagaagaca

SEQ ID NO: 37 - DNA, 20 bp, synthetic construct
  source 1..20: mol_type = other DNA
cccatttcggtagttgcagt

SEQ ID NO: 38 - DNA, 19 bp, synthetic construct
  source 1..19: mol_type = other DNA
ccttcagctaaagccccag

SEQ ID NO: 39 - DNA, 21 bp, synthetic construct
  source 1..21: mol_type = other DNA
tgtcagcaaaatcctgggcag

SEQ ID NO: 40 - DNA, 22 bp, synthetic construct
  source 1..22: mol_type = other DNA
gtcgggttctgtcgtttgtatt

SEQ ID NO: 41 - DNA, 21 bp, synthetic construct
  source 1..21: mol_type = other DNA
aagtgcagcaaccattcaacc

SEQ ID NO: 42 - DNA, 22 bp, synthetic construct
  source 1..22: mol_type = other DNA
ctgctctgtgagtcaatgctta

SEQ ID NO: 43 - DNA, 20 bp, synthetic construct
  source 1..20: mol_type = other DNA
acacggctgcatgagaacaa

SEQ ID NO: 44 - DNA, 19 bp, synthetic construct
  source 1..19: mol_type = other DNA
gccctcacgaagggatgtg

SEQ ID NO: 45 - DNA, 22 bp, synthetic construct
  source 1..22: mol_type = other DNA
agacaaccacaatggcttaaca

SEQ ID NO: 46 - DNA, 22 bp, synthetic construct
  source 1..22: mol_type = other DNA
tgaggctacatagtctcatggc

SEQ ID NO: 47 - DNA, 21 bp, synthetic construct
  source 1..21: mol_type = other DNA
gcacccggagagaagatcaaa

SEQ ID NO: 48 - DNA, 21 bp, synthetic construct
  source 1..21: mol_type = other DNA
taccaccgcatcagctcttta

SEQ ID NO: 49 - DNA, 20 bp, synthetic construct
  source 1..20: mol_type = other DNA
aaggcacactacatcgaggc

SEQ ID NO: 50 - DNA, 19 bp, synthetic construct
  source 1..19: mol_type = other DNA
cacgctgcgactcttttcc

SEQ ID NO: 51 - DNA, 19 bp, synthetic construct
  source 1..19: mol_type = other DNA
gtggaccgcacggaatttg

SEQ ID NO: 52 - DNA, 20 bp, synthetic construct
  source 1..20: mol_type = other DNA
ggagattcacaccggagtca

SEQ ID NO: 53 - DNA, 19 bp, synthetic construct
  source 1..19: mol_type = other DNA
agcgaacgcacatcaagac

SEQ ID NO: 54 - DNA, 20 bp, synthetic construct
  source 1..20: mol_type = other DNA
ctgtaggcgatctgttgggg

SEQ ID NO: 55 - DNA, 23 bp, synthetic construct
  source 1..23: mol_type = other DNA
tatgagcctcgaatccacatagt

SEQ ID NO: 56 - DNA, 22 bp, synthetic construct
  source 1..22: mol_type = other DNA
cctcgttctgataagcagtcac

SEQ ID NO: 57 - DNA, 21 bp, synthetic construct
  source 1..21: mol_type = other DNA
gacctcaagacccgatacctg

SEQ ID NO: 58 - DNA, 21 bp, synthetic construct
  source 1..21: mol_type = other DNA
tagacgagttccgagtccttc

SEQ ID NO: 59 - DNA, 21 bp, synthetic construct
  source 1..21: mol_type = other DNA
gaagcagggctactacaacca

SEQ ID NO: 60 - DNA, 21 bp, synthetic construct
  source 1..21: mol_type = other DNA
cggcctcattgttatgcaggt

SEQ ID NO: 61 - DNA, 20 bp, synthetic construct
  source 1..20: mol_type = other DNA
tgtgcggtgacaacctcaag

SEQ ID NO: 62 - DNA, 20 bp, synthetic construct
  source 1..20: mol_type = other DNA
acaggagcctgatacgccat

SEQ ID NO: 63 - DNA, 22 bp, synthetic construct
  source 1..22: mol_type = other DNA
gtgacttcaaggactgtgaacg

SEQ ID NO: 64 - DNA, 21 bp, synthetic construct
  source 1..21: mol_type = other DNA
cgggagaactttcgctgacaa

(19) United States
(12) Patent Application Publication
(10) Pub. No.: US 2026/0258369 A1
(43) Pub. Date: Sep. 3, 2026

(54) GENERATION OF BRANCHING URETERIC BUD AND COLLECTING DUCT ORGANOIDS FROM HUMAN PLURIPOTENT STEM CELLS

(71) Applicant: The Brigham and Women's Hospital, Inc., Boston, MA (US)

(72) Inventors: Joseph V. Bonventre, Wayland, MA (US); Kyle W. McCracken, Brighton, MA (US); Min Shi, Newport, KY (US)

(21) Appl. No.: 19/164,112

(22) PCT Filed: Mar. 26, 2024
(86) PCT No.: PCT/US2024/021458

§ 371 Date: Sep. 10, 2025

Related U.S. Application Data
(60) Provisional application No. 63/454,693, filed on Mar. 26, 2023.

(51) Int. Cl. C12N 5/071

(52) U.S. Cl. CPC C12N 5/0684; C12N 2500/38; C12N 2501/115; C12N 2501/119; C12N 2501/13; C12N 2501/15; C12N 2501/155; C12N 2501/16; C12N 2501/727; C12N 2506/45; C12N 2513/00

(57) ABSTRACT

Described herein are methods for providing an artificial branching ureteric bud (UB) organoids and collecting duct (CD) organoids, the organoids themselves and cells therefrom, as well as methods of using the same. These organoids being formed from pluripotent stem cells (PSCs) that are mammalian and can in some instances be human PSCs. Certain uses thereof can be screening test compounds and there effects on the aforementioned organoids.

### CLAIM OF PRIORITY

This application claims the benefit of U.S. Provisional Application Ser. No. 63/454,693, filed on Mar. 26, 2023. The entire contents of the foregoing are incorporated herein by reference.

### FEDERALLY SPONSORED RESEARCH OR DEVELOPMENT

This invention was made with Government support under Grant Nos. TR002155, DK39773, DK00772, and DK072381 awarded by the National Institutes of Health. The Government has certain rights in the invention.

### SEQUENCE LISTING

This application contains a Sequence Listing that has been submitted electronically as an XML file named 29618-0330WO1_SL_ST26.xml. The XML file, created on Mar. 25, 2024, is 57,438 bytes in size. The material in the XML file is hereby incorporated by reference in its entirety.

### TECHNICAL FIELD

Described herein are methods for providing artificial branching ureteric bud (UB) organoids and collecting duct (CD) organoids, the organoids themselves and cells therefrom, as well as methods of using the same.

### BACKGROUND

The directed differentiation of human pluripotent stem cells (hPSCs) into organoids has been enabled by application of developmental knowledge and 3D growth environments^{1,2}. The derivation of kidney tissue poses particular challenges in that the organ exhibits sophisticated architecture and comprises two embryologically distinct progenitor tissues. The ureteric bud (UB) and metanephric mesenchyme arise from anterior and posterior mesodermal populations, respectively, which combine in the posterior region of the embryo to form the kidney. Normal organogenesis hinges on the hallmark branching morphogenesis of the UB, which drives the growth and radial organization of the developing fetal kidney. The resulting epithelium forms the urinary collecting system, including the ureter, renal pelvis, renal calyces and the collecting ducts (CDs) of the kidney, which play essential roles in water, electrolyte, and acid-base homeostasis. The metanephric mesenchyme, on the other hand, is successively induced by UB-derived signals into epithelialized nephrons³. Early successes in hPSC-based kidney differentiation focused on metanephric-like tissues, generating kidney organoids that contain multiple nephron components including glomeruli, proximal tubules, and distal tubules^{4,5}. However, these organoids lacked fundamental features of renal development, such as branching morphogenesis and maintenance of a progenitor niche, most likely because they lacked UB structures and the important RET⁺ tip domains. Given the essential roles of the UB in development and in kidney tissue engineering, the field requires efficient methods for differentiating hPSCs into UB organoids.

### SUMMARY

Described herein are methods for generating kidney organoids from induced mesodermal precursors (also referred to herein as mesendodermal progenitor cells (MPCs), generally obtained from pluripotent stem cells (PSCs)). In contrast to present methods involving an extended 3-6 day period of WNT/β-catenin activation^{4, 5, 53, 54} to induce posterior mesoderm, as shown herein, the MPCs are achieved through rapid induction and subsequent differentiation of the primitive streak state. This permits the efficient generation of pronephric intermediate mesoderm (IM) cells, from which the ND is formed. In this protocol, we exposed hPSCs to activators of WNT/β-catenin, FGF, BMP, and TGFβ signaling pathways for one day to induce TBXT⁻ mesendodermal precursors. These progenitors are then immediately differentiated via activation of retinoic acid (RA) and FGF pathways and inhibition of BMP and TGFβ signaling, which represses alternative fates including lateral plate mesoderm and endoderm, respectively. These steps result in the formation of PAX2⁺/GATA3⁺ pronephric IM cells at nearly 90% efficiency.

To generate three-dimensional spheroids, we aggregate the IM cells at day 3 of differentiation using low-attachment plates. Over the subsequent 3-4 days the spheres organize into two distinct domains that we characterized extensively. The major population maintains expression of PAX2/GATA3 and expresses markers of the ND including ALDH1A3²⁵, and these are the cells that will form the UB organoids. The other cells in the spheroids at day 7 exhibited a transcriptional signature consistent with a stromal progenitor-like phenotype including expression of PDGFRA, although the ultimate potential of this population has not been explored.

The morphogenesis of the ureteric tree is driven by bifurcative branching at the UB tips, which in vivo is directed by the neighboring cap mesenchyme cells from the MM. Key paracrine factors that promote branching include GDNF and FGF signals^{28, 29, 55}. However other factors are also necessary as was originally shown in culture experiments using isolated rodent UBs, which required conditioned medium from an immortalized metanephric cell line (as well as exogenous GDNF and FGF1) to induce branching behavior²⁸. While other reports have now identified combinations of growth factors and inhibitors that support growth and branching of the isolated UB^{8, 27}, we applied modified, and in many steps simplified, approaches to optimize, and make more robust, the growth of hPSC-derived UB organoids. In our protocol, we embed the ND spheroids at day 6 or 7 into a three-dimensional extracellular matrix and overlay media containing multiple factors. The structures then adopt a UB phenotype and undergo several rounds of branching over one week of culture. During this period the organoids adopt a tip-stalk organization comparable to the fetal UB, with localization of the receptor RET in specialized tip domains⁸.

Provided herein are in vitro methods that use specific factors to promote development of ureteric bud (UB) organoids and collecting duct (CD) organoids, preferably with serum-free defined media, examples of which are provided herein.

Thus, provided herein are methods for providing an artificial branching ureteric bud (UB) organoid from a population of mammalian pluripotent stem cells (PSC). The methods comprise (i) culturing the PSC to induce formation of a population of cells comprising at least 90%, 91%, 92%, 93%, or 94% TBXT-positive mesendodermal progenitor cells (MPCs); (ii) culturing the population of MPCs in a serum-free growth medium in the presence of retinoic acid (RA) or an analog thereof, fibroblast growth factor 2 (FGF2), an inhibitor of BMP, and an inhibitor of TGFβ signaling for about 48 hours to induce formation of a population of cells comprising at least 85% or 86% PAX2-positive/GATA3-positive/LHX1-positive intermediate mesoderm (IM) progenitor cells (IMPCs) for about 28-32 hours; (iii) promoting aggregation of the IMPCs into spheroids, preferably spheroids having a diameter between 50-200 μm; (iv) culturing the IMPC spheroids in the presence of a serum-free growth medium containing only RA, or an analog thereof, and FGF9 for about two days to form spheroids comprising GATA3-positive/PAX8-positive cells; (v) culturing the nephric duct spheroids in a serum-free growth medium in the presence of RA or an analog thereof, and glial cell line-derived neurotrophic factor (GDNF) for about two days to form a population of nephric duct spheroids comprising GATA3-positive/PAX2 positive/RET-positive cells; (vi) embedding the nephric duct spheroids in a natural or synthetic hydrogel scaffold, preferably comprising a natural extracellular matrix (ECM), and (vii) culturing in the presence of media comprising FGF10, GDNF, a Wnt agonist, a BMP inhibitor, a TGF-β type I inhibitor, RA, and a MEK inhibitor, and optionally a ROCK inhibitor, for 4-10 days; thereby providing an artificial UB organoid.

In some embodiments, the PSC is a human PSC (hPSC).

In some embodiments, step (i) comprises culturing the PSC in the presence of a WNT agonist, preferably a GSK3β inhibitor, optionally CHIR99021 or BIO; FGF2; BMP4; and TGFβ, preferably Activin A, e.g., WNT agonist CHIR99021 (2-10 μM), FGF2 (10-200 ng/ml), BMP4 (10-200 ng/ml), and TGFβ (e.g., Activin A (10-200 ng/ml)).

In some embodiments, step (ii) comprises culturing the population of MPCs in the presence of retinoic acid (RA) or TTNBP; FGF2; an inhibitor of BMP selected from LDN193189, DMH-1, or dorsomorphin; and an inhibitor of TGFβ signalling selected from A8301 or SB-431542.

In some embodiments, promoting aggregation of the IMPCs into spheroids in step (iii) comprises plating the cells at low density on a low attachment substrate or on a patterned microwell plate.

In some embodiments, step (iv) comprises culturing the IMPC spheroids in the presence of a serum-free growth medium containing only RA and human FGF9.

In some embodiments, step (v) comprises culturing the nephric duct spheroids in a serum-free growth medium in the presence of RA and human GDNF.

In some embodiments, the natural or synthetic hydrogel scaffold in step (vi) comprises natural extracellular matrix (ECM).

In some embodiments, step (vii) comprises culturing in the presence of media comprising human FGF10; human GDNF; a Wnt agonist selected from CHIR99021 or BIO; an inhibitor of BMP selected from LDN193189, DMH-1, or dorsomorphin; an inhibitor of TGFβ signalling selected from A8301 or SB-431542; RA; and MEK inhibitor U0126, and optionally ROCK inhibitor Y-27632.

In some embodiments, the methods further comprise: (viii) incubating the artificial UB organoid in media comprising arginine vasopressin (AVP) and aldosterone (Aldo) for about 3-4 days, to induce formation of collecting duct (CD) organoids comprising AQP2-positive principal cells (PCs).

In some embodiments, the methods further comprise: (ix) inducing FOXI1 expression in the CD organoids for about four days, to induce differentiation of ATP6V1B1-positive intercalated cells (ICs).

In some embodiments, the methods do not comprise cell sorting or purification to obtain a UB organoid or CD organoid.

In some embodiments, the methods further comprise dissociating the cells of the UB or CD organoid.

Additionally, provided herein are artificial UB and CD organoids obtained by a method described herein.

Further, provided herein are isolated cells obtained from an artificial UB or CD organoid obtained by a method described herein, optionally a principal cell (PC) or an intercalated cell (IC) obtained from a CD organoid, or a PAX2-positive/GATA3-positive/RET-positive cell from a UB organoid.

Also provided herein are methods of screening a test compound. The methods can include performing a method described herein for generation of UB or CD organoids in the presence and absence of a test compound, and determining an effect of the test compound on development of the UB or CD organoids. Alternative, the methods can include maintaining a UB or CD organoid obtained by a method described herein in the presence and absence of a test compound, and detecting an effect of the test compound on a parameter of the organoid, e.g., on function or expression of a selected marker.

Also provided herein are methods to generate ureteric bud 3D organoids and functional collecting duct cells from human progenitor stem cells (hPSCs) comprised of the following (details of which are disclosed herein): (i) monolayer induction of hPSCs into PAX2+/GATA3+ cells having a pronephric intermediate mesoderm fate; (ii) aggregation of said cells into 3D spheroids; (iii) growth of ureteric bud organoids from said spheroids when embedded in an extracellular matrix-like culture environment; and, optionally, (iv) permissive differentiation of said organoids to form collecting duct principal cells. In some embodiments, said PAX2+/GATA3+ cells in step (i) are induced from hPSCs by presenting hPSCs to media containing activators of WNT/β-catenin, FGF, BMP, and TGFβ signaling pathways followed by selective differentiation with activation of retinoic acid and FGF pathways and concomitant inhibition of BMP/TGFβ signaling. In some embodiments, the PAX2+/GATA3+ cells are aggregated into 3D spheroids as in step (ii) above by plating said PAX2+/GATA3+ cells at low density on a low attachment substrate. In some embodiments, ureteric bud organoids are generated from 3D spheroids as in step (iii) above by plating spheroids in dishes containing a 3D matrix composition and media supplemented with some combination of FGF10, GDNF, CHIR, LDN193189, A83-01, Retinoic Acid and U0126 (see below). In some embodiments, the 3d matrix composition is Matrigel. In some embodiments, the organoids are dissociated into differentiated human collecting ductal cells that are unique in their ability to generate a high resistance transepithelial resistance and electogenic transport which results in transepithelial voltage difference as in step (iv) above by removal of said supplements from the medium and/or transfected with an inducible transcription factor (e.g., FoxI1).

Also provided herein are cell lines as produced by a method described herein, e.g., as described in the preceding paragraph.

Further provided herein are methods using the cells or cell line of any of the preceding claims, to screen for therapeutic drugs or druggable targets in disease states that involve the kidney collecting duct such as polycystic kidney disease. The collecting duct cells can be used to generate three-dimensional single layer epithelial structures (tubuloids) from the cells generated as described herein. Said cells and tubuloids can be used to create a model of cytogenesis thereby allowing the interrogation of mechanisms underlying cyst formation and discovery of drugs that inhibit or reduce said cyst formation.

Additionally, provided herein is a method, using the cells, cell line or tubuloids as described herein, to screen for therapeutics for hypertension and abnormalities of sodium, acid base regulation and potassium handling by the collecting duct. Cells and derived tubuloids generated as described herein can be exposed to a candidate drug and the effects of candidate drug on electrogenic properties of said cells assayed.

Also provided herein are methods to screen for therapeutics for interstitial fibrosis and chronic kidney disease comprised of interrogating cells, generated as described herein, for physiological responses associated with fibrotic pathology and drug screening.

Additionally, provided herein is an in vitro model system of congenital abnormalities of the kidney and urinary tract comprised of the cells, generated as described herein, that can be or have been modified with gene editing technologies, e.g. CRISPR, zinc finger nucleases, or TALENS, to assess genetic influences and mutation on kidney development.

Further, provided herein is a screening and drug discovery platform for drug toxicity studies comprised of measuring the effects of drugs or other agents on the responses of cells, generated as described herein, and systems that can employ said cells (e.g., microfluidics).

Further, provided herein is a screening platform for drug discovery directed to kidney diabetes insipidus, tubulointerstitial fibrosis, or chronic kidney disease comprised of measuring the ability of effective agents or drugs to counteract the effects of lithium toxicity in cells generated as described herein.

Unless otherwise defined, all technical and scientific terms used herein have the same meaning as commonly understood by one of ordinary skill in the art to which this invention belongs. As used herein, the term “about” means plus or minus 10%, unless otherwise specified. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### DESCRIPTION OF DRAWINGS

FIGS. 1A-I. Directed differentiation of hPSCs into pronephric IM and nephric duct spheres. a, Schematized diagram of stepwise differentiation strategy for generation of ureteric bud and collecting duct organoids. b, Immunofluorescent (IF) staining of monolayer cultures at day 1 revealed efficient induction of TBXT-expressing mesendodermal progenitor cells. c, Gene expression analyses by qPCR revealed dynamic down- and up-regulation of pluripotency (NANOG) and primitive streak (TBXT) markers, respectively, after one day of induction. Following two subsequent days of exposure to IM-inducing factors, TBXT was repressed and there was robust activation of PAX2, PAX8, and GATA3. n=3 independent biological replicates per timepoint. d, IF staining of day 3 monolayer cultures demonstrated a high proportion of cells with co-expression of pronephric IM genes PAX2, PAX8, GATA3, LHX1, and HOXB7. e, Quantification of staining for GATA3 and PAX2 represented on histogram revealed a high efficiency of differentiation into pronephric IM fate. n=7 quantified fields from 3 independent biological replicates. f, Following aggregation of IM cells at day 3, the resulting structures underwent stereotypic morphogenetic events characterized in the time course of stereomicrographs and fluorescent imaging using GATA3-mScarlet reporter. By day 7, the GATA3⁺ cells arranged into a dense sphere that excluded a minority population of negative cells. g, IF staining of sections of nephric duct spheres at days 4 and 7 demonstrated maintenance of expression of PAX2, PAX8, and GATA3. h, UMAP representing scRNA-seq of cells isolated from spheres at day 7, which demonstrated the presence of both nephric duct (clusters 2 and 3) and stromal (clusters 0 and 1; PDGFRA⁺) lineages. i, The majority of cells at day 7 expressed the nephric duct leader fate marker ALDH1A3 with low levels of the mature epithelial marker CDH1. Scale bars 100 μm (b and d), 50 μm (f, g and i). Column and error bars represent mean and standard deviation, respectively.

FIGS. 2A-H. Three-dimensional development of branching UB organoids. a, Between days 7-14, the nephric duct spheres underwent extensive remodeling and growth into UB organoids, which grew in a pattern of elongating stalks and branching tips similar to the fetal UB. b, Bifurcative branching was frequently observed over this time period. c, Molecular analysis at day 14 reiterated a complex branched architecture and the entire epithelium exhibited expression of GATA3, GRHL2, SOX9, and CDH1. The progenitor marker and GDNF co-receptor RET was exclusively expressed in in the distal tip domains. d, Wholemount IF staining at days 9, 11, and 14 revealed uniform expression of PAX2 with progressive segregation of the tip (RET) and stalk (KRT8) domains. e, In the absence of exogenous GDNF, the nephric duct spheres largely failed to grow and branch. Conversely, removal of the MEK inhibitor U0126 from the culture medium led to a more disorganized branched plexus of fine epithelial cords with terminal filopodia rather than rounded UB-like tips. f, To functionally assess the competence of UB organoid cells, we mixed them with dissociated murine kidneys isolated at embryonic day (E) 12.5, pelleted the cells into chimeric aggregates, and cultured on filter disks for 72 hours. g, IF staining showed the incorporation of induced UB cells (indicated by a human-specific antibody) into the Cdh1-expressing UB tip domains but not in the Six2⁻Wt1⁺ nephron progenitor compartment. h, Histogram depicting the frequency at which we observed a human cell or cluster of cells in the examined progenitor niches. Scale bars, 200 μm (a and c-e), 100 μm (b) and 20 μm (g).

FIGS. 3A-J. Differentiation of collecting duct epithelia from UB organoids. a, Illustration demonstrating that the collecting duct epithelia comprise a mixture of principal cells, A-type, and B-type intercalated cells. b, Expression of markers ELF5, AQP2, SCNN1B, and SCNN1G increased over time, consistent with spontaneous formation and maturation of principal cells. When organoids were transitioned to a basal medium containing only AVP (10 nM) and Aldosterone (10 nM) from days 14-18, there was a marked increase in the expression of each of these genes. Dotted line represents expression observed in a sample of human kidney cortex. n=3 independent biological replicates per time point/condition. c, When the UB organoids were transitioned to the basal medium (with AVP/Aldo), the progenitor marker RET was downregulated in the epithelial tip domains, while AQP2 expression became more widespread and expressed at strikingly higher levels by IF. d, In numerous instances, AQP2 was localized in the epithelia nearer to the luminal or apical membrane, albeit it was not strictly apically localized. e, UMAP of scRNA-seq analysis of day 18 organoids that were cultured for four days in basal media with AVP/Aldo. f, Violin plots demonstrate expression of principal cell markers in clusters 0-4, which appear to be related given close association on UMAP, but not in cluster 5. g-h, Accordingly, integration and reference mapping to adult human kidney dataset predicted an ‘inner medullary collecting duct’ in 86.9% of cells in the dataset. While clusters 0-2 were nearly uniformly mapped to a collecting duct fate, clusters 3 and 4 contained a small proportion of cells that mapped to alternative tubular fates, namely the descending thin limb and distal convoluted tubule. i-j, Lineage trajectory analysis in Monocle predicted clusters 0 and 1 to be most differentiated cell types, while clusters 3 and 4 represented the earliest developmental stages in pseudotime. Scale bars, 100 μm (c) and 20 μm (d). Column and error bars represent mean and standard deviation, respectively.

FIGS. 4A-I. hPSC-derived model for interrogating ENaC-mediated sodium transport. a, Expression of the ENaC subunits SCNN1B and SCNN1G was abundant in scRNA-seq data, and we detected SCNN1B at the protein level in day 18 organoids. b, To explore functional capabilities, organoids at day 18 were dissociated and plated onto two-dimensional transwell filters for electrophysiological interrogation. This led to confluent epithelia that maintained expression of collecting duct markers PAX2 and CDH1. c-d, As the cells became confluent, there was at first a steep increase and then plateau in the transepithelial resistance, which was then followed by the emergence of a transepithelial voltage. The voltage was entirely suppressible within three minutes of addition of the ENaC antagonist amiloride (10 μM), which also raised the resistance across the epithelium. e, The resulting current generated was calculated using Ohm's Law, and it was completely ablated immediately following addition of amiloride, confirming the conductance was ENaC-dependent. * p=5.3×10⁻⁹, **p=5.7×10⁻⁸, ***p=4.5×10⁻⁶; two-tailed student's t-test comparing results before and after amiloride; n=6 biological replicates, data representative of 3 independent experiments, f, Under closed-circuit conditions with voltage clamping in Ussing chamber, the current produced by the epithelium was similarly sensitive to amiloride, and it was completely abolished upon increasing the concentration to 100 μM. g, Amiloride induced a dose-dependent inhibition of transepithelial current in Ussing chamber, with representative dose-response curves shown from two independent wells. h-i, Addition of aldosterone for 24 hours led to a dose-dependent increase in transepithelial current measured under closed circuit conditions, and up to a maximum of approximately 2.5-fold increase in amiloride-sensitive current. *p<0.005; two-tailed student's t-test compared to no aldosterone; n=4 independent biological replicates. Scale bars, 100 μm (a) and 20 μm (b). Column and error bars represent mean and standard deviation, respectively.

FIGS. 5A-M. FOXI1 expression is sufficient for intercalated cell fate determination. a, hESCs were transduced with lentivirus encoding a doxycycline (dox)-inducible hFOXI1 construct, and the cells were differentiated into UB organoids. b, In the undifferentiated state, FOXI1 expression was not observed in the absence of dox, but it was widely induced after exposure to dox for 48 hours. c, While control organoids exhibited no expression of FOXI1 or IC markers, the short exposure to dox induced formation of FOXI1- and ATP6V1B1-expressing ICs in a salt-and-pepper distribution. AQP2 expression was maintained in some cells but was mutually exclusive with the induced ICs. d, In the dox-induced organoids at day 18, the epithelium comprised an assortment of AQP2-expressing principal cells and ATP6V1B1-positive ICs. e, In the dox-induced organoids, only B-ICs were observed that expressed SLC26A4, while there were no SLC4A1-expressing A-ICs. f, In the 2D cell culture model, doxycycline-treated cells exhibited significant reduction in transepithelial resistance. *p<0.0005; two-tailed student's t-test. g-h, While control cells had a negative transepithelial voltage and current that was suppressed by amiloride, the doxycycline-treated cells had a positive baseline potential and current that was augmented by amiloride but completely suppressed by the V-ATPase inhibitor bafilomycin (10 nM). *p=2×10⁻⁶, **p=1.3×10⁻⁷, two-tailed student's t-test (h). i, Therefore control cells displayed amiloride-sensitive current while doxycycline induced a predominantly bafilomycin-sensitive current. *p=2.5×10⁻⁴, **p=1.0×10⁻⁶, two-tailed student's t-test. In f-i, n=9 independent biological replicates per condition over two separate experiments, j, Experiments in Ussing chamber confirmed that FOXI1 expression resulted in bafilomycin-sensitive rather than amiloride-sensitive short-circuit current. Shown are representative tracings from two independent experiments. Asterisks indicate 1V pulses to assess transepithelial resistance. k, Doxycycline-treated cells induced acidification of the cell culture medium, which (1) was only observed in the apical chamber of the transwells. m, Measured using a pH meter, the FOXI1-induced cells generated a pH gradient of ~0.5 units between the upper and lower chambers of the transwell. N=9 independent biological replicates per condition. *p=1.6×10⁻⁹, **p=0.007, ***p=7.8×10⁻⁹, two-tailed student's t-test. The data shown in k-m have been observed in more than 8 independent experiments. Scale bars, 50 μm (b-c) and 100 μm (d-e). Column and error bars represent mean and standard deviation, respectively.

FIG. 6. Generation of GATA3-mScarlet reporter allele. Schematic representation depicting targeting scheme for CRISPR/Cas9-mediated knock-in of mScarlet into human GATA3 locus in H9 hESC line. The donor vector containing P2A-mScarlet and Hygro resistance cassette with flanking homology arms was co-transfected into cells with plasmid coexpressing Cas9 and gRNA targeting GATA3 stop codon.

FIGS. 7A-F. Efficient induction of pronephric IM cells at day 3. a, Brightfield micrographs demonstrating appearance of undifferentiated hESCs at day 0, as well cultures after 24 and 30 hours exposure to primitive streak-inducing factors. At 24 hours, there was still significant colony-like morphology consistent with incomplete induction of mesendoderm cells, but after six additional hours the colonies were nearly completely dissociated into single, mesenchymal-like cells. b, Quantification of IF staining for TBXT (as shown in FIG. 1b) revealed >95% efficiency after 30 hours exposure. n=6 quantified fields from three independent replicates. c, Timecourse qPCR analysis corresponding to FIG. 1c. The primitive streak marker MIXL1 was maximally expressed at day 1 and was subsequently quickly down-regulated, while IM markers OSR1, HOXB7, HNF1B, and SOX9 were increased by day 3. n=3 independent biological replicates per timepoint. d, Efficient specification and expression of pronephric IM genes at day 3 was dependent on the combinatorial effects of FGF2, RA, TGFβ inhibition (A83-01), and BMP inhibition (LDN193189) during days 1-3. n=3 independent biological replicates per condition. e, In the pronephric IM cultures at days 3 and 7, there was low level of expression of posterior IM markers (WT1, SIX2, EYA1, and HOXA11), which were derived from a differentiation protocol for inducing metanephric progenitor cells. *, p<0.05; **p<0.005; two-tailed Student's t-test individually comparing day 3 and day 7 against the day 9 posterior IM samples; n=4 biological replicates, data representative of 2 independent experiments. Specifically p-values for comparisons using day 3 were 0.005, 0.006, 0.0005, and 0.017 for WT1, SIX2, EYA1, and HOXA11, respectively; at day 7, they were 0.007, 0.007, 0.0002, and 0.015. f, From days 1-3, the TGFβ inhibitor A8301 was required for suppression of definitive endoderm (SOX17) fate, whereas BMP inhibition with LDN193189 inhibited formation of lateral plate mesoderm (FOXF1). *p<0.005; two-tailed Student's t-test; n=3 biological replicates, data representative of 2 independent experiments. Scale bar 200 μm (a). Column and error bars represent mean and standard deviation, respectively.

FIGS. 8A-I. Characterization of nephric duct spheroids. a, Pronephric IM cells aggregated at day 3 efficiently formed numerous compact spheroids that maintained high levels of GATA3 expression over the course of their development. b, When either FGF2 or FGF8 was used in place of FGF9 from days 3-5, the spheres were more loosely organized and exhibited lower expression of GATA3. c, Micrograph of a small region of nephric duct progenitors plated on a patterned microwell, demonstrating high degree of uniformity of size, shape, and structure of the spheroids. d, From days 4-7, the spheres maintained expression of early pronephric transcription factors LHX1 and HOXB7, and they also gradually acquired expression of nephric duct markers RET and EMX2. e, Over these several days of culture, qPCR analysis showed developmental increase in nephric duct genes RET, WNT11, WNT9B, and EMX2. n=3 independent biological replicates per timepoint. f-g, Heatmap and expression plots representing scRNA-seq data from day 7 revealed high expression of nephric duct markers in clusters 2 and 3, with complementary expression of stromal lineage genes in clusters 0 and 1 (in reference to UMAP in FIG. 1f). h, IF staining confirmed a high level of WT1-positivity in nephric duct lineage-negative cells at day 7. i, The differentiation of off-target lineages was not observed in the scRNA-seq dataset. Scale bars, 500 μm (a), 100 μm (b), 400 μm (c), 50 μm (d and h). Column and error bars represent mean and standard deviation, respectively.

FIGS. 9A-F. UB organoid differentiation protocol is efficient when using hiPSCs. a, Using the hiPSC line BJFF.6, a 30 hour exposure during the first stage of differentiation was also required for the primitive streak phenotype. b, AIM transcription factors PAX2, GATA3, LHX1, and HOXB7 were induced with very high efficiency by day 3. c, The hiPSC-derived AIM exhibited efficient formation of nephric duct spheroids in 96-well low attachment plates. The spheroids then underwent similar molecular and morphological development compared to those derived from hESCs as shown in FIG. 1. d, Nephric duct spheroids efficiently grew into branched UB organoids. e-f, hiPSC-derived UB organoids exhibited tip-stalk patterning and spontaneously formed differentiated AQP2-positive principal cells. Scale bars, 50 μm (a-d) and 100 μm (e-f).

FIGS. 10A-G. Branching morphogenesis in UB organoids. a, After embedding in 3-D matrix at day 7, the UB organoids exhibited several rounds of iterative branching during the first week of culture. The stereomicrograph of day 11 UB organoid demonstrates multiple terminal branching events, with each generation uniquely colored: 1=green, 2=red, 3=blue, 4-orange. b, A characteristic terminal bifurcation was demonstrated in the time course of micrographs. c, At later stages, such as day 18, branching was slowed and instead the distal UB tips formed enlarged knobs that more rarely underwent further cleavage events. d-e, Wholemount IF staining at days 9, 11, and 14 as shown in FIG. 2c with separation of channels. f, Expression of the transcription factor HNF1B increased between days 9 and 14. g, From day 7, the UB organoids showed a gradual decline in the tip marker WNT11 and a corresponding increase in the medullary stalk marker WNT7B. WNT9B, which is more broadly expressed in the stalk components, was maintained at relatively stable levels. n=3 independent biological replicates per timepoint. Scale bars, 50 μm (a-c) and 200 μm (d-f). Column and error bars represent mean and standard deviation, respectively.

FIGS. 11A-D. UB progenitor cells participate in niche interactions in chimeric explants but not with hPSC-derived metanephric cells. a, Cells from induced UB organoids and metanephric kidney organoids were dissociated at day 7, mixed and reaggregated, and cultured as spheres in suspension. Within two days (at day 9) the GATA3/KRT8-expressing UB cells had formed an epithelial network in the inner portion of the organoid. Metanephric cells (PAX2 only) were differentiating around the periphery of the structure in close association with the UB cells. However, we did not observe formation of capping mesenchyme structures or branching within the UB epithelium. b, Brightfield image with visualization of the UB epithelium using GATA3-mScarlet reporter confirmed the absence of significant branching morphogenesis. c-d, In chimeric explants with mouse fetal kidneys, the human induced UB cells (indicated by human nuclear antigen detection) incorporated into the UB tip (Cdh1-positive) at a high frequency, but never into the surrounding metanephric progenitors (Wt1 and Six2). Shown are six representative examples of progenitor niches that contained human cells. Scale bars, 200 μm (a and b) and 20 μm (c and d).

FIGS. 12A-C. Schematized depiction of protocol for directed differentiation of UB organoids. a, Derivation of pronephric IM progenitor cells in three days in monolayer culture format. b, Formation of ND spheroids in either 96-well plate or AggreWell-400 microwell plate from day 3 to 7. c, Embedding of spheroids in Matrigel for 3D culture and growth from day 7 to 18.

FIGS. 13A-D. Morphologic appearance of starting cultures. a, hPSCs are routinely passaged as colonies or clumps approximately 50-200 μm in size. b, Healthy hPSC colonies exhibit flat appearance with tightly packed small cells with high nuclear-to-cytoplasmic ratio. c, In contrast, differentiated cells appear larger with a looser organization, and we typically observe them within the edges of the colonies. Colonies with excessive differentiation as shown should be manually removed prior to passaging or plating for differentiation. d, Representative micrographs showing the desired density of cultures on day 0, on which with the cells appearing as cords or small colonies at a fairly low density (20-30% confluence). Also shown are cultures that are either too sparse or too dense to result in an efficient differentiation. Scale bars, 100 μm (a, d), 200 μm (b-c).

FIG. 14. Duration of mesendoderm induction step is critical for efficient formation of pronephric IM cells. On day 1, cultures should be monitored carefully for the morphologic changes depicted. The IM Medium should be added only after much of the colony morphology (outlined in red dashed line in the top row) has been disrupted and the cells largely exhibit a mesenchymal phenotype. If done too early (top row) or too late (bottom row), the efficiency of PAX2/GATA3 induction at day 3 is significantly impaired. Scale bar, 200 μm.

FIGS. 15A-D. Formation of 3D nephric duct spheroids. a, In the 96-well plate format, the initial small aggregates coalesce into larger spheroids by day 5. On day 7, the spheroids are segregated into two domains representing the ND progenitors and a stromal compartment. b, Using a patterned microwell AggreWell-400 plate, the spheroids adopt uniform size and morphology, and the same lineage segregation is observed by day 7. c, The size of ND spheroids can be manipulated in patterned AggreWell-400 plates by altering the plating cell density, and the morphologic changes and segregation are unaffected by starting spheroid size. d, A representative ND spheroid on day 7 in microwell plate with the ND compartment comprising cells that are nearly uniformly positive for PAX2 and GATA3. Scale bars, 200 μm (a-c), 100 μm (d).

FIGS. 16A-F. Morphogenesis and differentiation of UB organoids in 3D culture. a, Brightfield micrographs demonstrating demonstrate the growth pattern of organoids over 7 days of culture after embedding in Matrigel. Also note the scattered loose cells in the left lower portion at days 9-14, which show characteristic dispersion of the spheroid's stromal cells once placed in Matrigel culture. b, Low magnification image of the majority of the Matrigel dome shows typical density of organoids as well as some of the undifferentiated stromal cells (bottom). Note that some organoids appear out of focus since they occupy different planes within the 3D Matrigel. c, UB organoids on day 14 exhibit diffuse expression of PAX2, GATA3, LHX1, SOX9, and CDH1, with localization of RET expression specifically in the distal tip progenitor cells, as shown in both flat preparations of wholemount-stained organoids and immunostained sections of organoids. d, On day 18 after four days of culture in CD Medium, the stalks and branches of the organoid appear more thickened. e-f, The formation of AQP2-positive principal cells is observed throughout the organoids by day 18. Scale bars, 200 μm (a-e), 100 μm (f).

FIG. 17. Optimization of UB Medium for branching in iPSC72-3-derived organoids. When deriving UB organoids from a new cell type, it is useful to perform optimization of the concentrations of U0126 and Y-27632 in the UB Medium. For the iPSC72-3 cell line shown here at day 10, the addition of Y-27632 and omission of U0126 produced the best morphological result at day 10. Scale bar, 200 μm.

### DETAILED DESCRIPTION

The differentiation of UB organoids was initially demonstrated using mouse embryonic stem cells (mESCs); when these organoids were combined with induced metanephric progenitors, the resulting tissues formed branching structures with a high degree of similarity to the developing mouse kidney⁶. Similar results have not been reproduced using human cells. Recent protocols for differentiation of hPSCs to UB tissue have several technical and functional limitations. The early differentiation steps are inefficient, requiring the use of either cell sorting⁶⁻⁸ or mechanical dissection⁹ to enrich for appropriate progenitor cell populations. While UB progenitor cells could be expanded in 3D tissues, they did not reproduce either the morphological⁸⁻¹⁰ or temporal characteristics^{6,7} of the iterative UB branching observed in vivo, often rather forming a folded epithelial structure⁸⁻¹⁰. One approach achieved a stalk morphology with a terminal branching event, but the growth and branching occurred over several weeks of 3D culture^{6, 7}, compared with the normal UB that branches multiple times over several days. Moreover, to our knowledge robust in vivo-like functional quantitative characterization has not been described for hPSC-derived UB lineage cells, and only rarely for any hPSC-derived renal cell type. No hPSC-derived kidney cell types have been convincingly shown to recapitulate authentic regulated kidney physiologic functions. In the CD epithelium, the two major functional cell types are principal cells (PCs) and intercalated cells (ICs). The former regulate sodium, potassium, and water homeostasis, and the latter maintain acid-base equilibrium. Although immortalized cell lines derived from the mouse kidney displayed PC-like behaviors¹¹, such as sodium reabsorption, similarly immortalized primary human cell lines do not¹². Primary ICs from rodent models do not maintain a stable phenotype in cell culture¹³ so there has been no accessible model for in vitro IC function.

Here we present robust methods for the stepwise derivation of three-dimensional UB organoids and CD tissue from PSCs, e.g., mammalian PSCs, preferably human PSCs (hPSCs), or PSCs from non-human primates (NHPs) or non-human veterinary subjects such as cats, dogs, and horses. The present methods have been optimized to ensure at least 90% efficiency at the mesendodermal stage and at least 85% at the pronephric intermediate mesoderm (IM) stage. To obtain a population of cells comprising at least 90%, 91%, 92%, 93%, or 94% TBXT-positive mesendodermal progenitor cells (MPCs), the present methods can include culturing the PSC in the presence of a WNT agonist (2-10 μM), FGF2 (10-200 ng/ml), BMP4 (10-200 ng/ml), and TGFβ (e.g., Activin A (10-200 ng/ml)). The Wnt agonist is preferably a GSK3β inhibitor, optionally CHIR99021 or (2′Z,3′E)-6-Bromoindirubin-3′-oxime (BIO). Other GSK3β inhibitors that can be used include, but are not limited to, Purvalanol A, olomoucine, alsterpaullone, kenpaullone, benzyl-2-methyl-1,2,4-thiadiazolidine-3,5-dione (TDZD-8), 2-thio(3-iodobenzyl)-5-(1-pyridyl)-[1,3,4]-oxadiazole (GSK3 inhibitor II), 2,4-dibenzyl-5-oxothiadiazolidine-3-thione (OTDZT), α-4-Dibromoacetophenone (i.e., Tau Protein Kinase I (TPK I) Inhibitor), 2-Chloro-1-(4,5-dibromothiophen-2-yl)-ethanone, N-(4-Methoxybenzyl)-N′-(5-nitro-1,3-thiazol-2-yl) urea (AR-A014418), and indirubins (e.g., indirubin-5-sulfonamide; indirubin-5-sulfonic acid (2-hydroxyethyl)-amide indirubin-3′-monoxime; 5-iodo-indirubin-3′-monoxime; 5-fluoroindirubin; 5,5′-dibromoindirubin; 5-nitroindirubin; 5-chloroindirubin; 5-methylindirubin, 5-bromoindirubin), 4-Benzyl-2-methyl-1,2,4-thiadiazolidine-3,5-dione (TDZD-8), 2-thio(3-iodobenzyl)-5-(1-pyridyl)-[1,3,4]-oxadiazole (GSK3 inhibitor II), 2,4-Dibenzyl-5-oxothiadiazolidine-3-thione (OTDZT), (2′Z,3′E)-6-Bromoindirubin-3′-oxime (BIO), α-4-Dibromoacetophenone (i.e., Tau Protein Kinase I (TPK I) Inhibitor), 2-Chloro-1-(4,5-dibromo-thiophen-2-yl)-ethanone, (vi) N-(4-Methoxybenzyl)-N′-(5-nitro-1,3-thiazol-2-yl) urea (AR-A014418), and H-KEAPPAPPQSpP-NH2 (L803) or its cell-permeable derivative Myr-N-GKEAPPAPPQSpP-NH2 (L803-mts). Other GSK3β inhibitors are disclosed in U.S. Pat. Nos. 6,417,185; 6,489,344; and 6,608,063. Wnt proteins, e.g., Wnt3a, can also be used. Where a protein is used, preferably the sequence of the protein is from the same species as the PSC; for example, where human PSC are used, human FGF2, BMP4, and TGFβ (e.g., Activin A) are preferred.

In the next stage, the population of MPCs is incubated in the presence of retinoic acid (RA; 100-1000 nM) (or an analog thereof, e.g., (E)-4-[2-(5,5,8,8-tetramethyl-5,6,7,8-tetrahydro-2-naphthalenyl)-1-propenyl]benzoic acid (TTNBP)), fibroblast growth factor 2 (FGF2; 10-200 ng/ml), an inhibitor of BMP (e.g., LDN193189 [100-1000 nM], DMH-1, or dorsomorphin), and an inhibitor of TGFβ signaling (e.g., A8301 [100-1000 nM] or SB 431542) for about 48 hours to induce formation of a population of cells comprising at least 85-90% PAX2-positive/GATA3-positive/LHX1-positive intermediate mesoderm (IM) progenitor cells (IMPCs) for about 25-32 hours.

These progenitor cells then aggregate and form nephric duct spheroids. Aggregation of the cells into spheroids can be promoted by culture on low-attachment plates (e.g., plates with a coating that repels cells, e.g., a hydrophilic neutrally charged coating, e.g., covalent hydrogel coating; a number of suitable plates are commercially available, e.g., from Corning or Cellstar) or patterned microwell plates (e.g., patterned microwell plates comprising a high-density array of microwells, a number of which are commercially available including EZSPHERE, AGGREWELL, ELPLASIA plate, SPHEROFILM, and SPHERICALPLATE 5D). The spheroids can then be developed into branching UB organoids, e.g., by embedding in a natural or synthetic hydrogel scaffold, e.g., comprising natural extracellular matrix (ECM), e.g., MATRIGEL (Corning, Corning, NY), GELTREX LDEV-Free Reduced Growth Factor Basement Membrane Matrix (GIBCO/ThermoFisher), or CULTREX Basement Membrane Extract (BME) (Trevigen); natural scaffolds comprising collagen (e.g., and collagen type IV), fibrin, bone sialoprotein, vitronectin (e.g., VITRONECTIN XF™ (STEMCELL Technologies), or laminin; or combinations thereof. A number of suitable scaffolds are known in the art. See, e.g., Cruz-Acuña and García, Matrix Biol. 2017 January; 57-58 ( ):324-333; Murrow et al., Development. 2017; 144:998-1007; Murphy et al., Nat Mater. 2014; 13:547-557; Nguyen et al., Nat Biomed Eng. 2017; 1:0096; and Aisenbrey and Murphy, Nature Reviews Materials 5:539-551 (2020), and references cited therein.

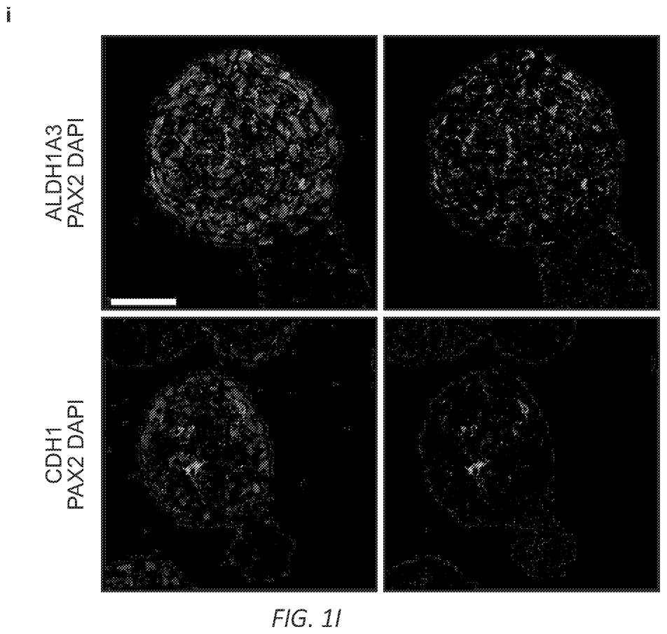

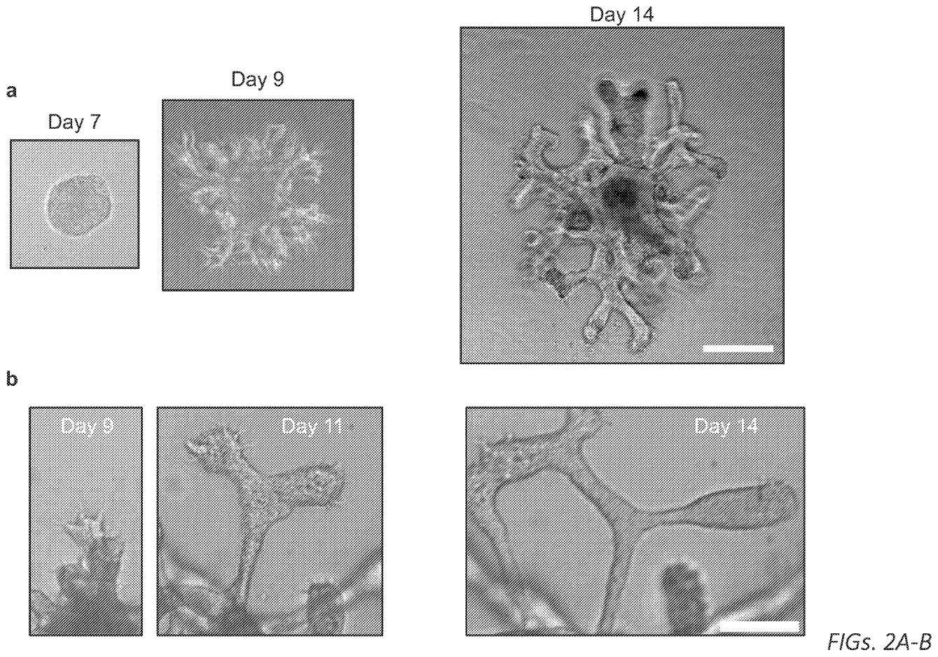

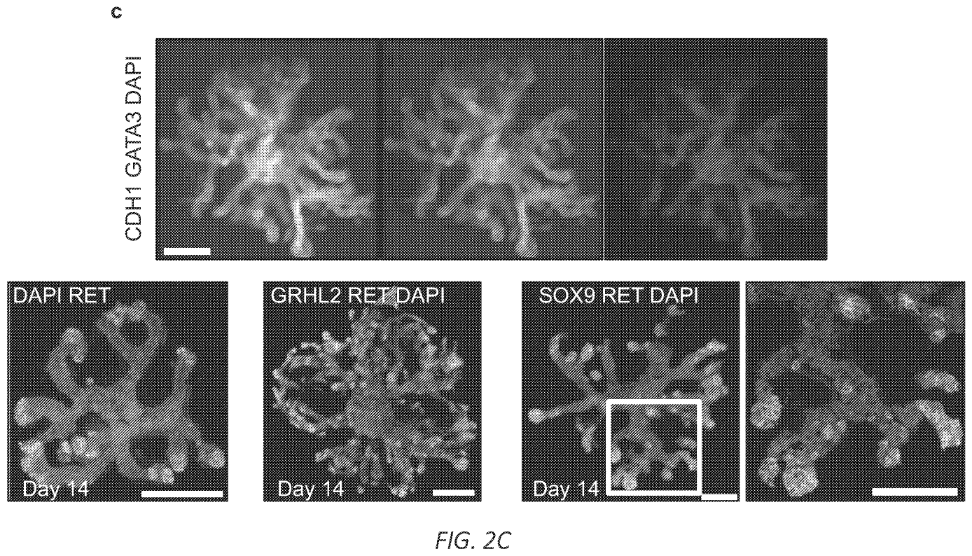

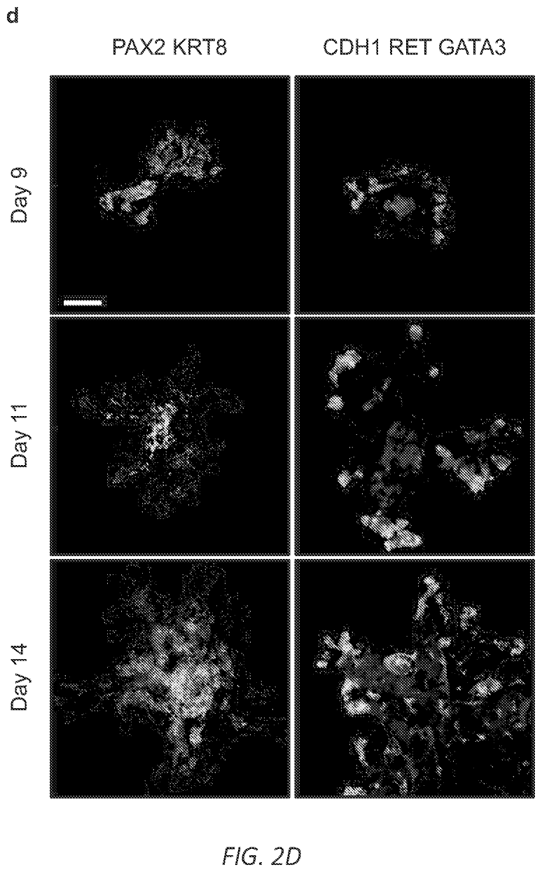

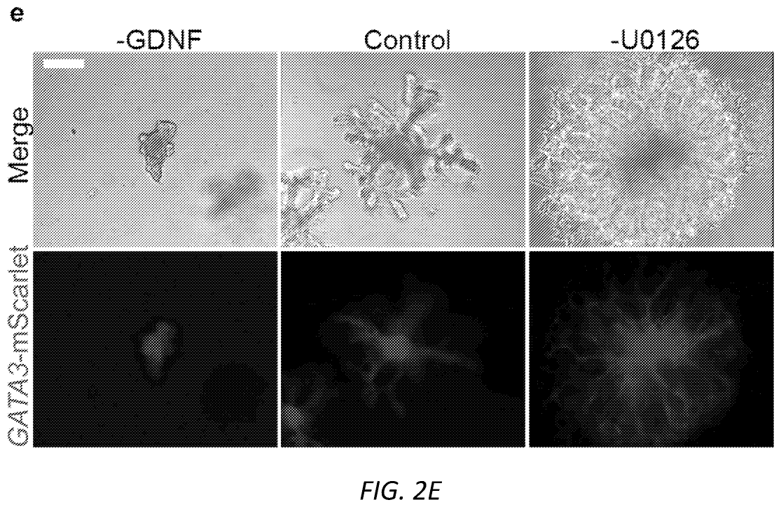

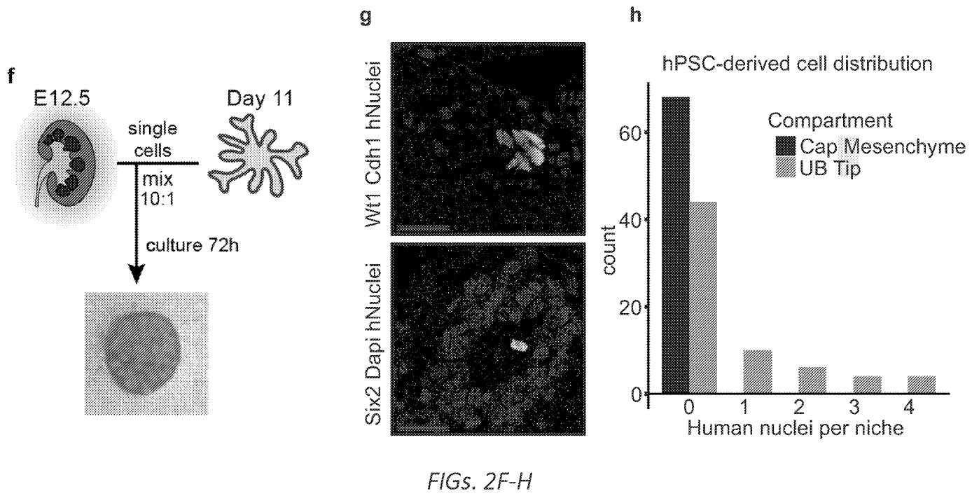

Once embedded in the scaffold, the cells are then maintained in the presence of media comprising FGF10, GDNF, a Wnt agonist, a BMP inhibitor, a TGF-β type I inhibitor, RA, and optionally a MEK inhibitor and/or ROCK inhibitor for about seven days. For example, the media can comprise human FGF10 (10-200 ng/ml); human GDNF (10-500 ng/ml); a Wnt agonist selected from CHIR99021 (1-10 uM) or BIO; an inhibitor of BMP selected from LDN193189 (100-1000 nM), DMH-1, or dorsomorphin; an inhibitor of TGFβ signaling selected from A8301 (100-1000 nM) or SB-431542; RA (100-1000 nM); and optionally a MEK inhibitor U0126 (1-10 μM) and/or ROCK inhibitor Y-27632 (2-10 μM). During this time, the spheroids exhibited a rapidly growing and iterative branching pattern (FIG. 2a). Each of the original ND spheroids generated multiple buds that sprouted in all directions to form elongating tubules, and the epithelia then underwent branching by several rounds of terminal bifurcation. When the cultures were maintained for a week (for a total of 14 days from the induction of formation of mesendodermal progenitor cells (MPCs) from the PSCs), structures formed that included an array of branched epithelial stalks with rounded tips, resembling the ampullae of the fetal UB (FIG. 2c). Beyond day 14, the rate of branching and overall growth in the UB organoids slowed and the bud tips at latter stages sometimes formed enlarged knob-like swellings.

These UB organoids can be further treated to induce formation of collecting duct (CD) organoids comprising AQP2-positive principal cells (PCs), e.g., by incubating the artificial UB organoid in media comprising arginine vasopressin (AVP; 1-100 nM) and aldosterone (Aldo; 1-100 nM) for about 3-4 days; inducing FOXI1 expression in the CD organoids for about four days can further be used to induce differentiation of ATP6V1B1-positive intercalated cells (ICs).

We demonstrated that using the present methods, the generated tissues resembled their respective in vivo analogs at each stage of ureteric development via molecular analysis, and they exhibit authentic morphological behavior and responses to developmental stimuli. Their morphogenetic program is similar to the pattern observed in isolated UBs grown ex vivo¹⁴, with three-dimensional branching and organized tip-stalk polarity, and the cells can integrate into the nephrogenic niche in chimeric fetal kidney explants. Moreover, the UB organoids efficiently differentiated into CD cell types as characterized by scRNA-seq. From these organoids, we derived a 2D PC line that forms a high-resistance epithelium capable of robust sodium transport and a physiologic response to hormone signaling, a functional status not previously achieved in hPSC-derived kidney cells or in cultured primary human CD cells. We also induced the IC fate via expression of FOXI1 to facilitate the study of IC electrophysiology and proton transport. Collectively, these methods enable the modeling of a diverse spectrum of development, physiology, and pathophysiology of the human UB and CD.

We have delineated a systematic directed differentiation strategy for the de novo generation of UB and CD epithelia from hPSCs at unprecedented efficiency and without cell sorting or purification (Table 1).

TABLE 1
Comparison of methods and outcome for UB organoid derivation methods.

| | | | Differentiation | | UB Properties | | | | | | |
|---|---|---|---|---|---|---|---|---|---|---|---|
| | | | Methods | | | | Distal | Fetal | CD Differentiation | | |
| | Origin | | | Selection | | Branching | tip | Kidney | Principal Cell | PC | IC |
| Publication | Source | Species | Direct? | (Efficiency) | Morphology | (Bifurcation) | RET | Interaction | Identity | Function | Function |
| This | PSC | human | Yes | None | Tip-Stalk | Yes | +++ | Yes | AQP2 (IF, qPCR, | +++ | +++ |
| disclosure | | | | (~90%) | | | | | scRNA) | | |
| | | | | | | | | | ENaC (IF, qPCR, | | |
| | | | | | | | | | scRNA) | | |
| Taguchi⁶ | PSC | mouse | Yes | FACS^{a} | Tip-Stalk | Yes | + | Yes | n.d. | None | None |
| | | | | (<50%) | | | | | | | |
| | PSC | human | Yes | FACS^{a} | Tip-Stalk | +/− | n.d. | n.d. | n.d. | None | None |
| | | | | (<50%) | | (slow | | | | | |
| | | | | | | growth) | | | | | |
| Kuraoka⁷ | PSC | human | Yes | FACS^{a} | Tip-Stalk | +/− | +++ | n.d. | AQP2 (IF, | None | None |
| | | | | (<50%) | | (slow | | | qPCR) | | |
| | | | | | | growth) | | | | | |
| Zeng⁸ | Primary | mouse | n/a | n/a | Tip-Stalk | Yes | +++ | Yes | AQP2 (IF, | None | None |
| | | | | | | | | | qPCR) | | |
| | Primary | human | n/a | n/a | Diffuse | No | n.d. | n.d. | AQP2 (qPCR) | None | None |
| | | | | | growth | | | | | | |
| | PSC | human | Yes | FACS^{b} | Diffuse | No | − | n.d. | AQP2 (qPCR) | None | None |
| | | | | (55%) | growth | | | | AQP3 (IF, | | |
| | | | | | | | | | qPCR) | | |
| Mae⁹ | PSC | human | Yes | none | Diffuse | +/− | + | n.d. | AQP2 (IF, | None | None |
| | | | | | Some tip- | | | | qPCR) | | |
| | | | | | stalk | | | | | | |
| Howden¹⁰ | PSC | human | No^{c} | from | Diffuse | No | n.d. | Yes | Weak AQP2 | None | None |
| | | | | organoid | growth | | | | (scRNA) | | |

*Human PSC-based protocols highlighted in blue.
^{a}KIT/CXCR4-positive cells.
^{b}KIT-positive cells.
^{c}Derived indirectly from metanephric-like kidney organoids.

The sequential steps progress through the stages of ureteric epithelial development, as verified by extensive molecular characterization including scRNA-seq. The UB organoids manifest a complex three-dimensional morphogenetic trajectory, which parallels that of isolated rodent UBs grown in 3D culture¹⁴, including bifurcative branching with polarized tip-stalk organization. Correlating with their physiologic growth properties, the UB cells are competent to integrate into a chimeric developmental progenitor niche. The organoids differentiate into CD epithelia at >95% efficiency based on scRNA-seq clustering, and they represent the inner medullary CD at >85% efficiency using unbiased computational analyses. They contain AQP2/ENaC-expressing PCs, which exhibit a robust capacity for electrogenic sodium transport. In response to FOXI1 expression, the epithelia differentiate into ICs with V-type ATPase activity and proton secretion. Overall, this differentiation approach generates UB and CD tissues that recapitulate a range of features, including developmental stages, growth and morphology, cell fate determination, and ion transport physiology, thereby opening opportunities for diverse investigations and applications (some examples are summarized below).

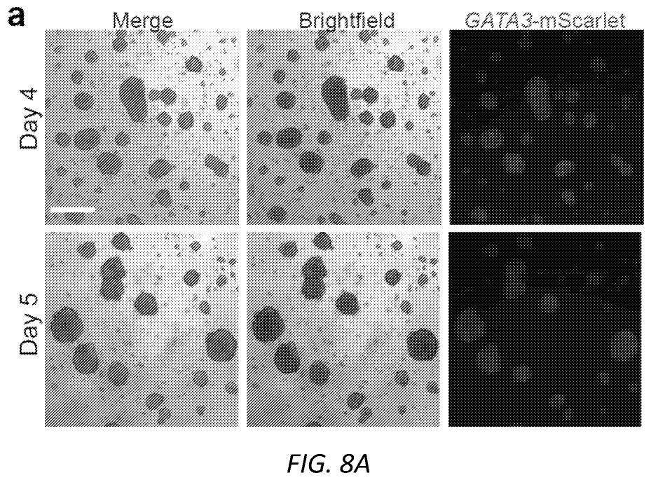

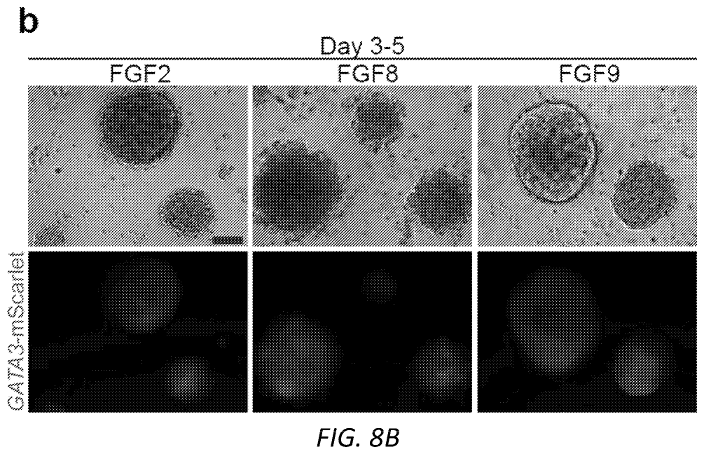

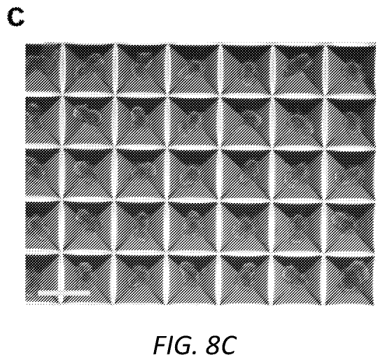

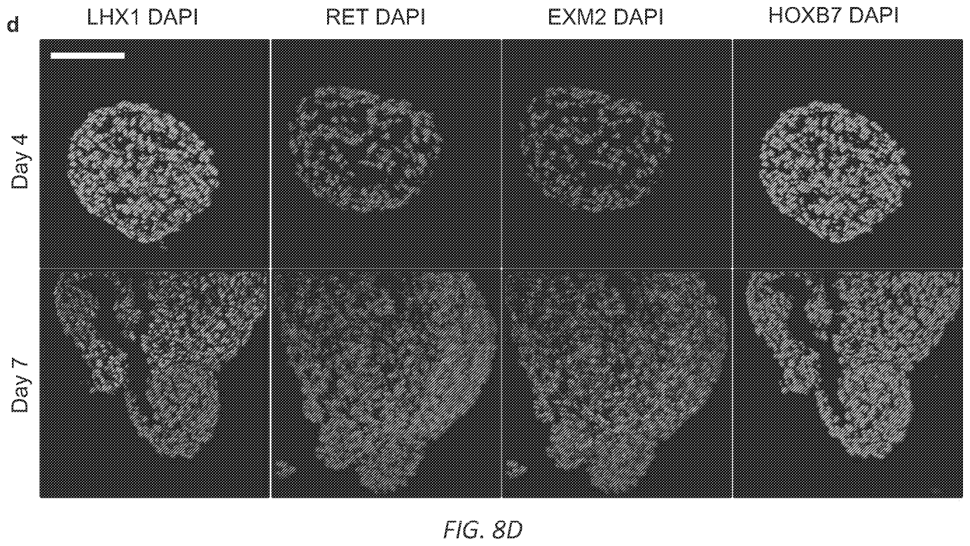

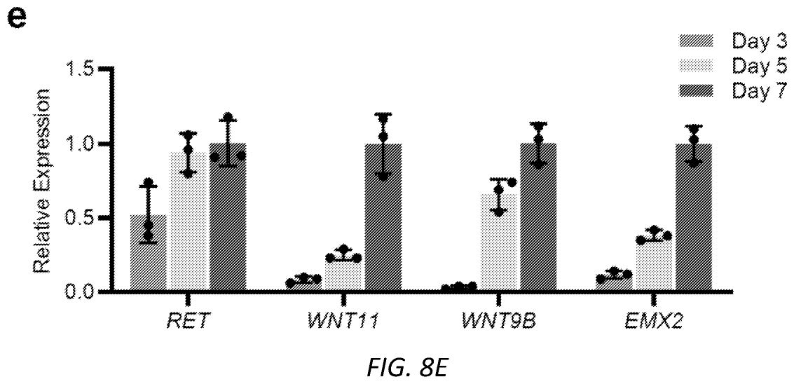

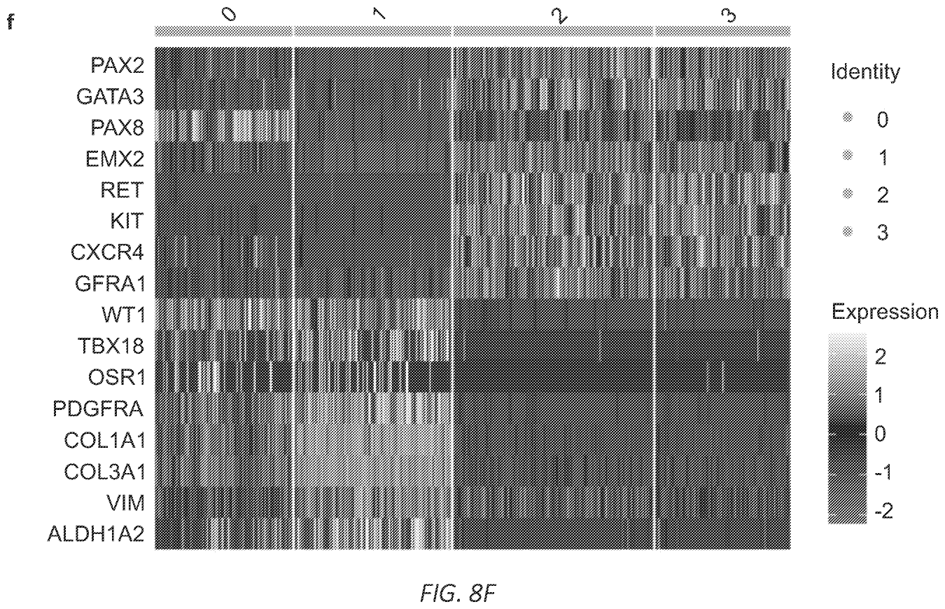

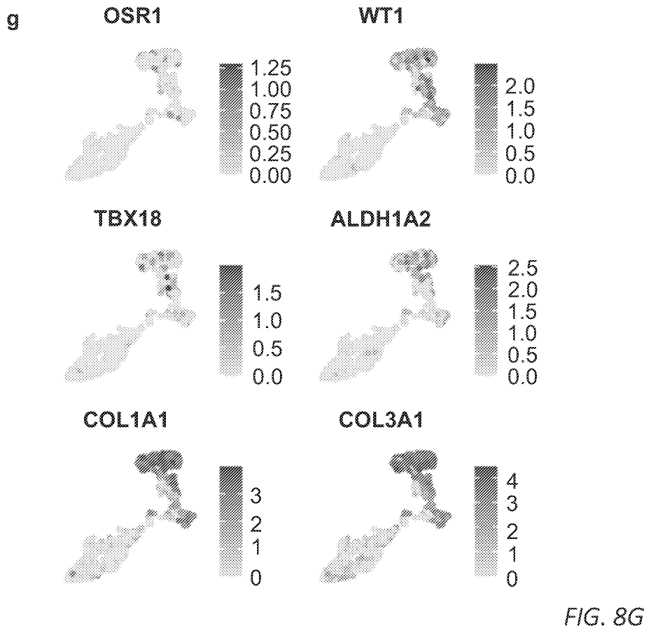

The protocol offers high efficiency, reproducibility, and relative ease of implementation. We carefully measured each of the early steps that lead to ND fate to enable straightforward optimization and application of the protocol. Previous methods reported low efficiencies of ND induction (18-46%⁷, ~40-60%⁶, and 36-55%⁸) and therefore required cell sorting, which may eliminate potentially important supporting populations, such as the stromal cells we described at day 7. Our approach successively induces mesendodermal and pronephric IM fates in the monolayer format with >95% TBXT- and >90% GATA3-positive cells, respectively, resulting in negligible off-target differentiation detected in the ND spheroids at day 7 and >95% CD cell types in the organoids at day 18. Our method is readily scalable, as progenitors treated as described herein (e.g., plated into a patterned microwell plate or at low density on a low attachment substrate at day 3) reliably form ~1,200 UB spheroids in a single well (FIG. 8c), with nearly 100% of the spheres exhibiting both the ND and stromal progenitor domains and the competence to form branching organoids. Scale-up using previous methods for propagating hPSC-derived UB progenitors required laborious manual microdissection of the individual tip domains for serial passage^{8, 9}. The present methods do not require cell sorting, microdissection, or purification in order to obtain UB or CD organoids.

The iterative branching of the UB is a key determinant of kidney architecture and nephron endowment, as perturbed branching leads to a spectrum of renal hypodysplasia including congenital birth defects in humans^{46, 47}. The UB organoids described here exhibit analogous branching behavior, in contrast to previous methods for in vitro propagation and expansion of hPSC-derived UB progenitor cells that produced rapidly growing epithelia but with a folded or ‘flowering’ phenotype with cells predominantly biased toward the tip fate and lacking dichotomous branching^{8-10.} Another strategy using embryoid body-based differentiation, cell sorting, and serum-containing growth medium generated UB organoids with good tip-stalk polarity and some evidence of terminal branching^{6, 7}, but rather static growth over several weeks of 3D culture. Our organoid model has the capacity for dynamic growth and bifurcative branching, as well as maintenance of the stereotypic tip-stalk morphology. The branching was somewhat disorganized as expected given the lack of organizing mesenchyme⁴⁸, but these structures will be valuable in future research on human kidney tissue engineering once they can be assembled with authentic metanephric-like progenitors. Previous attempts to mix metanephric and ureteric-like cells did not result in branching morphogenesis or other developmental interactions between the compartments^{36, 37}, but here we demonstrate that UB organoid-derived cells can incorporate durably in the UB tip compartment in an ex vivo developing kidney (FIG. 2f-h), which is compelling given that the cells had to compete across an interspecies boundary.

Despite recent progress in generating renal cell types from hPSCs, the derivation of cells with demonstrated physiologic kidney functions has remained elusive. Our organoid-derived PCs maintained in 2D culture demonstrate robust amiloride-sensitive vectorial sodium transport, which to our knowledge has not been reported in hPSC-derived renal epithelial cells or cultured human CD epithelium from any source. ENaC activity has been shown in immortalized mouse kidney cells¹¹ but not in immortalized human kidney cells¹². Our PCs also displayed appropriate responsiveness to aldosterone. The physiologic pathways recapitulated in the PCs are targets of two widely used drug classes (ENaC antagonists and mineralocorticoid receptor antagonists), testifying to the cells' potential in pharmacologic discovery and investigation.

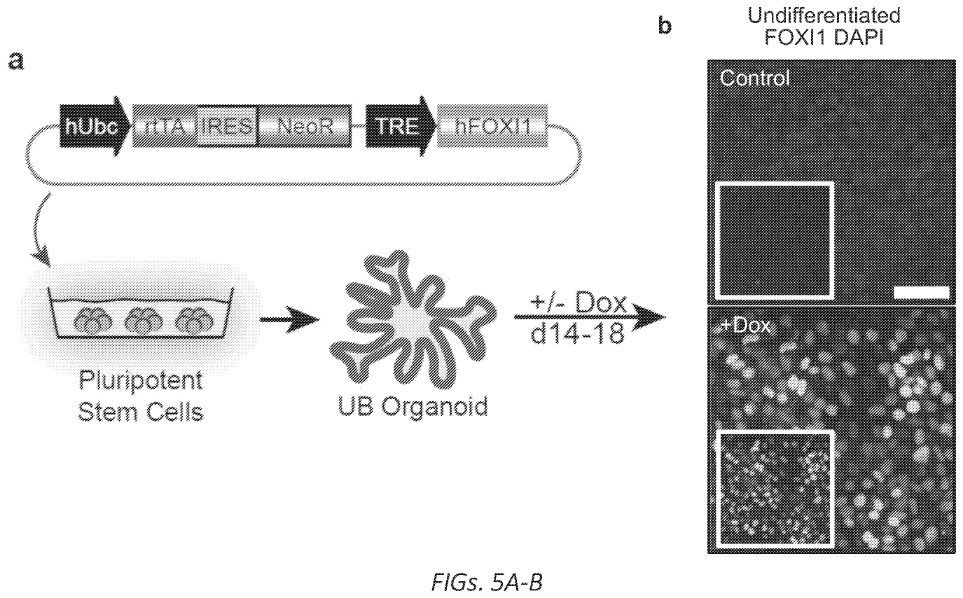

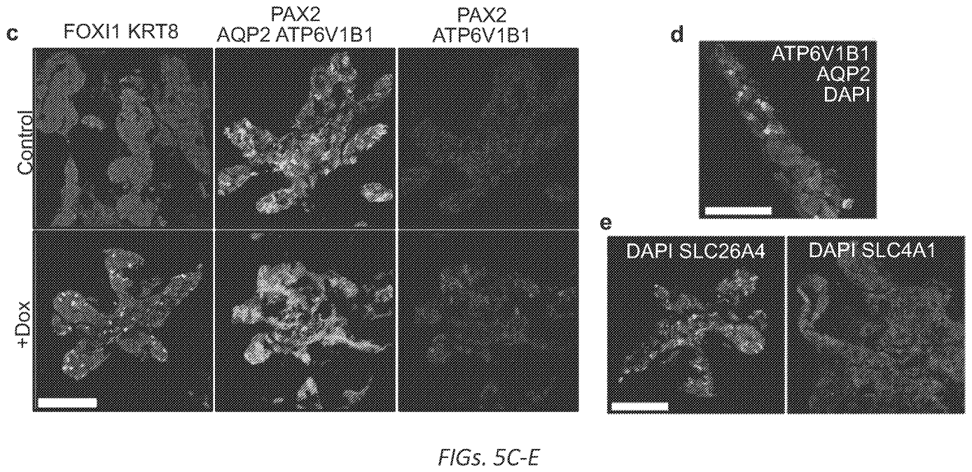

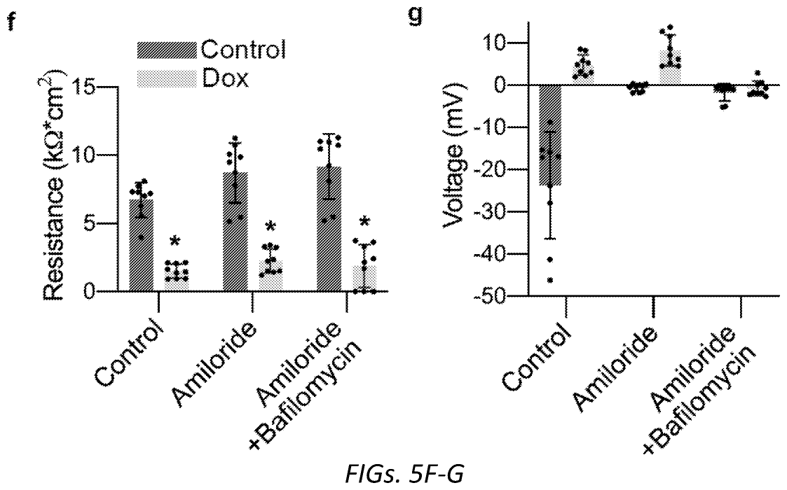

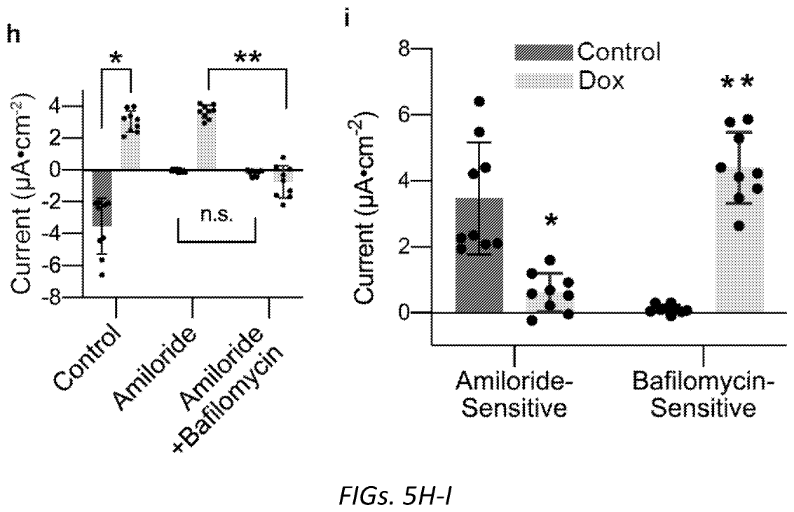

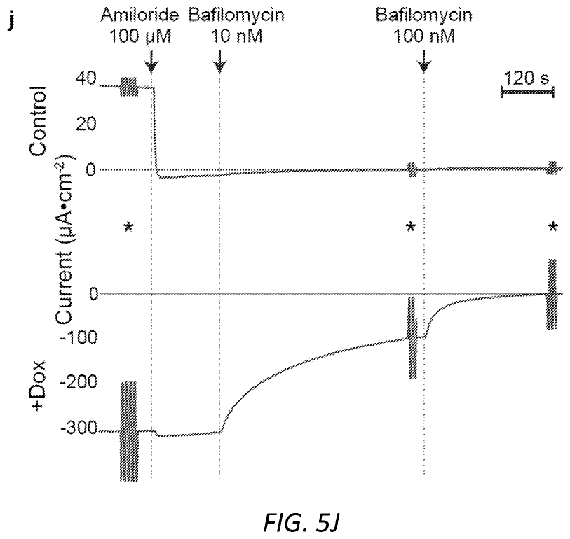

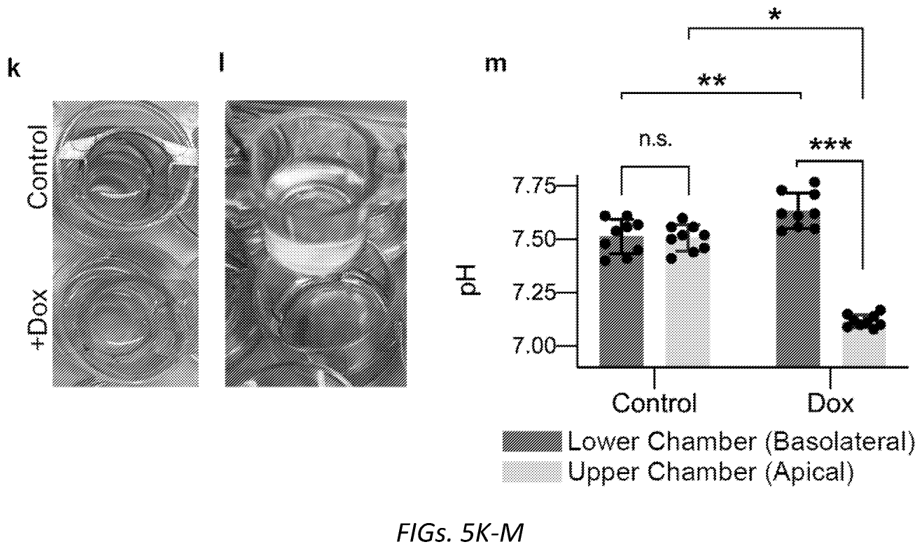

Nephrology has lacked in vitro models of ICs for experimental study, as even primary ICs isolated from rodent CDs rapidly lose their differentiated phenotype in culture¹³. Our protocol did not spontaneously yield ICs (FIG. 5d), which likely was related to the organoids' inner medullary phenotype since this region normally is devoid of ICs. It is also possible that the protocol lacks a necessary developmental cue since, in another system, organoids derived from hPSCs also did not form FOXI1⁺ ICs while organoids derived from primary mouse UB developed well differentiated ICs⁸. Nevertheless, we showed that exogenous FOXI1 expression reproducibly induced the IC fate, demonstrating that it is sufficient to promote specification of ICs and provides a method to reliably derive ICs in a controlled manner in vitro. The electrophysiologic properties of the sodium-reabsorbing PCs were converted into those of acid-secreting ICs more consistent with A-ICs that secrete protons across the apical membrane (FIG. 5k-l), as opposed to the B-IC fate induced in the 3D organoids (FIG. 5e). Although the underlying mechanisms remain unknown, this is in agreement with previous observations that primary isolated B-ICs rapidly drift toward an A-IC identity in 2D culture¹³. This phenomenon is likely related to the plasticity among IC subtypes^{49, 50}, which allows physiologic adaptation in response to the metabolic demands of an organism or its environment. Our approach may be helpful in dissecting the mechanisms governing IC fate determination. Moreover, it provides a culture system that exhibits IC-specific electrophysiology and ion transport to enable interrogation of novel IC biology and function.

Although the CD epithelium represents only a small fraction of cells in the kidney, it has important roles as the final site of physiologic modification of urinary composition and as the structural system for urinary drainage. It is also a rare example of a tissue in which the basic functions of ion transport and electrophysiology are tightly correlated with pathophysiology and disease states, including salt sensitivity and hypertension, electrolyte imbalance, water disequilibrium, kidney stones, and distal renal tubular acidosis. The differentiated cells presented here will aid investigation of both inherited and acquired disorders involving the CD and of putative novel drug targets. As one example, the lithium-induced side effects of nephrogenic diabetes insipidus and chronic kidney disease are of interest in psychiatry, but their precise pathobiological mechanisms remain unknown. In animal models, impaired urine concentrating ability results from a mis-regulated balance between PC and IC populations⁵¹. Our hPSC-derived CD organoids will permit modeling of the impact of lithium on fate decisions between these two cell types.

Described herein are efficient methods for derivation of UB and CD organoids from hPSC that progresses through the normal developmental stages and morphologic processes. The CD organoids efficiently form differentiated PCs and exhibit competence to induce ICs in response to FOXI1 expression. We have also used the organoids to derive a 2D cell line with similar cytodifferentiation characteristics, which exhibits robust ENaC-dependent sodium transport at baseline and V-type ATPase activity following FOXI1-mediated conversion to an IC state.

### Methods of Use

The organoids described herein have a number of potential uses, including the following.

1. Polycystic kidney disease (PKD): The autosomal dominant form of PKD (ADPKD) is one of the most common monogenic diseases in humans and frequently leads to end stage kidney disease and the need for dialysis or transplantation. It is the collecting duct where the cysts characteristic of PKD develop. There is one approved therapeutic, Tolvaptan, but this therapy has significant limitations. The organoid and cellular systems of unique human functional collecting duct cells we have generated allow us to interrogate them as models for cystogenesis. We have generated stably transfected doxycycline-inducible polycystin 1 CRISPRi human embryonic stem cells where we can precisely define the cellular abnormalities leading to cyst formation and test candidate drugs or perform unbiased screens to identify new targets. These systems will also be amenable to studying the effects of nrf2 agonists on cyst formation.

2. Distal nephron sodium reabsorption inhibition to treat hypertension and sodium overload: This would lead to new non-steroidal mineralocorticoid therapeutics with little effects on potassium secretion and/or specific kaliuretic agents. The ability to measure electrogenic amiloride sensitive currents on collecting duct cells derived from the organoids and the stability of these physiological properties through multiple passages in culture will greatly facilitate screening approaches.

3. Interstitial Fibrosis and Chronic Kidney Disease: It is known that abnormalities of adhesion molecules as might occur in acute and chronic kidney disease in the collecting duct leads to the enhanced generation of TGFb and the production of collagen and fibronectin with increased apoptosis and proliferation of cells and our hypothesis is that the ensuing fibrosis is related to cellular senescence. The cellular 2D and 3D systems we have created will be amenable to interrogating small molecule approaches to treatment.

4. Congenital Abnormalities of the Kidney and Urinary Tract (CAKUT): This new human model system will permit the study of the factors important for disease presentations of the urinary tract using CRISPR screening approaches, e.g., with CRISPRi. This will then lead to strategies to prevent early developmental abnormalities or interventions which will prevent progression of these abnormalities. For example, one or more mutations can be introduced into the PSC before generation of a UB or CD organoid as described herein.

5. Bioengineered Systems for Screening, Efficacy and Toxicity Studies In Vitro: The organoids can be placed into a multiwell microfluidic system where they can be interrogated using many different perturbations at different stages of development. We can also use the collecting duct cells to populate tubule structures forming a monolayer so that they can mimic a collecting duct in vivo. To mimic the hemodynamics of the collecting system in vivo we can also engineer bifurcations similar to the branching structures seen in the FIG. 2A-B. When coupled to high sensitivity assay system we can measure pH and cytokine production. This will allow for development of sensitive assays for new drugs targeting the distal nephron. For example, flow conditions can be established in a collecting duct mimic and transepithelial Na+, K+ and water flux can be measured in the presence or absence of a test compound, e.g., to evaluate libraries of molecules to identify those with the ability to modulate electrolyte and water movement, e.g., in the presence or absence of hormones such as aldosterone or vasopressin. This could result, for example, in a drug that could inhibit aldosterone action without the side effects of hyperkalemia due to K+ retention. CD cells derived from organoids developed from iPSCs from individuals with genetic diseases affecting Na+ movement across the collecting duct such as Liddle's syndrome can be established and used to screen for inhibitors that would reduce Na+ reabsorption into the body, e.g., to identify therapeutic candidates to treat hypertension. Cell lines can also be established from individuals with polycystic kidney disease (PKD) due to various mutations and used to screen for molecules that prevent cyst formation by these cells, e.g., to identify therapeutic candidates to prevent cyst formation. iPSCs from individuals without the disease can have a disease causing mutation introduced by various gene editing approaches and then the CD cells could be generated from organoids and used in screening as described above.

6. Drugs to minimize Lithium toxicity. The site of Lithium toxicity is the principal cell of the collecting duct. The present systems allows for the testing and development of agents that can counteract the toxic effects of lithium, which result in kidney diabetes insipidus and tubulointersitial fibrosis and chronic kidney disease. Lithium is used for bipolar disorder, depression and schizophrenia. For example, since Lithium is known to cause loss of AQP2 expression in principal cells, moderate- to high-throughput chemical screens (such as in 96-well or 384-well plates) could be applied to identify potential drugs that ameliorate the loss of AQP2 (using either a genetic fluorescent reporter allele or antibody staining) induced by Lithium in induced principal cells obtained using a method described herein.

7. Source of collecting duct cell lines: These organoids can serve as a source of human collecting duct cell lines that can be used to study physiology and pathophysiology and develop drugs such as diuretics or agents to treat acidosis.

Included herein are methods for screening test compounds, e.g., polypeptides, polynucleotides, inorganic or organic large or small molecule test compounds, using the organoids and cells therefrom to identify agents useful in the treatment of disorders associated with renal dysfunction.

As used herein, “small molecules” refers to small organic or inorganic molecules of molecular weight below about 3,000 Daltons. In general, small molecules useful for the invention have a molecular weight of less than 3,000 Daltons (Da). The small molecules can be, e.g., from at least about 100 Da to about 3,000 Da (e.g., between about 100 to about 3,000 Da, about 100 to about 2500 Da, about 100 to about 2,000 Da, about 100 to about 1,750 Da, about 100 to about 1,500 Da, about 100 to about 1,250 Da, about 100 to about 1,000 Da, about 100 to about 750 Da, about 100 to about 500 Da, about 200 to about 1500, about 500 to about 1000, about 300 to about 1000 Da, or about 100 to about 250 Da).

The test compounds can be, e.g., natural products or members of a combinatorial chemistry library. A set of diverse molecules should be used to cover a variety of functions such as charge, aromaticity, hydrogen bonding, flexibility, size, length of side chain, hydrophobicity, and rigidity. Combinatorial techniques suitable for synthesizing small molecules are known in the art, e.g., as exemplified by Obrecht and Villalgordo, Solid-Supported Combinatorial and Parallel Synthesis of Small-Molecular-Weight Compound Libraries, Pergamon-Elsevier Science Limited (1998), and include those such as the “split and pool” or “parallel” synthesis techniques, solid-phase and solution-phase techniques, and encoding techniques (see, for example, Czarnik, Curr. Opin. Chem. Bio. 1:60-6 (1997)). In addition, a number of small molecule libraries are commercially available. A number of suitable small molecule test compounds are listed in U.S. Pat. No. 6,503,713, incorporated herein by reference in its entirety.

Libraries screened using the methods of the present invention can comprise a variety of types of test compounds. A given library can comprise a set of structurally related or unrelated test compounds. In some embodiments, the test compounds are peptide or peptidomimetic molecules. In some embodiments, the test compounds are nucleic acids.

In some embodiments, the test compounds and libraries thereof can be obtained by systematically altering the structure of a first test compound, e.g., a first test compound that is structurally similar to a known natural binding partner of the target polypeptide, or a first small molecule identified as capable of binding the target polypeptide, e.g., using methods known in the art or the methods described herein, and correlating that structure to a resulting biological activity, e.g., a structure-activity relationship study. As one of skill in the art will appreciate, there are a variety of standard methods for creating such a structure-activity relationship. Thus, in some instances, the work may be largely empirical, and in others, the three-dimensional structure of an endogenous polypeptide or portion thereof can be used as a starting point for the rational design of a small molecule compound or compounds. For example, in one embodiment, a general library of small molecules is screened, e.g., using the methods described herein.

In some embodiments, a test compound is applied to a test sample, e.g., an organoid or a cell from an organoid developed as described herein, and one or more effects of the test compound is evaluated.

Methods for evaluating effects are known in the art. For example, ability to modulate expression of a protein can be evaluated at the gene or protein level, e.g., using quantitative PCR or immunoassay methods. In some embodiments, high throughput methods, e.g., protein or gene chips as are known in the art (see, e.g., Ch. 12, Genomics, in Griffiths et al., Eds. Modern genetic Analysis, 1999, W. H. Freeman and Company; Ekins and Chu, Trends in Biotechnology, 1999, 17:217-218; MacBeath and Schreiber, Science 2000, 289 (5485): 1760-1763; Simpson, Proteins and Proteomics: A Laboratory Manual, Cold Spring Harbor Laboratory Press; 2002; Hardiman, Microarrays Methods and Applications: Nuts & Bolts, DNA Press, 2003), can be used to detect an effect on expression of AQP2 or other markers of renal development or function.

The following Table 2 provides additional possible features and applications. The applications can be generally applicable beyond the specific feature noted.

TABLE 2
Summary of examples of potential applications.

| Feature | Applications | |
|---|---|---|
| High differentiation | 1. | Scalable production UB progenitors |
| efficiency | 2. | Easy for other labs to adopt and utilize methods |
| | 3. | Tissue engineering of collecting ducts and/or |
| | | ureteric tissue |
| Robust branching | 1. | Gain mechanistic insights into regulation of |
| morphogenesis | | human UB branching and patterning |
| | 2. | Model congenital anomalies of the kidney and |
| | | urinary tract (CAKUT) |
| | | a) Monogenic causes |
| | | b) Teratogens (i.e. ACE inhibitors) |
| | 3. | Tissue engineering of complex kidney organoids |
| | | based on UB interactions with metanephric cells |
| Collecting duct development, | 1. | Understand basic mechanisms of formation of |
| cell fate decisions, and | | radial polarity (corticomedullary axis) |
| cytodifferentiation | 2. | Interrogate process of principal cell maturation |
| | 3. | Identify signals and pathways involved in PC vs |
| | | IC fate determination (also Lithium-induced DI) |
| Functional Principal Cells | 1. | Interrogation of physiology and homeostasis: |
| | | a) Cell biology of ion transport |
| | | b) Mechanisms of hormone regulation |
| | | c) Develop assays for potassium secretion |
| | 2. | Disease Modeling: |
| | | a) Salt sensitivity/primary hypertension |
| | | b) Monogenic hypertension (e.g. Liddle) |
| | | c) Electrolyte wasting disorders |
| | | (pseudohypoaldosteronism) |
| | | d) Water disequilibrium |
| | | (Lithium-induced nephrogenic DI) |
| | | (Congenital nephrogenic DI) |
| | | 3. Study effect of potential pharmacologic agents on |
| | | different aspects of principal function |
| | | 4. Engineering 3D perfusable CD tubules for |
| | | modeling physiology and for regenerative medicine |
| Functional Intercalated Cells | 1. | Basic IC development and physiology |
| | | a) A-IC vs B-IC determination/polarity |
| | | b) Mechanisms of acid/base secretion |
| | 2. | Disease Modeling: |
| | | a) Distal renal tubular acidosis |
| | | b) Kidney stone disorders |
| | 3. | Identify candidate molecules that impact urine |
| | | acidification or alkalinization. |

### EXAMPLES

The invention is further described in the following examples, which do not limit the scope of the invention described in the claims.

### Methods and Materials

The following materials and methods were used in the Examples below.

### Pluripotent Stem Cell Culture

The H9 (WA09) hESC line was obtained from Wicell. The BJFF.6 hiPSC line was kindly provided by Sanjay Jain (Washington University, MO). H9 hESC and BJFF.6 hiPSC were maintained in feeder-free conditions on hESC-qualified Matrigel (Corning, catalog no. 354277) in mTeSR1 media (Stem Cell Technologies, catalog no. 05850) using 6-well tissue culture plates (Falcon, catalog no. 353046) in a 37° C. incubator with 5% CO2. Colonies were routinely passaged at a 1:8 split ratio every four days using Gentle Cell Dissociation Reagent (Stem Cell Technologies, catalog no. 07174) according to the manufacturer's protocol. Studies involving hESCs were reviewed and approved by Mass General Brigham Institutional Biosafety Committee (2011B000287).

### Mouse Experiments

Mouse experiments and housing were performed according to the animal use protocol approved by the Institutional Animal Care and Use Committee of Brigham and Women's Hospital (Protocol 2016N000162). Timed pregnant dams of CDI background were purchased from Charles River and arrived on the day of experiment. Animals were sacrificed using a euthanasia chamber with carbon dioxide.

GATA3-mScarlet Reporter hESC Line Generation

To generate the donor plasmid, the left and right homology arms flanking the stop codon were amplified by high fidelity PCR (iProof, BioRad) from a template of genomic DNA isolated from H9 hESCs. The left homology arm (759 bp) forward and reverse primers were 5′-GGTAGAAGAGAGGCAACCGA-3′ (SEQ ID NO:1) and 5′-ACCCATGGCGGTGACC-3′ (SEQ ID NO:2); right homology arm (984 bp) primers were 5′-AGCCCTGCTCGATGCTC-3′ (SEQ ID NO:3) and 5′-GGCTGCAGGAATAGGGACAA-3′ (SEQ ID NO:4). These fragments were purified (PCR purification kit, Qiagen) and cloned into a pUC57 vector digested with NheI and SacI in a single reaction using HIFI cloning (New England Biolabs). For gRNA plasmid, oligos (5′-CACCGGCCCTGTGAGCATCGAGCA-3′ (SEQ ID NO:5) and 5′-aaacTGCTCGATGCTCACAGGGCC-3′ (SEQ ID NO:6)) were annealed and ligated into pX458 vector (Addgene 48138, kindly provided by Feng Zhang) digested with BbsI. All plasmid sequences were verified by Sanger sequencing (Genewiz).

Donor and Cas9/gRNA vectors were co-transfected (TransIT-LT1, Mirus) into H9 cells that were dissociated into single cells using Accutase (Stem Cell Technologies). After passaging, Hygromycin (50 μg ml-1) was added to the culture medium and cells were maintained under selection for one week. By day 4-5. individual resistant colonies emerged, which were expanded and genotyped. We used the following flanking forward and reverse primers: 5′-CTAGCGGAAGATTTTATGGCACC-3′ (SEQ ID NO:7) and 5′-CATATCGATTGGCCCGGGAT-3′ (SEQ ID NO:8) to generate an 863 bp product in correctly targeted clones. From one well of a 6-well plate we genotyped four clones, and three of which were correctly targeted and confirmed by Sanger sequencing. Clone #1 was subsequently used for differentiation experiments described herein.

Differentiation of hPSCs into Ureteric Bud (UB) Organoids

hPSCs were dissociated into single cells using Accutase (Stem Cell Technologies, catalog no. 07920) and plated into 24-well plates (Thermo, catalog no. 142475) at a density of roughly 30,000 cells/well in mTeSR1 with ROCK inhibitor Y-27632 (10 μM; Stem Cell Technologies). This resulted in ~30% starting confluency approximately 24 hours after plating. Cells were then differentiated into mesendoderm by adding 50 ng ml⁻¹Activin A (PeproTech), 25 ng ml⁻¹ BMP4 (PeproTech), 5 μM CHIR99021 (Cayman Chemical) and 25 ng ml⁻¹ FGF2 (PeproTech) in basic differentiation medium consisting of Advanced RPMI 1640 (Life Technologies) and 1× L-GlutaMAX (Life Technologies) for 30 hours. Subsequently, cells were differentiated to IM by exposure to 1 μM A83-01 (Cayman Chemical), 25 ng ml-1 FGF2, 0.1 μM LDN193189 (LDN; Cayman) and 0.1 μM Retinoic acid (RA; Sigma Aldrich) for two days in Advanced RPMI 1640 supplemented with 1× L-GlutaMAX. Media was changed every day.

To induce ND, IM cells were dissociated with Accutase, pelleted, and resuspended in differentiation medium supplemented with 50 ng ml-1 FGF9 and 0.1 UM RA, and plated in 96-well, round bottom, ultra-low attachment plates (Corning, catalog no. 7007) at 2-3×10⁴ cells per well. The plates were centrifuged at 100×g for 15 seconds and cultured at 37° C., 5% CO₂ for 2 days. This resulted in the formation of three-dimensional ND spheroids. At day 5, the half-medium change was performed using basic differentiation medium supplemented with 50 ng ml⁻¹ GDNF (Peprotech) and 0.1 μM RA. As an alternative to using 96-well plates, we demonstrated the ability to plate the cells onto patterned microwells (AggreWell-400, Stem Cell Technologies) at day 3 following dissociation. For 24-well AggreWell plates, approximately 1.0×10⁶ cells were plated into a single well according to manufacturer's instructions.

On day 7 of differentiation, spheroids were embedded into 100% Matrigel Matrix (Corning, catalog no. 354234) and plated as a 45 μl droplet in 24-well plates (Thermo, catalog no. 142475). The matrigel was allowed to solidify for at least 30 minutes in the tissue culture incubator, then was overlayed with basic differentiation medium supplemented with 50 ng ml-1 GDNF, 50 ng ml-1 FGF10 (Peprotech), 2 μM CHIR, 0.1 μM LDN, 1 μM A83-01, 0.1 μM RA and 2-5 μM U0126 (Cell Signaling Technologies) for 7-10 days. The medium was replaced every 3-4 days as needed. In many instances, we found that NDs derived from BJFF.6 cells could be transitioned to 3-D matrix as early as day 6 rather than waiting until day 7, whereas the H9-derived structures failed to develop if transitioned to Matrigel prematurely.

### Collecting Duct (CD) Organoid Formation

To induce differentiated CD cell types, the organoids were transitioned to basic differentiation medium supplemented with 10 nM arginine vasopressin (AVP) and 10 nM aldosterone for 4 days to generate CD organoids. For the experiments shown in the publication, this transitioned was done at day 14 and organoids were culture for four additional days. However, we have found that this differentiation process also works with comparable efficiency when done at later stages and for as short as 2-3 days.

### Passaging and Expansion of UB Organoids

While organoids that were kept under either control or differentiation conditions exhibited minimal growth potential beyond day 18-20, we found that addition of FGF7 (50 ng ml-1; Peprotech) to the UB progenitor media at day 14 induced continued growth and expansion of the epithelium. Under these conditions, UB organoids lost their tip-stalk organization and grew as folding epithelial sheets. Between days 24-28, we dissociated organoids directly in Matrigel using Accutase. Incubation at 37° C. for 10-15 minutes with frequent agitation by pipetting through P1000 pipet tip resulted in small epithelial fragments. Longer incubation ultimately led to a single cell suspension. We washed the fragments in PBS (1×) and pelleted them by centrifugation at 1,000×g for 3 minutes. Media and PBS were aspirated, 568 and the fragments were re-suspended in Matrigel and plated into a 24-well plate as detailed above. Typically, one well of day 24 UB organoids could be passaged into up to 20 wells. After allowing Matrigel to solidify for 30 minutes, UB progenitor media (AdRPMI with CHIR, GDNF, FGF10, FGF7, RA, A83, and LDN) was added to wells. Media was changed every 4-5 days. Organoids were passaged every 2-3 weeks. While we performed a majority of experiments with passaging of small epithelial fragments, we also observed that single cells could be similarly plated and expanded with the addition of ROCK inhibitor Y27632 (10 μM) to the media for the first four days after plating.

While control UB organoids exhibited little growth and branching beyond day 18, addition of FGF7 to the culture media at day 14 led to continuous proliferative expansion of GATA3-expression epithelial structures that lost tip-stalk organization. Under this condition, organoids were enzymatically dissociated into small fragments and passaged by re-embedding into fresh Matrigel matrix. The expandable UB tissues (day 28) expressed progenitor markers including PAX2 and GATA3. The tip marker RET was observed in large areas of epithelium but was heterogeneously expressed. There was minimal or only very weak expression of the principal cell marker AQP2. Micrographs of expanded UB organoids at passage 3, day 21 (day 65 in total) demonstrated maintenance of GATA3 expression and folding morphology that is characteristic of these passaged organoids. True branching morphogenesis was not observed beyond the initial stages of differentiation (days 7-18). In four individual experiments, we observed continued growth and expansion potential of UB tissue for at least 3-7 passages. In each case, experiments were terminated voluntarily rather than due to failure of the UB organoids to proliferate.

### Chimeric Aggregation Assay

To assess the potency of the UB organoid tissue, we used re-aggregation techniques that have been previously described⁵² to generate chimeric kidney explants. Mouse embryos were isolated from timed pregnant females (Charles River) at E12.5 (day of plug=0.5), and the entire urogenital systems were dissected and placed in DMEM (Corning Cellgro, catalog no. 10-013-CM). Kidneys were then isolated by manual dissection and dissociated into single cells by incubation in a 20 μl drop (4-6 kidneys per drop) of TrypLE at 37° C. for 4 minutes. The enzyme was then quenched by adding 50 μl of Kidney Culture Media (KCM; DMEM, Pen/Strep, 10% fetal bovine serum) and incubating at 37° C. for 10 minutes for recovery. The digested rudiments were then pooled in a 1.7 ml microcentrifuge tube with an additional 500 μl KCM, triturated using a P200 pipette, and counted. Simultaneously, UB organoids at day 11 were isolated from Matrigel and dissociated to single cells as described below (see section on scRNA-seq preparation). Aggregation was performed by mixing 90,000 fetal mouse kidney cells with 10,000 differentiated human UB cells in a microcentrifuge tube, and then the cells were pelleted at 700×g for 5 minutes. The chimeric cell pellet was then picked up and placed onto a Nucleopore Track-Etch Membrane filter disk (Whatman, pore size 1 μm). The filter was floated on 1 ml KCM in a 24-well tissue culture plate and incubated for 72 hours.

### UB Organoid Dissociation, Single Cell Capture, and Sequencing

For scRNA-seq, organoids were extracted from the Matrigel matrix using Cell Recovery Solution (Corning) on ice for 10-20 minutes, with intermittent pipetting using a P1000 pipette until the organoids were freely floating. We transferred the suspension to a 15 mL tube, allowed the organoids to sink by gravity, and aspirated the Cell Recovery Solution. After washing once with PBS, the organoids were incubated in TrypLE Express (Thermo Fisher) at 37° C. for 10-12 minutes, at which point they dissociated into single cells. For day 7, the ND spheroids were directly collected, washed, and incubated in TrypLE Express for 7-10 minutes. The single cell suspensions were passed through a 70 μm reversible strainer (Stem Cell Technologies, catalog no. 27260) and pelleted via centrifugation at 400×g for 3 minutes.

For multiplexing, samples were labelled using BD Human Single Cell Sample Multiplexing Kit (catalog no. 633781), pooled, and delivered to the Single Cell Core (HMS) for library preparation. Single cells were captured and libraries prepared using the BD Rhapsody system with the Whole Transcriptome Analysis (WTA) Amplification Kit (BD Biosciences, catalog no. 633801). All cells were loaded and captured on a single cartridge. The WTA and Sample Tag libraries were amplified and purified according to manufacturer's protocol. The libraries were pooled at a ratio such that the Sample Tag library composed ~3%, and then sequenced by the HMS Biopolymers Facility using Illumina NextSeq 550 with High Output kit, yielding a depth of 25,000 reads/cell.

Analysis of scRNA-Seq Data

Raw sequencing data were processed using the BD Rhapsody Complete Analysis Pipeline on the Seven Bridges Genomics cloud platform, which resulted in de-multiplexed counts matrices of gene expression in single cells. The R-package Seurat (v4.0.4) was used for downstream analyses including quality control, data normalization, data scaling, and visualization. Cells that expressed less than 200 genes, greater than 8,000 UMI, greater than 30% of reads assigned to mitochondrial genes, or definitive multiplets with two distinct sample tags were filtered out of the analysis. The final dataset contained 609 and 4,095 cells in the day 7 and day 18 organoid, respectively. A principal component analysis was used for dimension reduction with a dimension value of 18 determined by the JackStrawPlot function. The top 2,000 variable genes were selected and used together with dimensional information for clustering. Unsupervised clustering was performed and Uniform Manifold Approximation and Projection for Dimension Reduction (UMAP) plots were generated. For reference-based mapping, we uploaded the dataset representing day 18 organoids as an R object to the human kidney application on the Azimuth web app (azimuth.hubmapconsortium.org). The R package Monocle (v2.20.0) was used to perform cell lineage trajectory analysis on a randomly down-sampled (500 cells) subset of the principal cells from day 18.

### Inducible FOXI1 Expression for Specification of Intercalated Cells

cDNA for human FOXI1 was synthesized (Genewiz) and cloned into pDONR221 using Gateway cloning to generate an entry vector. The cDNA was shuttled into pInducer20-Blast (Addgene 109334, kindly provided by Jean Cook) recombination with LR Clonase II (Invitrogen). This plasmid was co-transfected with packaging and envelope plasmids (psPAX2, pMD2.G) into 293T/17 cells (ATCC), and lentiviral particles were harvested after 24 and 48 hours post-transfection.

H9 cells were passaged as single cells using Accutase with Y27632 and exposed to lentiviral supernatant for six hours. At six hours, media was changed with fresh mTeSR1. Two days following transduction, Blasticidin (10 μg ml-1; Invivogen) was added to the culture medium and cells were selected for four days. This cell line was differentiated into UB and CD organoids as described above. At day 14, the CD differentiation medium was supplemented with doxycycline (0.5 μg ml-1; Sigma) to activate expression of FOXI1.

RNA Isolation and qRT-PCR

Total RNA was isolated using Direct-zol RNA Miniprep (Zymoresearch, catalog no. R2051). 50-200 ng of RNA was used for reverse transcription with iScript cDNA synthesis kit (Bio-Rad, catalog no. 1708891) according to the manufacture's protocol. qRT-PCR was performed on iQ5 Multicolor Real-Time PCR Detection System (Bio-Rad) using iTaq Universal SYBR Green Supermix (Bio-Rad). Relative mRNA expression levels were analyzed by the ΔΔCT method and normalized to GAPDH gene expression. Primer sequences are listed in Table 3.

TABLE 3
QPCR primer sequences

| Gene | Forward primer | # | Reverse primer | # |
|---|---|---|---|---|
| AQP2 | CACGTCTCCGTTCTCCGAG | 9. | CTGTTGCTGAGAGCATTGACA | 10. |
| CDH1 | CGAGAGCTACACGTTCACGG | 11. | GGGTGTCGAGGGAAAAATAGG | 12. |
| ELF5 | TAGGGAACAAGGAATTTTTCGGG | 13. | GTACACTAACCTTCGGTCAACC | 14. |
| EMX2 | CGGCACTCAGCTACGCTAAC | 15. | CAAGTCCGGGTTGGAGTAGAC | 16. |
| EYA1 | GTCACAGTCTCAGTCACCTGG | 17. | GGGATAAGACGGATAGTCCTGC | 18. |
| FOXF1 | CCCAGCATGTGTGACCGAAA | 19. | ATCACGCAAGGCTTGATGTCT | 20. |
| GAPDH | CCCATCACCATCTTCCAGGAG | 21. | CTTCTCCATGGTGGTGAAGACG | 22. |
| GATA3 | GCCCCTCATTAAGCCCAAG | 23. | TTGTGGTGGTCTGACAGTTCG | 24. |
| HNF1B | TCAGCTGCTGTTTCTCTTTCCA | 25. | GCGGCGCATCTTCTTGTT | 26. |
| HOXA11 | TGCCAAGTTGTACTTACTACGTC | 27. | GTTGGAGGAGTAGGAGTATGTCA | 28. |
| HOXB7 | AACTTCCGGATCTACCCCTG | 29. | CTTTCTCCAGCTCCAGGGTC | 30. |
| LHX1 | CCTGGACCGCTTTCTCTTGAA | 31. | ACCGAAACACCGGAAGAAGTC | 32. |
| MIXL1 | GGCGTCAGAGTGGGAAATCC | 33. | GGCAGGCAGTTCACATCTACC | 34. |
| NANOG | TGATTTGTGGGCCTGAAGAAA | 35. | GAGGCATCTCAGCAGAAGACA | 36. |
| OSR1 | CCCATTTCGGTAGTTGCAGT | 37. | CCTTCAGCTAAAGCCCCAG | 38. |
| PAX2 | TGTCAGCAAAATCCTGGGCAG | 39. | GTCGGGTTCTGTCGTTTGTATT | 40. |
| PAX8 | AAGTGCAGCAACCATTCAACC | 41. | CTGCTCTGTGAGTCAATGCTTA | 42. |
| RET | ACACGGCTGCATGAGAACAA | 43. | GCCCTCACGAAGGGATGTG | 44. |
| SCNN1B | AGACAACCACAATGGCTTAACA | 45. | TGAGGCTACATAGTCTCATGGC | 46. |
| SCNN1G | GCACCCGGAGAGAAGATCAAA | 47. | TACCACCGCATCAGCTCTTTA | 48. |
| SIX2 | AAGGCACACTACATCGAGGC | 49. | CACGCTGCGACTCTTTTCC | 50. |
| SOX17 | GTGGACCGCACGGAATTTG | 51. | GGAGATTCACACCGGAGTCA | 52. |
| SOX9 | AGCGAACGCACATCAAGAC | 53. | CTGTAGGCGATCTGTTGGGG | 54. |
| TBXT | TATGAGCCTCGAATCCACATAGT | 55. | CCTCGTTCTGATAAGCAGTCAC | 56. |
| WNT11 | GACCTCAAGACCCGATACCTG | 57. | TAGACGAGTTCCGAGTCCTTC | 58. |
| WNT7B | GAAGCAGGGCTACTACAACCA | 59. | CGGCCTCATTGTTATGCAGGT | 60. |
| WNT9B | TGTGCGGTGACAACCTCAAG | 61. | ACAGGAGCCTGATACGCCAT | 62. |
| WT1 | GTGACTTCAAGGACTGTGAACG | 63. | CGGGAGAACTTTCGCTGACAA | 64. |

# SEQ ID NO:

### Immunofluorescent Staining

Cells cultured on coverslips were fixed in 4% paraformaldehyde for 45 minutes at room temperature (RT) and washed three times in PBS. For spheroids or organoids, the tissues were fixed in 4% paraformaldehyde for one hour at RT and thoroughly washed in PBS. Then tissues were mounted in OCT compound (Fisher Scientific), frozen in blocks, and cut into 7 μm sections. The sections were stored in-80° C. For staining, cells or slides were incubated in blocking buffer (0.1% Triton X-100 and 5% normal donkey serum in PBS) for one hour at RT, and incubated with primary antibody overnight at 4° C. in blocking buffer. They were then washed three times in PBS and incubated with secondary antibody (dilution 1:500) and DAPI (Sigma) for one hour at RT. Secondary antibodies (Jackson ImmunoResearch Laboratories) were made in donkey and conjugated to Alexa Fluor 488, 594, or 647. A list of primary antibodies is shown in Table 4. After staining, slides were mounted with Fluoromount G (Invitrogen) and air-dried for a minimum of several hours at RT. Imaging was performed using confocal microscopy (Nikon C1, Tokyo, Japan). Quantification was performed using Image-J by counting random fields at 400× magnification.

TABLE 4
Primary antibodies

| Antigen | Host species | Manufacture | Cat. No. | Dilution |
|---|---|---|---|---|
| AQP2 | Mouse | Santa Cruz | sc-515770 | 200 |
| AQP2 | Rabbit | Millipore | 178612 | 200 |
| ALDH1A3 | Rabbit | Novus | NBP2-15339 | 200 |
| ATP6V1B1 | Rabbit | Sigma | HPA031847 | 200 |
| CDH1 | Mouse | BD Biosciences | 610181 | 500 |
| EMX2 | Rabbit | Sigma | HPA065294 | 200 |
| FOXI1 | Goat | Abcam | ab20454 | 200 |
| SCNN1B | Rabbit | Gift from L. Palmer | | 100 |
| GATA3 | Rabbit | Cell Signaling | D13C9 | 300 |
| HNF1B | Rabbit | Sigma | HPA002083 | 200 |
| GRHL2 | Rabbit | Sigma | HPA004820 | 200 |
| HOXB7 | Mouse | R&D Systems | MAB8040 | 200 |
| KRT8 | Mouse | Santa Cruz | sc-8020 | 100 |
| LHX1 | Rabbit | Abcam | ab229474 | 200 |
| Nuclei (hu) | Mouse | Novus | NBP2-34342 | 500 |
| PAX2 | Rabbit | BioLegend | PRB-276P | 200 |
| PAX2 | Goat | R&D Systems | AF3364 | 200 |
| PAX8 | Rabbit | Abcam | ab189249 | 400 |
| RET | Goat | R&D Systems | AF-1485 | 200 |
| SOX9 | Rabbit | Novusbio | NBP1-85551 | 200 |
| SLC26A4 | Rabbit | Sigma | HPA042860 | 200 |
| TBXT | Goat | R&D Systems | AF2085 | 200 |

For wholemount staining, 3-D organoids were fixed with 4% paraformaldehyde in PBS for one hour at RT and thoroughly washed in PBS. The organoids were then incubated in blocking buffer for one hour at RT, then incubated with primary antibodies in antibody dilution buffer overnight at 4° C. The organoids were then washed with PBS three times for 20~30 minutes each. The organoids were incubated with secondary antibodies and DAPI in antibody dilution buffer for 2 hours at RT, then washed with PBS three times for 30 minutes each. The organoids were moved on slides, flat-mounted with Fluoromount G, and coverslipped.

### Electrophysiological Measurements in Transwell System

CD organoid-derived cells were dissociated to single cells and transitioned to 2D transwell culture conditions previously established for mouse CD cell lines¹¹. Briefly, the cells were grown at 37° C. with 5% CO₂ in DMEM/F-12 media supplemented with insulin (5 mg ml⁻¹), apotransferrin (5 mg ml⁻¹), sodium selenite (60 nM), 1 triiodothyronine (1 nM), dexamethasone (10 nM), epithelial growth factor (10 ng ml⁻¹), and fetal bovine serum (2% v/v). Under these conditions, we established a cell line that exhibited similar epithelial morphology as the mouse CD cells. The cells were serially passaged using Trypsin-EDTA. For electrophysiology studies, CD cells were seeded on Corning Cell Culture Inserts (12 well format, 0.4 mm pore size PET track-etched membranes) at 100,000 cells per well. Transepithelial resistance and voltage was monitored using epithelial voltohm meter (World Precision Instruments, EVOM3) with the STX2-plus electrode. The short-circuit current across the epithelia was calculated using Ohm's law, Voltage=Current*Resistance. Amiloride (10 μM; Sigma) was added above the transwell and transepithelial voltage and resistance measurements were repeated after 5 minutes. Aldosterone (Sigma) was used at varying concentrations for 24 hours in the absence of dexamethasone. For FOXI1 experiments, bafilomycin-A1 (10 nM; Sigma) was added following addition of amiloride and measurements were repeated after another 5 minutes.

### Transepithelial Short-Circuit Current (I_{sc}) Ussing Chamber Recordings

CD cells were grown on 12 mM Snapwell inserts with 0.4 μM polycarbonate membranes (Corning) in CD Medium for 7-14 days to ensure confluency. Snapwell inserts were mounted in Ussing Chambers (Physiological Instruments VCC MC8) at the Harvard Digestive Disease Center Core at Boston Children's Hospital. Bath solutions of 120 mM NaCl, 25 mM NaHCO₃, 3.3 mM KH₂PO₄, 0.8 mM K₂HPO₄, 1.2 mM MgCl₂, 1.2 mM CaCl₂), and 10 mM glucose were added to the chambers with bubbling CO₂ to maintain a pH of 7.4. Snapwells were continuously clamped at 0 mV and I_{SC} was recorded using LabChart software. Amiloride (10-100 μM) and, bafilomycin-A1 (10-100 nM) were added to the apical chamber as indicated.

### Statistical Analysis and Reproducibility

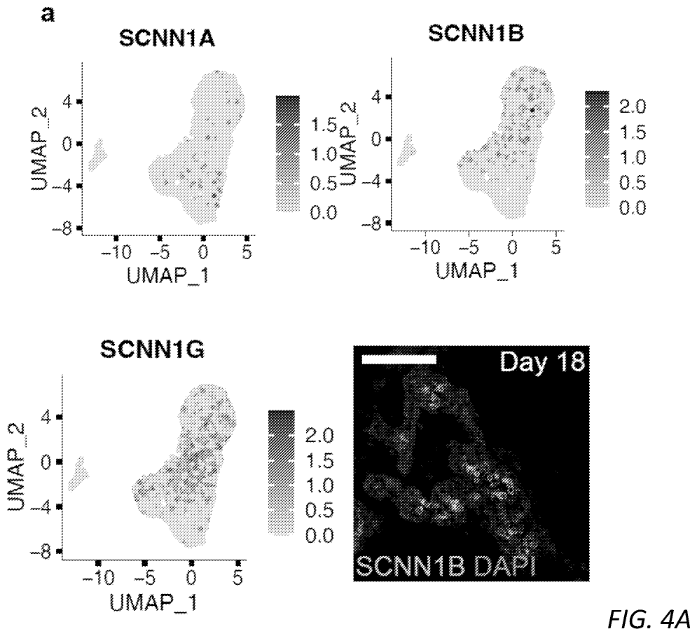

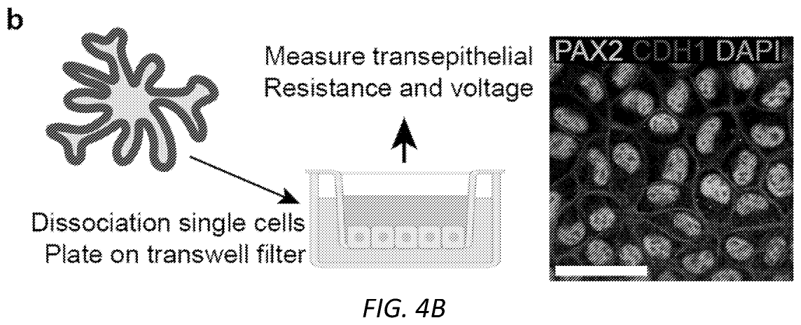

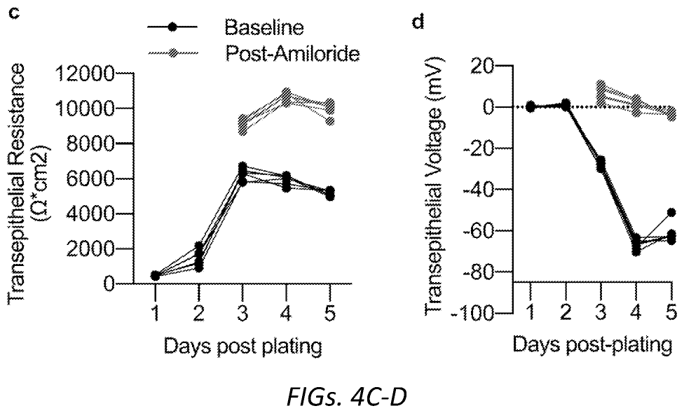

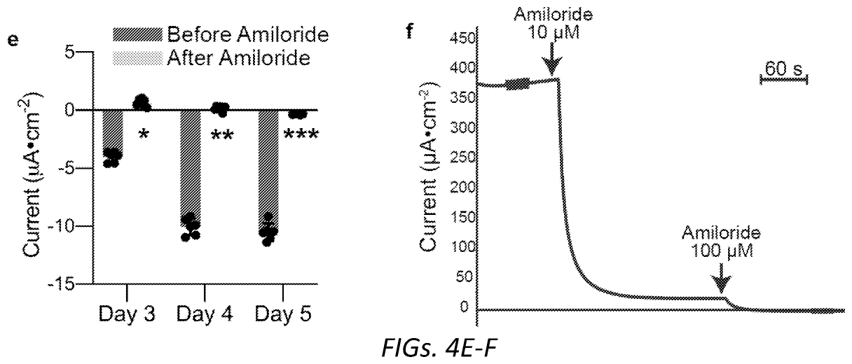

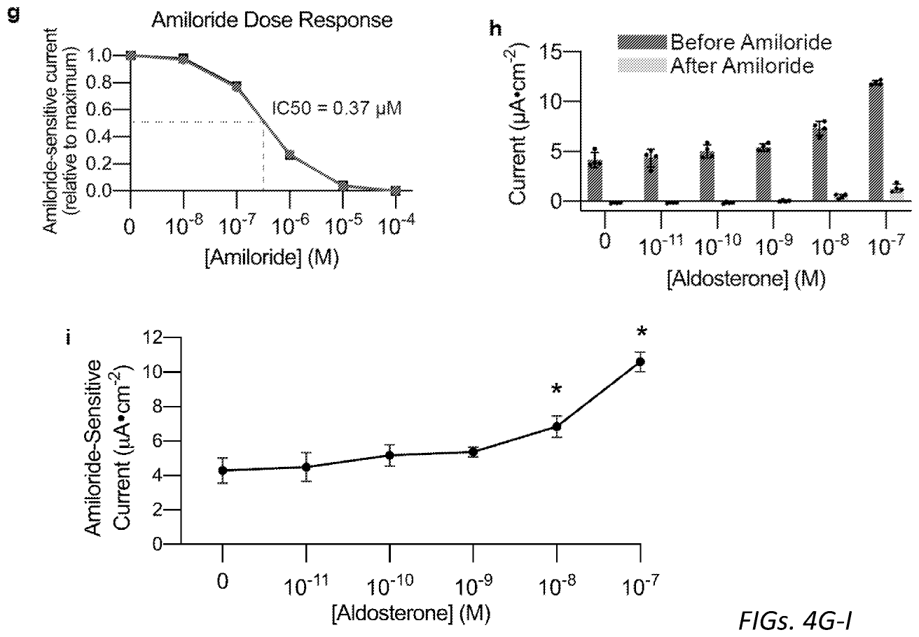

Values were presented as mean±standard deviation. Individual data points represent distinct samples rather than repeated measurements. All statistical tests performed were mentioned in figure legends. In brief, differences with values of p<0.05 were considered statistically significant. Sample sizes were provided in the figure legends. Two-tailed unpaired t-test (Student's t-test) assuming equal standard deviation was applied for statistical analysis of differences between two groups. Paired-sample t-test was used to compare the current results of induced-CD cells before and after amiloride treatment in FIGS. 4A-I. GraphPad Prism software 8 (GraphPad) was used to do statistical analysis. Statistical methods were not used to determine sample size. All p values were displayed in the figure legends.

### Example 1. Induction of Pronephric Mesoderm and ND Spheroids

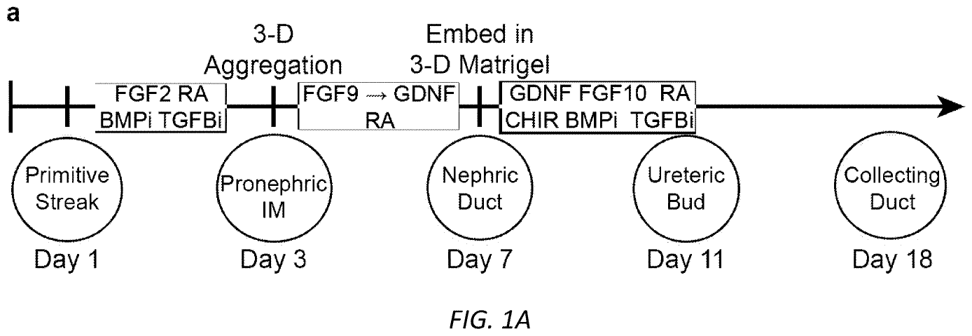

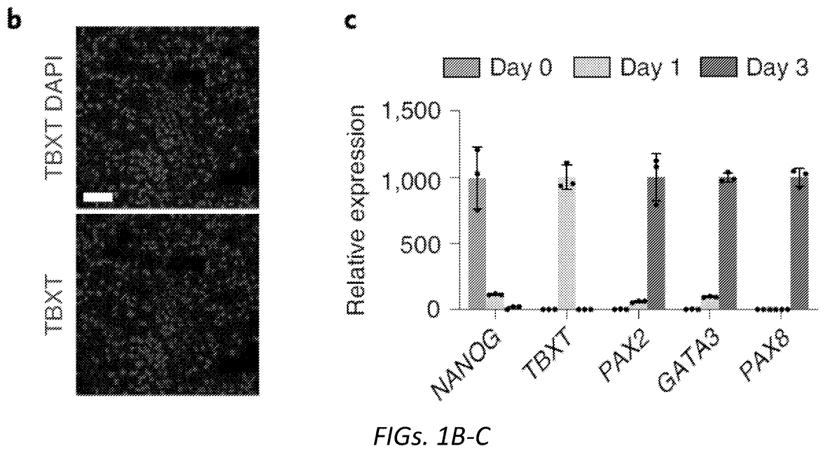

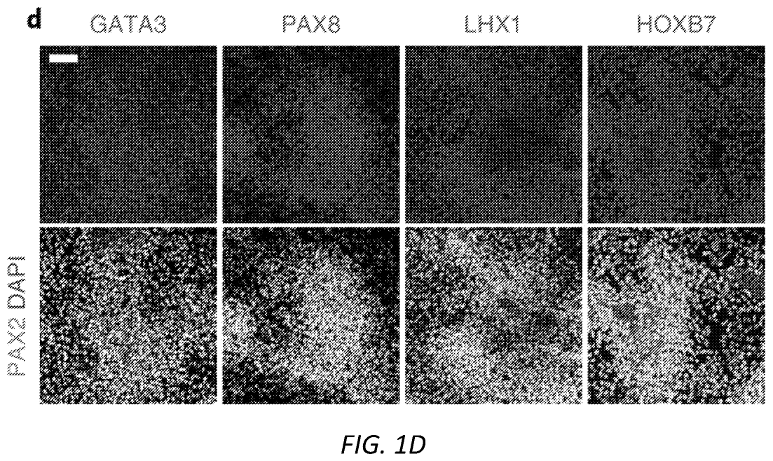

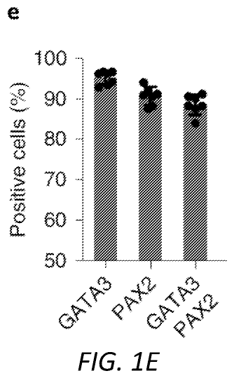

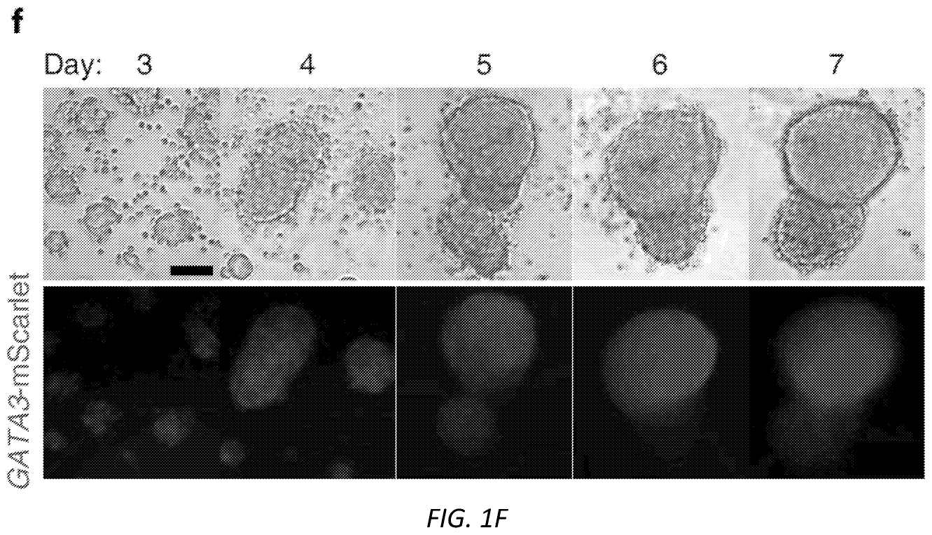

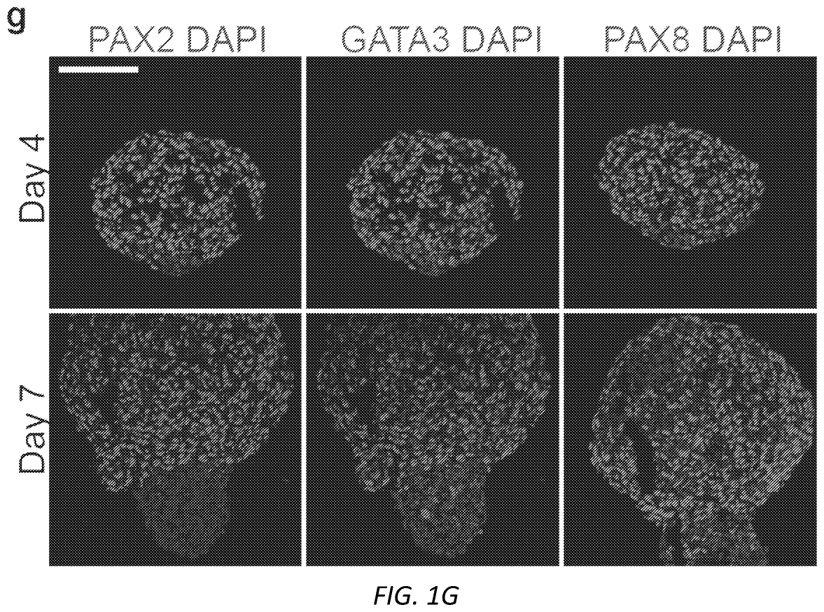

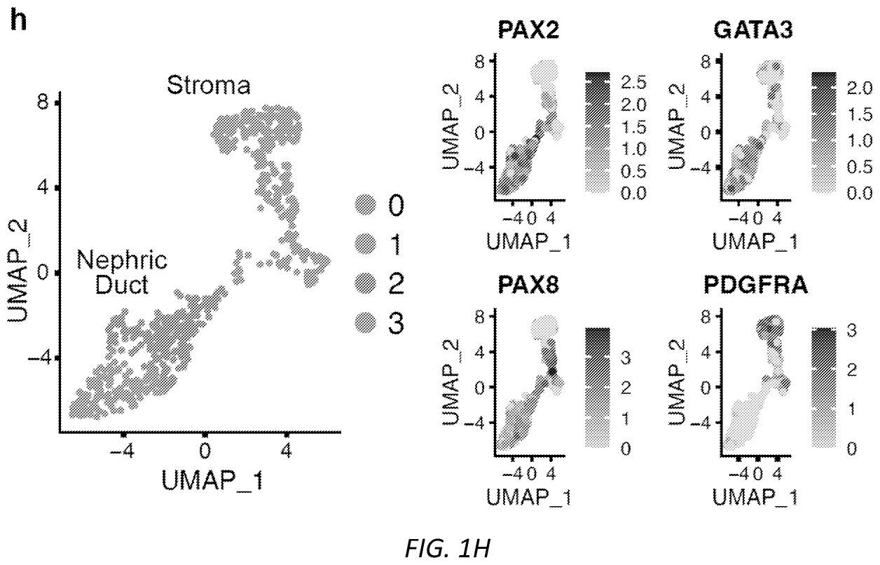

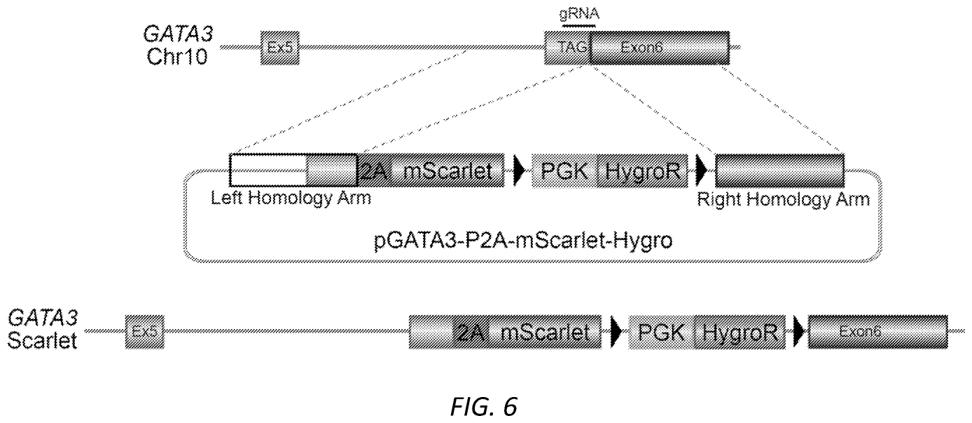

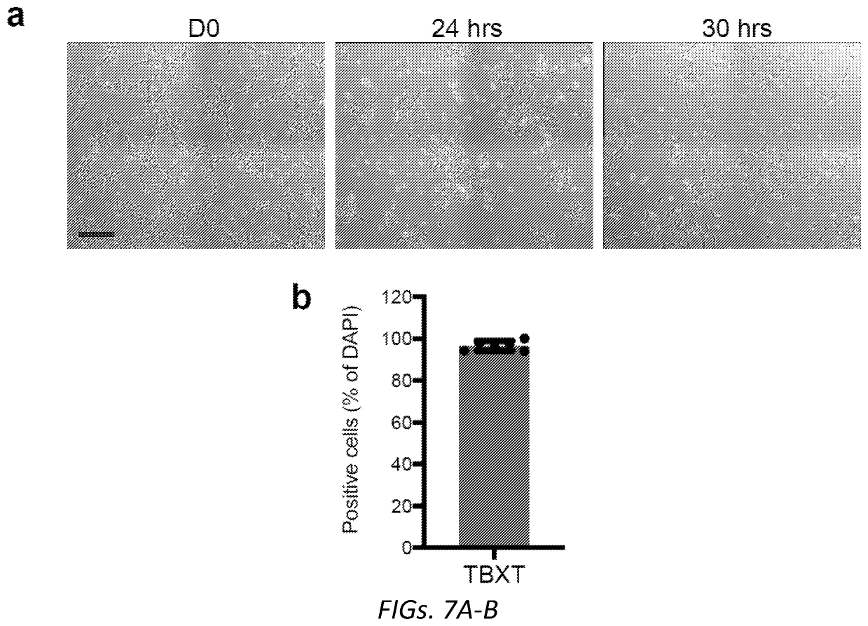

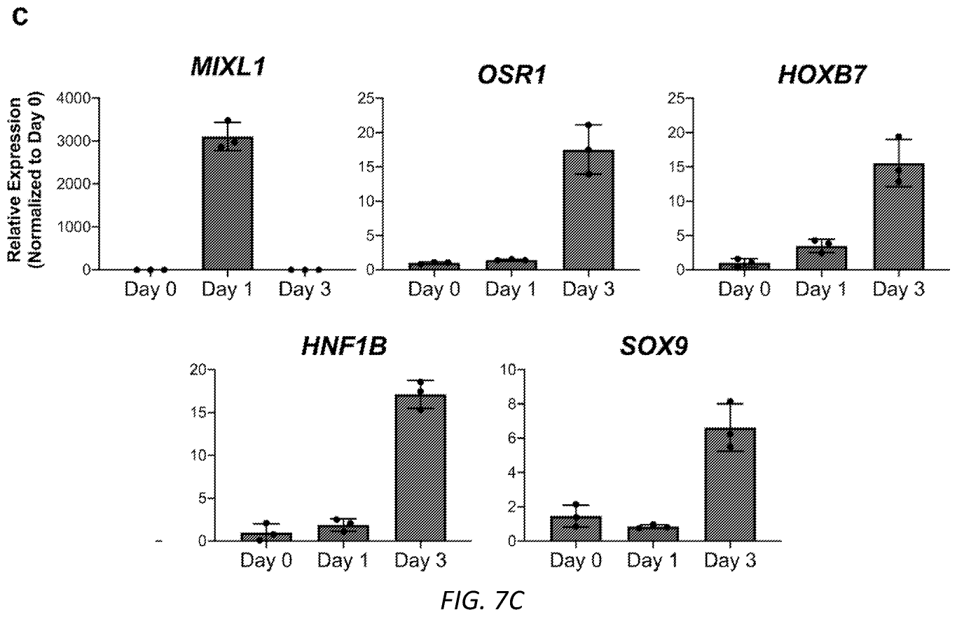

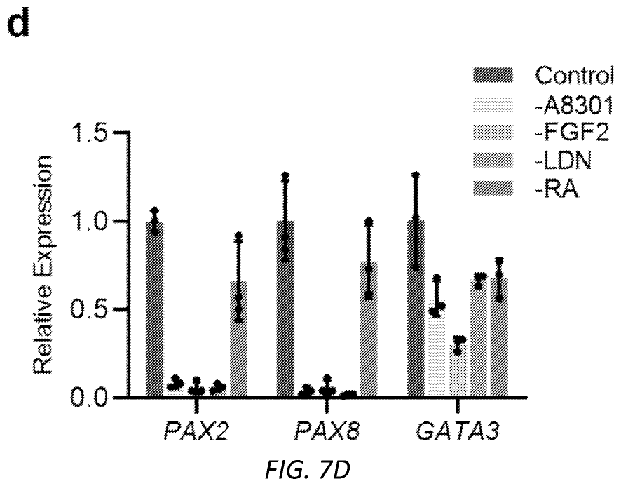

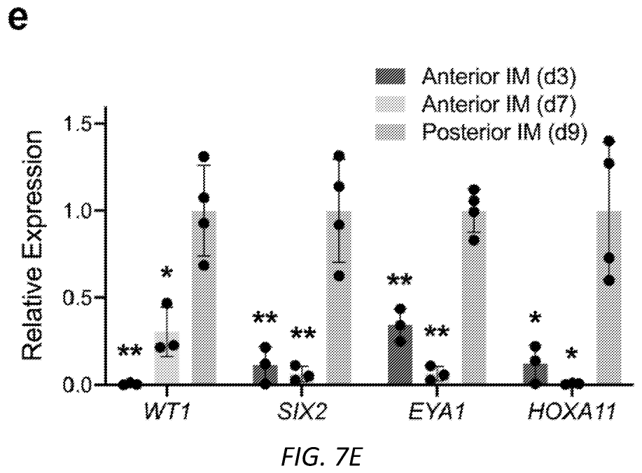

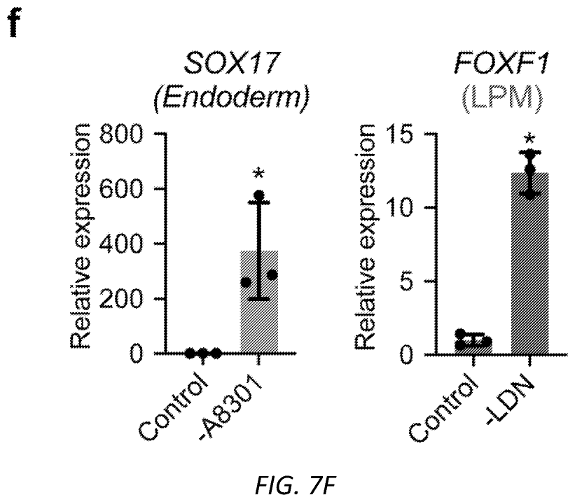

To establish a robust protocol for generation of UB structures (as summarized in FIG. 1a) we first sought to define conditions to efficiently differentiate hPSCs into pronephric IM. To facilitate these studies, we used CRISPR-Cas9 to introduce an in-frame mScarlet fluorescent reporter allele at the GATA3 locus (FIG. 6), a transcription factor expressed persistently from the earliest stages of pronephric development and maintained throughout the CD epithelium¹⁵. Since the pronephric IM derives from early-stage primitive streak¹⁶, we first used a combination of gastrula-stage signaling factors, including WNT (with the GSK3B inhibitor CHIR99021), FGF2, BMP4, and TGFβ (Activin A) as previously described to induce mesodermal fates from hPSCs¹⁷. This led to the rapid formation of TBXT⁺ mesendodermal progenitor cells with 96.6±2.1% efficiency (FIG. 1b and FIG. 7b) following a 30-hour exposure (FIG. 7a), with an abrupt decrease in expression of pluripotency genes such as NANOG (FIG. 1c).

From the primitive streak stage, which in vivo comprises multipotent progenitors, the cells were treated for 48 hours to optimize conditions for induction of IM fate. We found that a combination of retinoic acid (RA), FGF2, and inhibitors of BMP and TGFβ signaling enabled efficient specification of PAX2-positive IM progenitors. Consistent with an anterior or pronephric fate, on average 88.5±2.4% of cells at day 3 were positive for both GATA3 and PAX2 by immunofluorescent staining (FIG. 1d-e). We also confirmed similarly high levels of expression of other pronephric IM markers including PAX8, LHX1, and HOXB7 (FIG. 1d and FIG. 7c). Conversely, genes expressed in posterior IM regions, such as WT1, SIX2, EYA1 and HOXA11, were either absent or expressed at negligible levels in the cultures in contrast to their expression in differentiated metanephric progenitors⁵ (FIG. 7e). The cells rapidly lost expression of the mesendoderm progenitor genes TBXT and MIXL1 as they adopted an IM fate from days 1-3 (FIG. 1c and FIG. 7c). Through subtraction of individual components, we found that this particular signaling combination was highly synergistic and each factor was required for robust IM specification (FIG. 7d). FGF2 was particularly important, consistent with the established role for FGFs in the early stages of embryonic kidney development^{4, 18-21}. Inhibition of both BMP (with LDN193189) and TGFβ (with A83-01) signaling was required for the efficient repression of competing lateral plate mesoderm (FOXF1) and definitive endoderm (SOX17) fates, respectively (FIG. 7f). Taken together, these initial differentiation stages represent a straightforward and rapid method to generate pronephric progenitor cells with high efficiency.

Following its initial specification, the pronephric IM generates a cord of cells termed the nephric duct (ND; alternatively named the Wolffian duct), which migrates caudally toward the metanephric mesenchyme. To facilitate these complex morphogenetic events, we transitioned the day 3 monolayer via dissociation and re-aggregation into spheroids that varied in size between 50-200 μm (FIG. 1f and FIG. 8a). Based on the high efficiency of IM induction the cells were plated into aggregates in bulk without sorting or purification. Additionally, the cells could be plated into patterned microwell plates to generate large numbers of uniformly sized and shaped aggregates (FIG. 8c). These structures were maintained in a serum-free growth medium containing only RA, FGF9, and later GDNF (FIG. 8b; neither FGF2 nor FGF8 were able to support differentiation), and over several days they underwent spontaneous organization whereby GATA3-expressing cells sorted together into a sphere and spatially excluded a smaller GATA3-negative cluster (FIG. 1f). The GATA3-expressing population remained positive for PAX2, PAX8, HOXB7 and LHX1, and it acquired expression of the ND markers EMX2 and RET (FIG. 1g and FIG. 8d). The signaling molecules WNT9B and WNT11, which are expressed in the developing ND, were also up-regulated during this period (FIG. 8e).

To further define the cell types present in the spheroids at day 7, we performed single cell RNA sequencing (scRNA-seq) and analyzed 609 cells after filtering for quality and multiplets. As shown in the Uniform Manifold Approximation and Projection (UMAP) in FIG. 1h, this yielded four related cell clusters. Clusters 2 and 3 shared high levels of expression of ND markers, including PAX2, PAX8, GATA3, EMX2, and RET (FIG. 1h and FIG. 8f). In agreement with published reports⁶, these cells also expressed KIT and CXCR4 (FIG. 8f). Conversely, ND markers were largely absent in clusters 0 and 1, which corresponded to the GATA3-negative cells shown in FIG. 1f. Instead, the GATA3-negative expressed stromal lineage markers PDGFRA and COL1A1 (FIG. 1h and FIG. 8f-g) and also WT1, OSR1, TBX18, and ALDH1A2 (FIG. 8f-h), which are found in the stroma surrounding the ND during mouse development²². The scRNA-seq analysis did not identify the presence of off-target lineage differentiation (FIG. 8i), such as neurectoderm (PAX6), paraxial mesoderm (PAX3, TBX6), or lateral plate mesoderm (HANDI) cells.

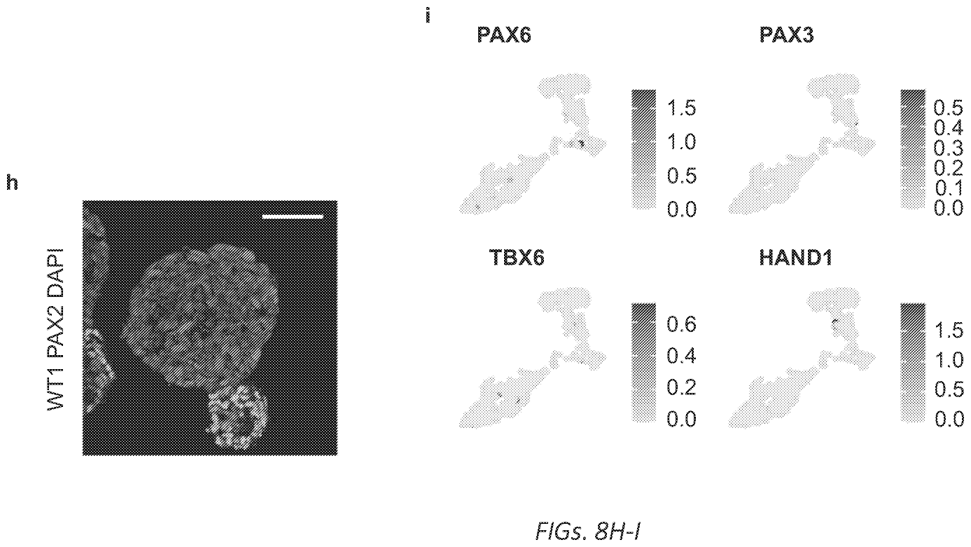

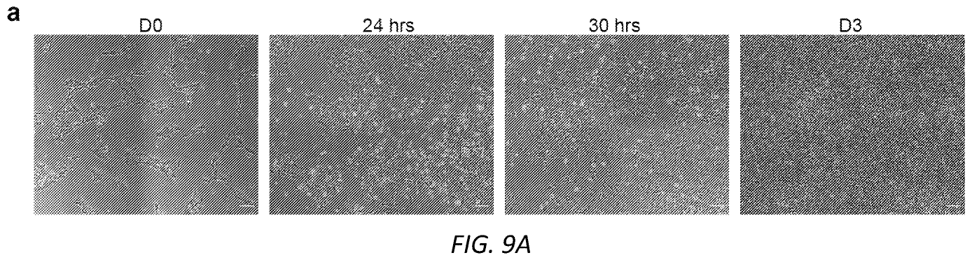

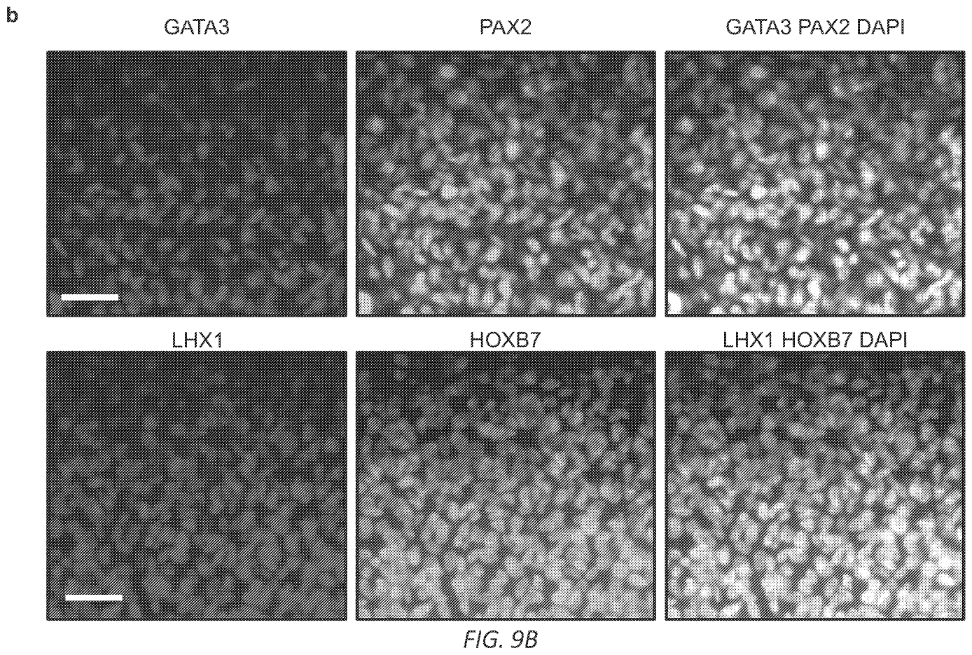

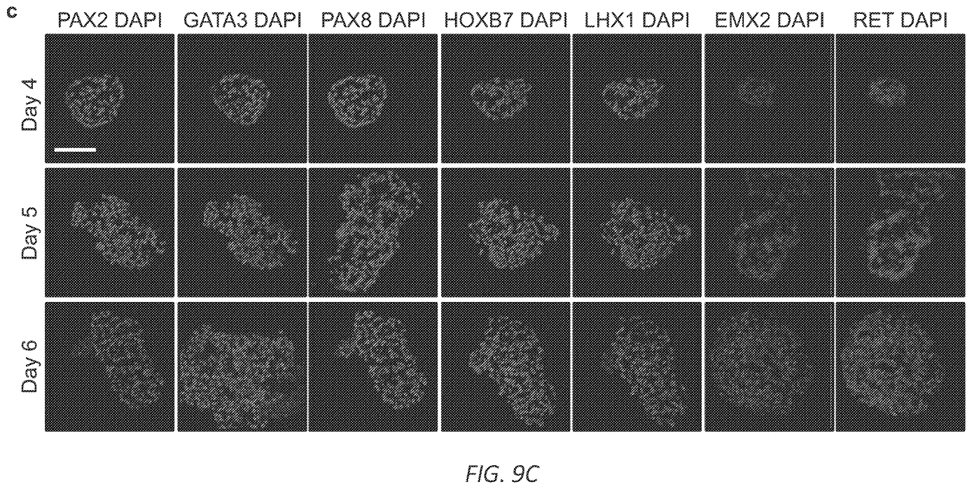

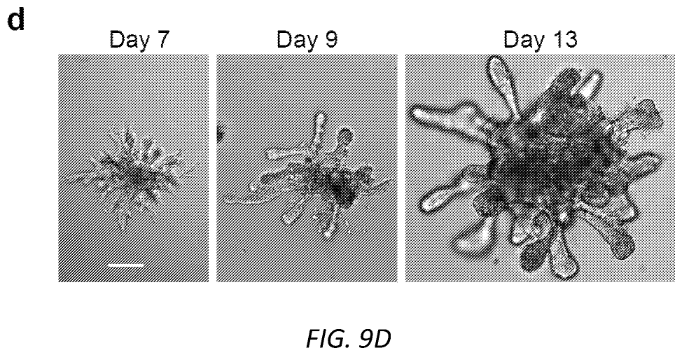

The analyses at day 7 revealed a ND identity, but CDH1 was only weakly and heterogeneously expressed in these cells (FIG. 1i). This signature was therefore analogous to the cells at the leading edge of the ND, a proliferative and chemotactic population that drives the caudal extension of the ND^{23, 24}. These ‘leader’ cells were recently characterized in vivo using single cell transcriptomics²⁵ (identified as ‘NDpr4’ cells), which identified that they express Aldh1a3 and are the likely immediate precursors to the UB. The differentiated spheroids at day 7 similarly expressed high levels of ALDH1A3 in the ND population (FIG. 1i), indicating that they exhibited a state similar to the NDpr4 population. Therefore, over 7 days of culture, hPSCs were efficiently differentiated into pronephric IM followed by three-dimensional spheroids comprising ND leader cells. Furthermore, this protocol could be applied across both hESC and hiPSC lines with consistent efficiency (FIGS. 9a-f).

### Example 2. Generation of Branching UB Organoids

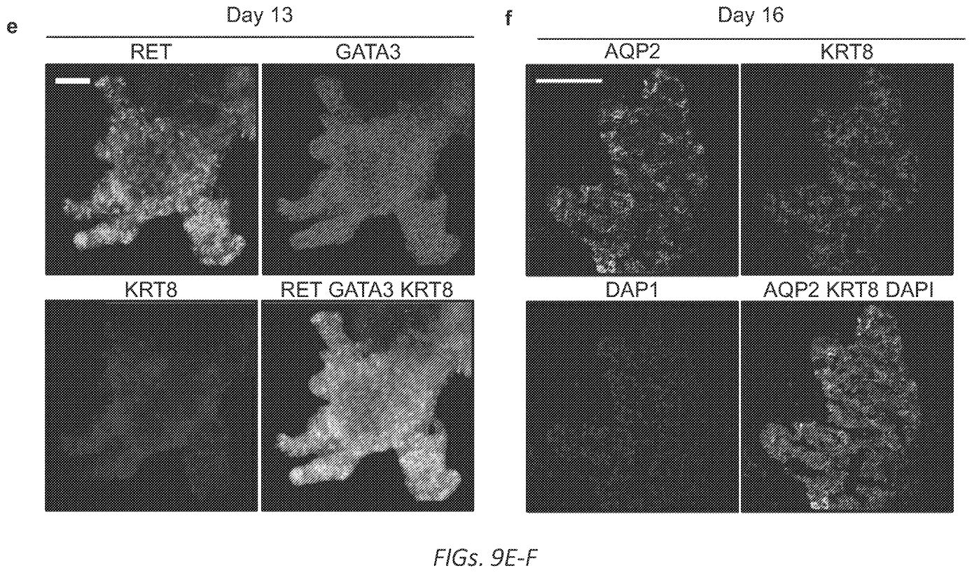

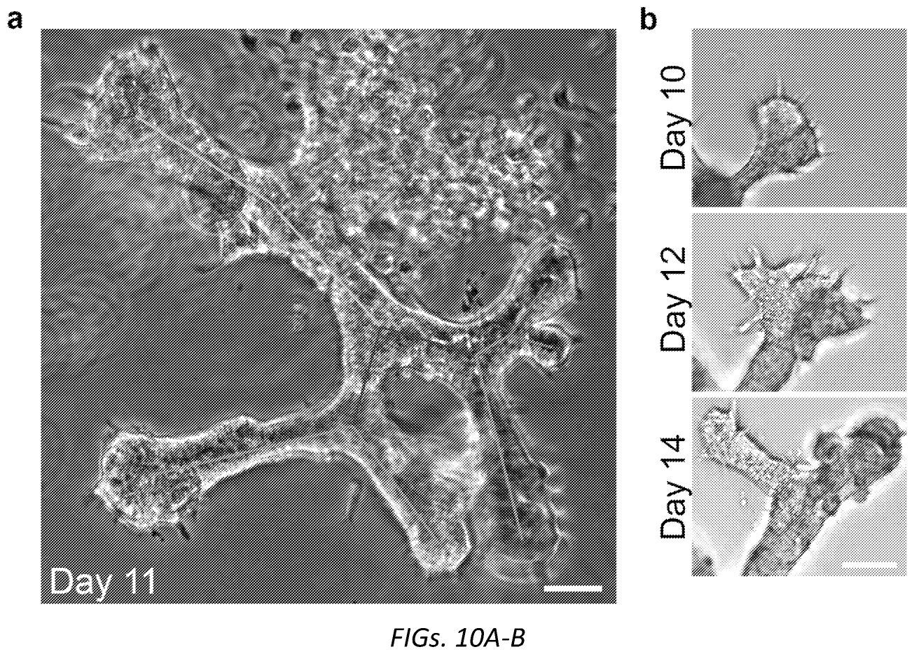

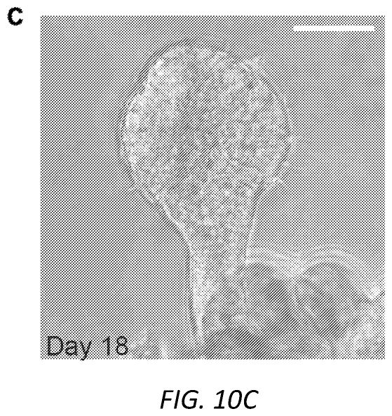

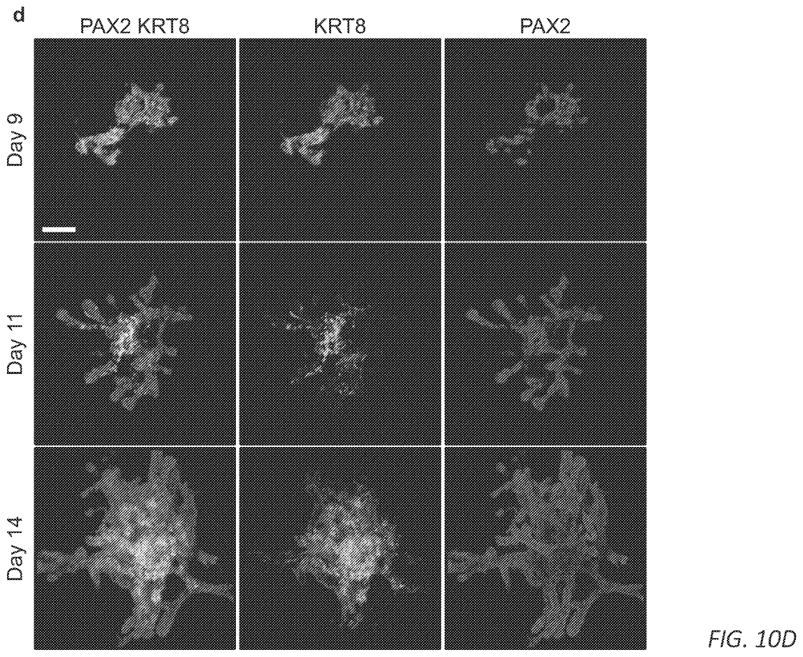

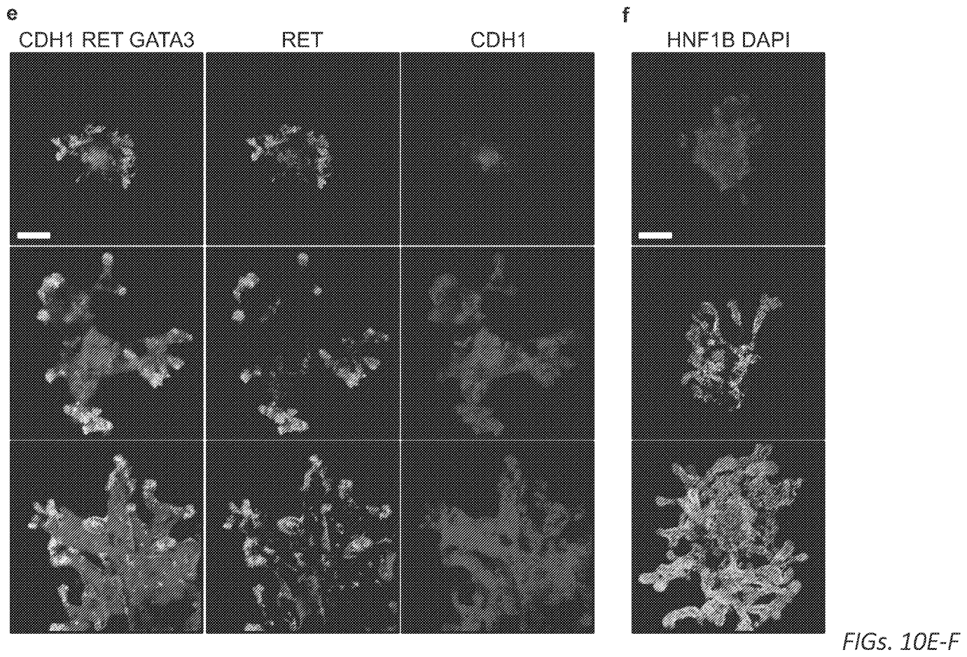

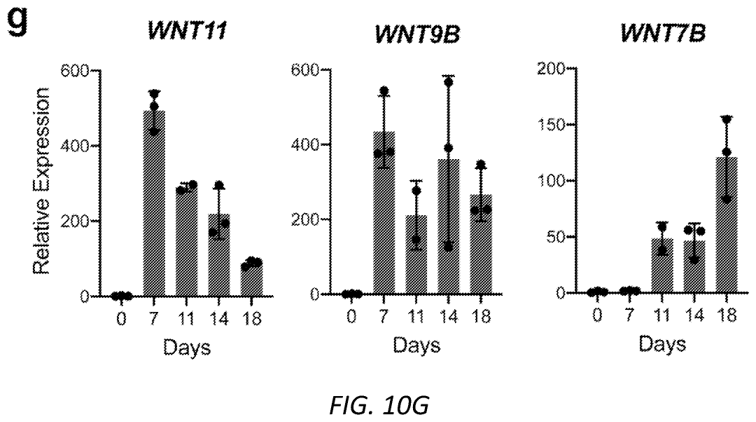

Branching morphogenesis and interaction with metanephric mesenchyme are defining features of the UB. Isolated fetal rodent UBs can grow as branching cultures in three-dimensional matrices²⁶, which was initially achieved with specialized conditioned media¹⁴ and later in more defined conditions²⁷. Drawing upon these published observations, we embedded the day 7 ND spheres into a dome of extracellular matrix (Matrigel) and exposed them to signaling conditions that promote a UB branching phenotype. We used serum-free conditions and a combination of growth factors including GDNF and FGF10, both of which have been well characterized for their ramogenic effects on the UB²⁸⁻³⁰, as well as inhibitors of BMP and TGFβ signaling given the known repressive effects of these pathway on UB budding and branching³¹⁻³³. These conditions led to a pattern of growth and morphogenesis over one week of culture that was unprecedented for hPSC-derived tissues in that they exhibited a rapidly growing and iterative branching pattern (FIG. 2a). Each of the original ND spheroids generated multiple buds that sprouted in all directions to form elongating tubules, and the epithelia then underwent branching by several rounds of terminal bifurcation (FIG. 2a-c and FIG. 10a-b). By day 14, the structures comprised an array of branched epithelial stalks with rounded tips that resembled the ampullae of the fetal UB (FIG. 2c). This pattern qualitatively paralleled UB branching behavior in vivo, and it very closely resembled the growth of fetal UBs in isolation in culture¹⁴. Beyond day 14, the rate of branching and overall growth in the UB organoids slowed and the bud tips at latter stages sometimes formed enlarged knob-like swellings (FIG. 10c).

The epithelium of the UB organoids maintained high expression of UB transcription factors PAX2 and GATA3, while expression of other developmental UB genes (including HNF1B, GRHL2, and CDH1) increased over time in a centrifugal pattern as the epithelialized phenotype was reinforced (FIG. 2c-d and FIG. 10d-f). Notably, spontaneous development of organized tip-stalk radial polarity was readily apparent as early as day 9, with KRT8 confined to the central or stalk-like regions and RET expression in the periphery of the epithelium (FIG. 2c-d). As the UB organoids grew, the relative proportion of tip domain cells decreased as demonstrated by waning WNT11 expression and an increase in the stalk marker WNT7B (FIG. 10g). Throughout these stages, however, RET remained expressed and specifically localized to only the distal tip domains (FIG. 2c-d), consistent with its pattern in the developing UB in vivo³⁴. Therefore the UB organoids closely mimicked normal stages of early development, at the levels of both morphogenesis and molecular patterning.

The morphogenesis of UB organoids was dependent upon on a balance of both activators and inhibitors of the MEK/ERK signaling pathway. The organoids exhibited severe reduction of growth and branching in the absence of exogenous GDNF (FIG. 2e), the most well-characterized growth factor in UB development³⁵. Conversely, removal of the MEK inhibitor U0126 from the culture medium led to highly branched structures that comprised very fine tubules with prominent cellular extensions and filopodia-like processes at the tips (FIG. 2e). This phenotype suggested that unopposed MEK activation prevented the ND leader cells from undergoing maturation and complete epithelialization. This effect on epithelial morphology paralleled observations previously described in the ND leader cells in the chick embryo²⁴.

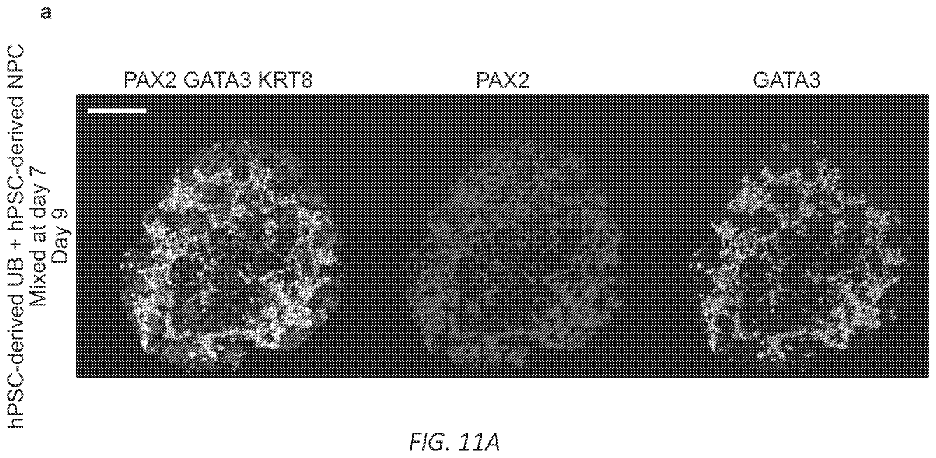

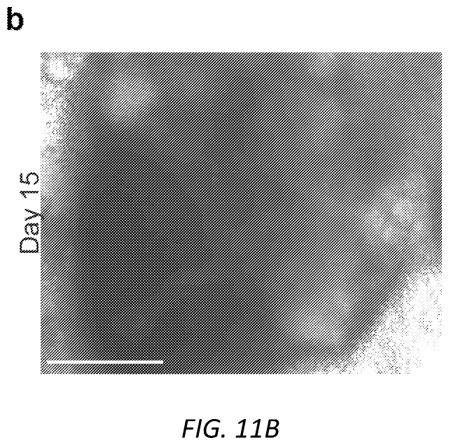

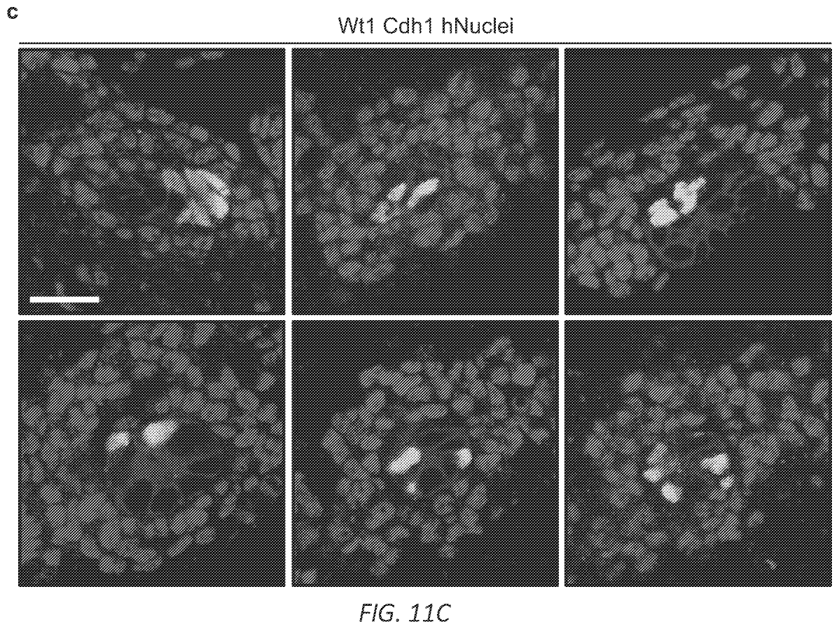

Next, we assessed the competence of UB organoids to participate in tissue-tissue interactions in the nephrogenic niche. UB cells at day 7 were combined with putative metanephric progenitors derived during kidney organoid differentiation⁵, and the resulting UB and metanephric tissues compartmentalized within the inner and outer parts of the organoids, respectively (FIG. 11a). We never observed meaningful branching or maintenance of undifferentiated nephron progenitors (FIG. 11b), similar to what has been reported elsewhere^{36,37}. Consequently, we used a well-established fetal kidney explant culture system³⁸ to specifically interrogate the function of the UB organoids. Murine metanephroi from embryonic day 12.5 were dissociated and mixed with a small proportion of hPSC-derived UB cells at a ratio of 10:1 (FIG. 2f). We cultured the explants for 72 hours and analyzed the nephrogenic zones for the presence of organoid-derived cells using a human-specific antibody. We frequently observed human cells incorporated in the UB tips surrounded by Six2⁺Wt1⁺ nephron progenitors (FIG. 2g-h and FIGS. 11c-d) in each of the explants (n=4) examined, and their close association with nephrogenic cells implied that they were responding appropriately to the murine patterning cues. Conversely, human cells were never observed in the capping mesenchyme itself as their fate was already restricted to the UB lineage.

### Example 3. Differentiation of Collecting Duct Epithelia in UB Organoids

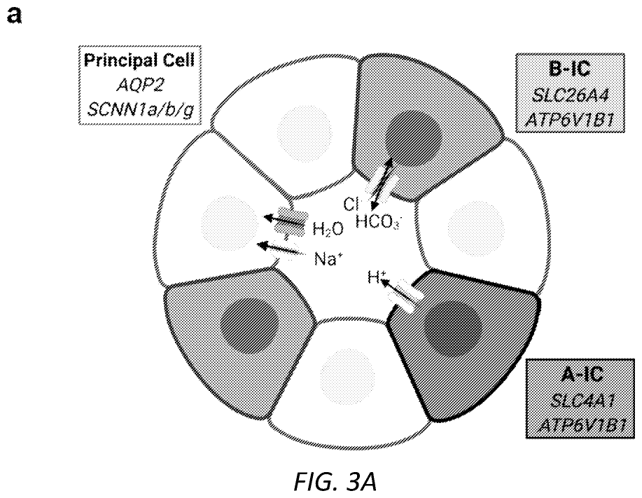

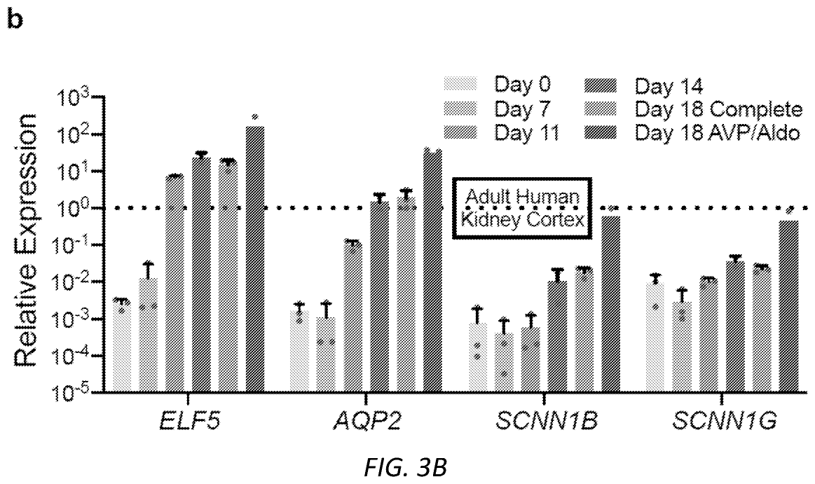

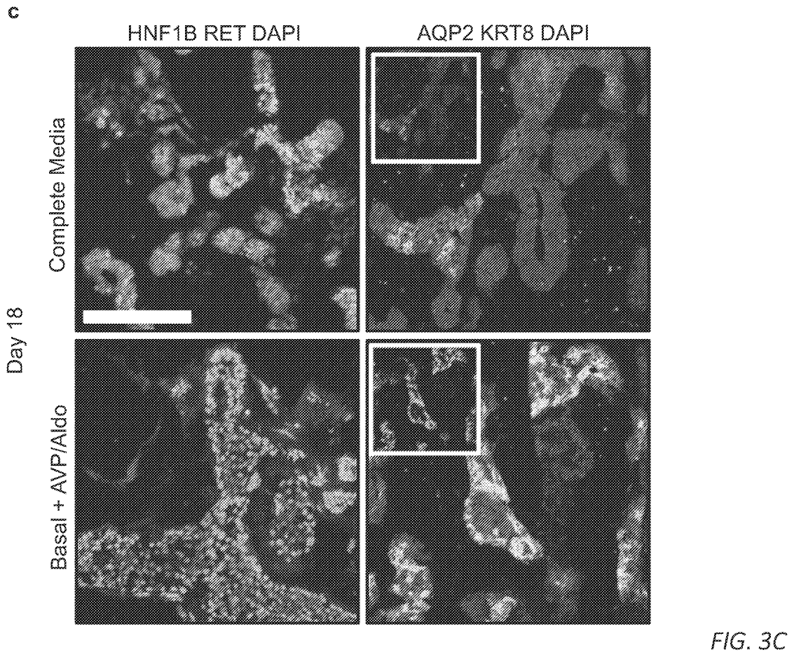

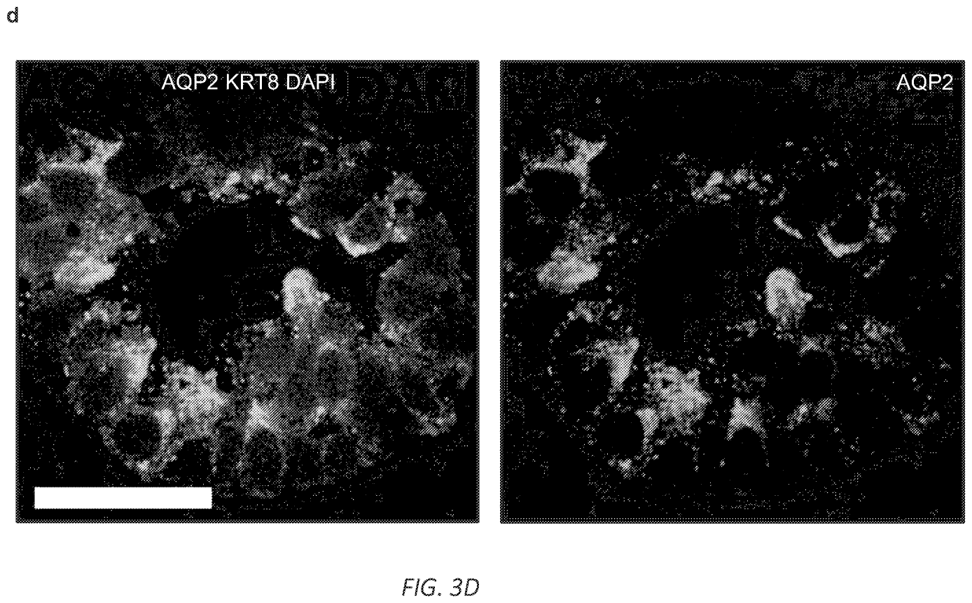

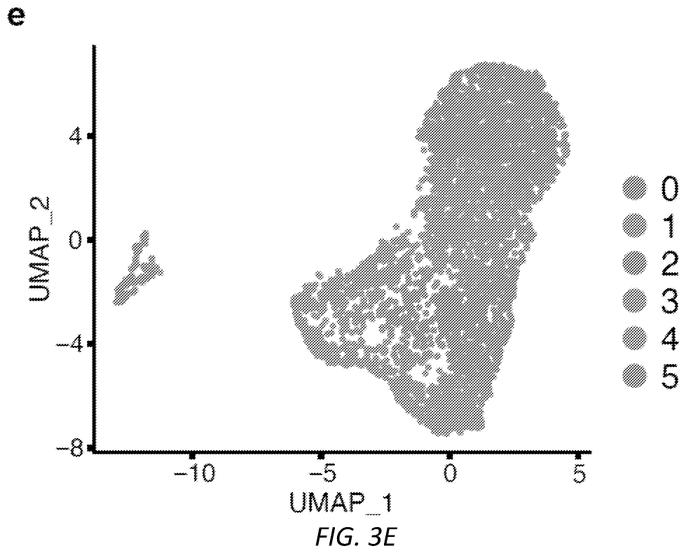

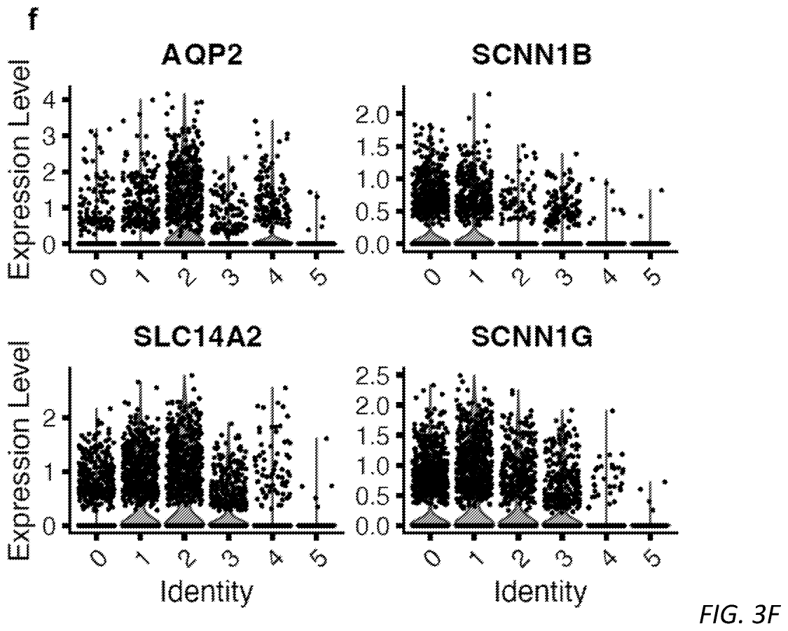

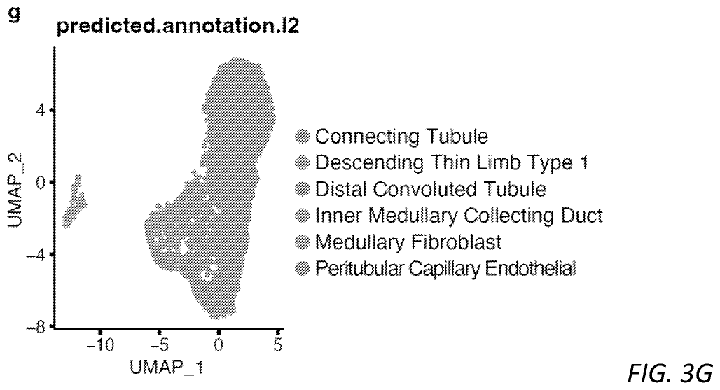

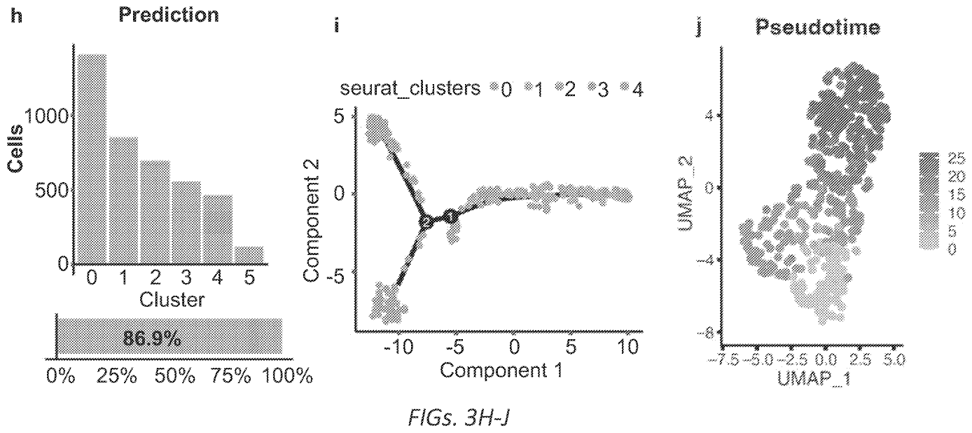

We monitored the developing UB organoids for the formation of differentiated CD cell types (schematized in FIG. 3a). They manifested a progressive increase in expression of principal cell markers at the transcriptional level between days 11-18, including the transcription factor ELF5 and water transporter AQP2 (FIG. 3b). As immunofluorescent staining revealed only few cells with low level AQP2-positivity (FIG. 3c), however, we hypothesized that the culture medium was inhibitory to epithelial maturation since it was designed to support the progenitor state. When we replaced this medium at day 14 with a basal medium devoid of other factors and supplemented only with the hormones arginine vasopressin (AVP) and aldosterone (Aldo), within 3-4 days there was repression of progenitor genes and significant up-regulation of ELF5 and AQP2 (FIG. 3b-c). The level of AQP2 exceeded expression in a sample of human kidney cortex by greater than 30-fold when assessed by qPCR, although PCs are only a minority of the many cell types in the kidney cortex. We therefore refer to these as CD organoids under these differentiation conditions, and they contained numerous AQP2+ cells throughout the tubular epithelia that was frequently localized to the luminal (apical) membrane (FIG. 3c-d), indicative of efficient PC specification and some extent of maturation.

To better characterize the differentiated cell types in the CD organoids, we generated a scRNA-seq dataset of 4,095 cells isolated at day 18. The cells segregated into six clusters as shown in FIG. 3e. Clusters 0-4 were tightly associated and constituted the vast majority (>97%) of cells, while only 118 cells were found in the distant cluster 5. Based on their high expression of representative marker genes including both transcription factors and functional channels/transporters (FIG. 3f), clusters 0-4 were identified as CD PCs. For an unbiased approach to further categorize these clusters, we used the recently described Azimuth toolkit³⁹ to perform reference mapping of our dataset to a multimodal dataset of 64,693 adult human kidney cells⁴⁰. Indeed, 86.9% of cells in our dataset mapped to a predicted ‘Inner Medullary Collecting Duct’ fate (FIG. 3g-h) during this analysis, in agreement with our conclusion that CD organoids comprised a large majority of PCs.

Aside from a CD fate, the next three highest predicted cell types in the Azimuth integration analysis were descending thin limb (dTL), distal convoluted tubule (DCT), and connecting tubule (CNT) at 7.6%, 4.6%, and 0.8% (FIG. 3g-h), respectively, which all derive from the metanephric lineage. This was surprising considering that expression of the canonical markers of metanephric nephron segments was absent in the dataset; for example, the highly specific DCT marker SLC12A3 was undetected. By reasoning that these segments were more likely to resemble the CD at early progenitor states due to overlapping expression of transcription factors, we hypothesized that the CD organoids contained cells that represented a continuum of differentiation. Lineage trajectory interrogation using Monocle on a downsampled subset of cells from clusters 0-4 showed a predicted lineage projection (FIG. 3i-j) that supported a model in which clusters 0 and 1 were the most differentiated while clusters 3 and 4 were most immature. Indeed, clusters 3 and 4 contained nearly all of the cells that mapped to non-CD identities using Azimuth (FIG. 3g), and these clusters had markedly lower prediction scores. Similarly, the prediction scores for all cells that mapped to non-CD fates were also quite low, suggesting that reference mapping to adult tissues may be less accurate when querying more immature or undifferentiated cell types.

### Example 4. Induced PCs Exhibit ENaC-Mediated Sodium Transport

The CD mediates hormone-responsive reabsorption of sodium and water via the epithelial sodium channel (ENaC) and AQP2 channels, respectively. In addition to AQP2, the PCs in the organoids at day 18 exhibited expression of ENAC subunits (SCNN1A/B/G) (FIG. 4a); in particular, the beta and gamma subunits were most abundant transcriptionally and we confirmed expression at the protein level using an antibody specific to SCNN1B, while the alpha subunit (SCNN1A) was expressed at lower levels. With their expression of ENAC and apicobasal polarity (FIG. 3d), we hypothesized that the cells might exhibit physiologic activity. The ENaC-mediated sodium reabsorption is an electrogenic process, so we transitioned organoid-derived cells to a two-dimensional transwell format to investigate their transepithelial electrophysiologic properties. Organoids were dissociated and re-plated in 2D tissue culture plates in a medium previously described for use with mouse CD cell lines¹¹. From this emerged a proliferative cell line that formed a confluent homogenous epithelium (FIG. 4b), which we were able to serially passage while maintaining the epithelial phenotype.

Following seeding, the cells developed a robust transepithelial resistance as they became confluent, which typically stabilized in the range of 5,000-6,000 Ohms·cm² by day 3-4 (FIG. 4c). A significant voltage (−60-80 mV) and calculated current (−10 to −15 μA·cm⁻²) also emerged over this period, both of which reached a peak 1-2 days after the resistance had plateaued (FIG. 4d-e). To test for the presence of ENaC-mediated sodium current we briefly exposed cells to amiloride, a diuretic that is a specific antagonist of ENaC. The addition of amiloride completely abolished the transepithelial voltage and current within several minutes while simultaneously raising the transepithelial resistance (FIG. 4e), essentially reducing the open-circuit current to zero and demonstrating the cells' exhibited robust ENaC-mediated sodium reabsorption. We further demonstrated the same response in a closed-circuit system using voltage clamping in an Ussing chamber, which allowed for real-time measurements of short-circuit current. In this system, amiloride at 10 UM inhibited >95% of the short-circuit current within several minutes, and further increasing the concentration to 100 μM subsequently eliminated the small amount of residual current (FIG. 4f). In fact, in the Ussing chamber the cells displayed a stereotypic pharmacokinetic dose-response relationship to amiloride with an estimated IC50 of 0.37 μM (FIG. 4g), which is within the expected range of 0.1-0.5 μM⁴¹.

In vivo, ENaC activity is positively regulated by mineralocorticoid signaling, so we tested whether the CD organoid-derived cells exhibited physiologic response to aldosterone. The cells were first cultured in the absence of dexamethasone for several days to wash out any glucocorticoid effect, and we then exposed the cells to varying concentrations of aldosterone for 24 hours. Aldosterone induced a dose-dependent increase in transepithelial voltage and current (FIG. 4h) and a statistically significant ~2.5-fold increase in amiloride-sensitive current at 10-100 nM (FIG. 4i). Therefore, the epithelium exhibited intact physiologic response to mineralocorticoid hormone signaling. Unlike the ENaC subunits, there was little expression of potassium channels in the organoid at day 18 with the exception of KCNJ16, which is found in the inner medullary CD⁴².

### Example 5. Induction of Intercalated Cells within CD Organoids

Although PCs differentiated readily under permissive culture conditions, scRNA-seq data revealed an absence of the IC-specific transcription factor FOXI1 and of its obligatory upstream regulator TFCP2L1⁴³. Because the organoids so strongly exhibited a signature consistent with inner medullary CD (FIG. 3g-h), we hypothesized that these IC-promoting factors might be regionally restricted along the corticomedullary axis of the CD. Using data generated from microdissected nephron segments from the adult mouse kidney⁴², we indeed found that expression of both Foxi1 and Tfcp2l1 were completely and specifically absent in the inner medullary CD. While markers of the transitional cells that exhibit both PC and IC characteristics⁴⁴, such as SEC23B, PARM1, and SYT7, were weakly but variably expressed within the organoids, the low level of TFCP2L1 likely made the cells refractory to spontaneously inducing FOXI1 and adopting the IC phenotype.

Based on prior reports that Foxi1 is necessary for IC development⁴⁵, we generated a transgenic hPSC line for temporally inducible expression to determine whether FOXI1 is sufficient for cell specification (FIG. 5a-b). Exposure of CD organoids to doxycycline to induce expression of FOXI1 from days 14-18 resulted in a heterogenous expression pattern that was analogous to the normal salt-and-pepper distribution of FOXI1 in vivo (FIG. 5c). We also observed a similar pattern of cells with strong expression of the V-type ATPase subunit ATP6VIB1 distributed within the epithelium (FIG. 5c-d), confirming that FOXI1 was sufficient to induce IC specification. Although AQP2⁺ PCs still formed in the presence of doxycycline, these cells were distinct from those that expressed IC markers. We detected abundant expression of the B-type IC marker SLC26A4 (FIG. 5e), the apical transporter that mediates tubular secretion of bicarbonate in the CD, while the A-type IC marker SLC4A1 was not observed.

We examined whether FOXI1 expression disrupted the electrophysiologic PC phenotype in the organoid-derived cell line. Transgene induction led to a statistically significant reduction in the transepithelial resistance to approximately 25% of control levels (FIG. 5f), consistent with a disruption of the epithelial tight junctions that are characteristic of PCs. More strikingly, it consistently reversed the direction of the transepithelial voltage (FIG. 5g), and the calculated open-circuit current further exemplified this stark contrast in the baseline electrical state between these two conditions (FIG. 5h). The induced cells also largely lost sensitivity to amiloride (FIG. 5h), so we hypothesized that FOXI1 led to the emergence of activity of the IC-specific V-type ATPase proton pump, which could have electrogenic function by mediating the movement of an unpaired proton (H⁺) across the apical membrane into the lumen. Indeed, the addition of the V-type ATPase inhibitor bafilomycin completely suppressed both the voltage and calculated current observed in the FOXI1-induced cells (FIG. 5g-h). Therefore, the inducible expression of FOXI1 was sufficient to toggle between PC and IC states that exhibit ENaC (amiloride-sensitive) and V-type ATPase (bafilomycin-sensitive) activities, respectively (FIG. 5i). We confirmed these results under closed circuit conditions in Ussing chambers, where FOXI1 induction led to reversal of the direction of baseline short-circuit current, a small increase in current following treatment with amiloride, and near complete inhibition of the current in response to bafilomycin (FIG. 5j).

ICs in the CD use V-type ATPase to generate large proton gradients and can acidify the urine to a pH of 5 in order to excrete dietary acid loads and maintain acid-base homeostasis. FOXI1-induced electrogenic activity of V-type ATPase coincided with increased acidification of the medium in the upper but not lower chamber of the transwell (FIG. 5k-l) and generation of a gradient across the epithelium of up to 0.5 pH units (FIG. 5m). Compared with control cells, the lower chamber was actually alkalinized to a modest but statistically significant degree. The changes occurred rapidly, in as little as 12 hours after replacement with fresh medium. This acidification phenotype was more consistent with that of A-ICs with the apical proton secretion. Therefore, FOXI1 was sufficient to promote an IC phenotype in the hPSC-derived CD, and it is possible that the specific IC phenotype was dependent on the culture environment.

### Example 6. Detailed Protocol

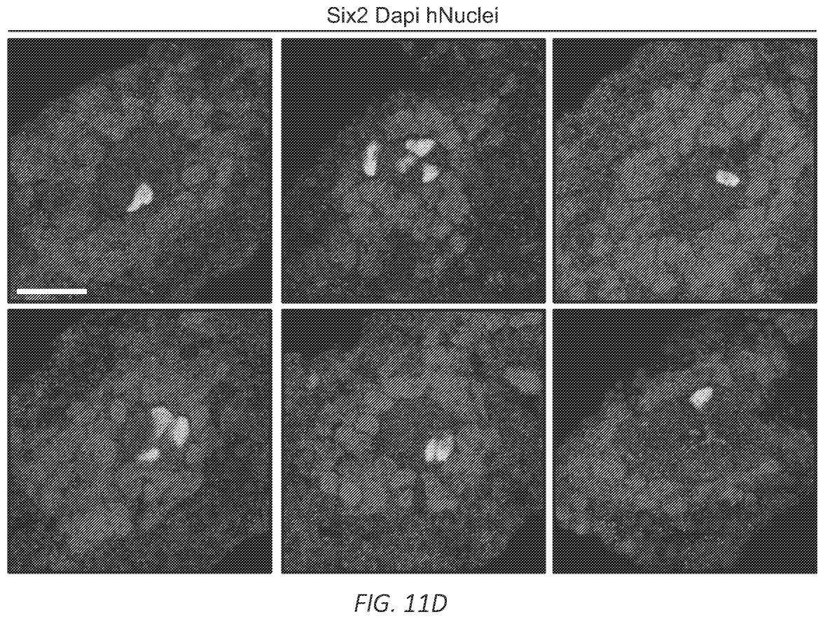

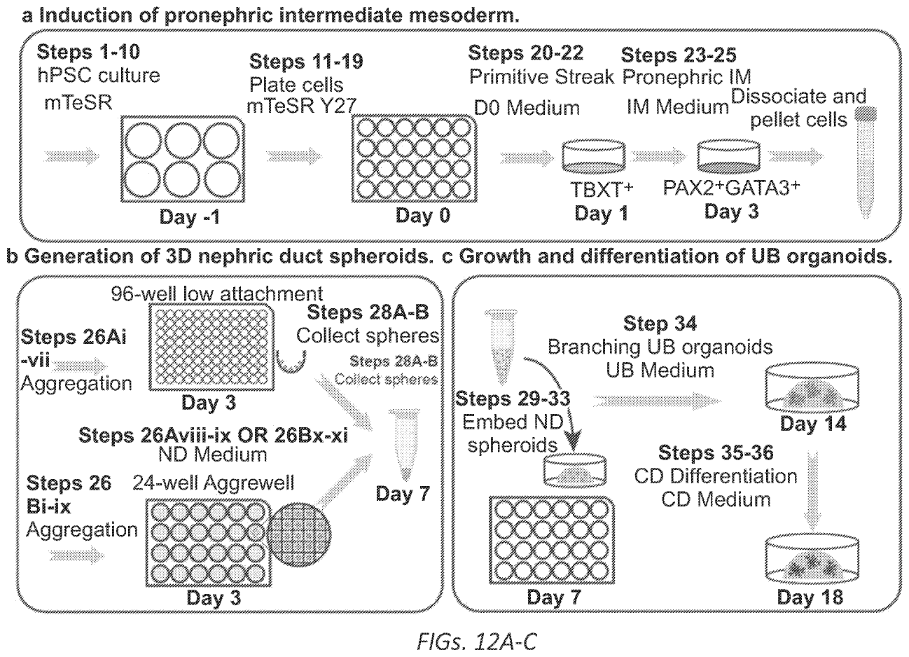

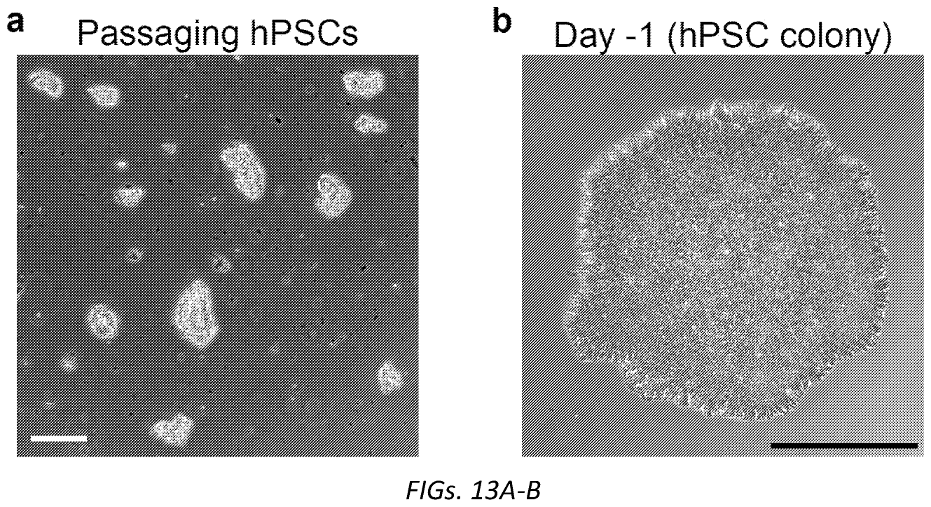

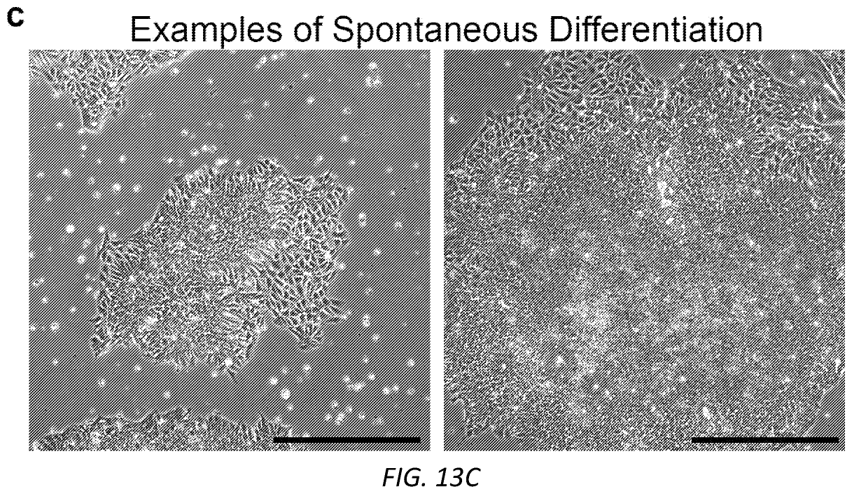

Feeder-free hPSC culture in mTeSR1 medium (Steps 1-10, FIG. 12A). We maintained hPSCs in six-well plates coated with LDEV-free hESC-qualified Matrix with mTeSR1 medium following routine culture recommendations from STEMCELL Technologies. Daily medium changes were performed, and hPSCs were passaged every 4-5 days as cell aggregates using Gentle Cell Dissociation Reagent with manual scraping followed by trituration. Typical colony morphology prior to passaging or plating cells is shown in FIG. 13a. hPSCs maintained using different conditions (e.g., other medium, basement membrane matrices, and/or passaging reagents) may be efficiently induced into UB organoids.

Plating hPSCs for differentiation (Steps 11-19, FIG. 12A). The first stages of differentiation were performed in monolayer cultures in 24-well plates (as shown in FIG. 12a). We plated single cells for differentiation when the colonies would otherwise be ready for passaging. Plating density was crucial in determining differentiation efficiency, with approximately 3.0×10⁴ cells/well in 24-well plates providing best results, although different lines may require individual optimization. In our experience ~85% confluent hPSCs could be split into ~30-40 wells of 24-well plates. In brief, hPSCs were dissociated with Accutase for 7 minutes, resuspended in mTeSR1 media supplemented with a ROCK inhibitor (10 μM Y27632), and plated into 24-well plates coated with hESC-qualified Matrigel Matrix on day-1. Approximately 24 hours after plating, the cells reached ~20-30% confluence and were ready to begin differentiation (FIG. 13b).

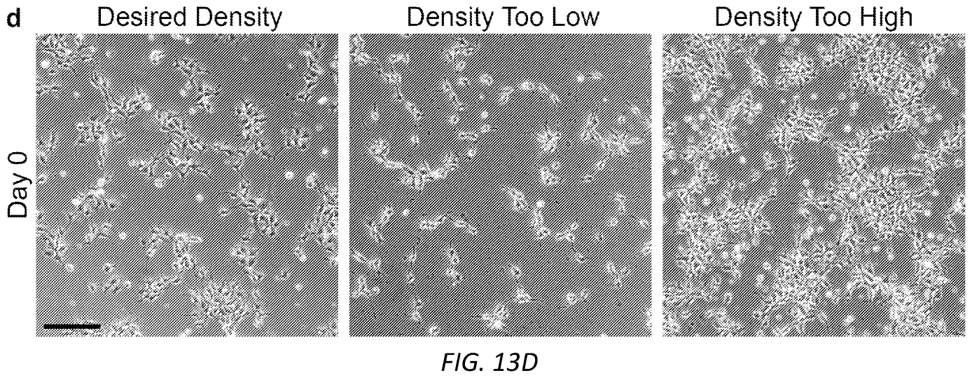

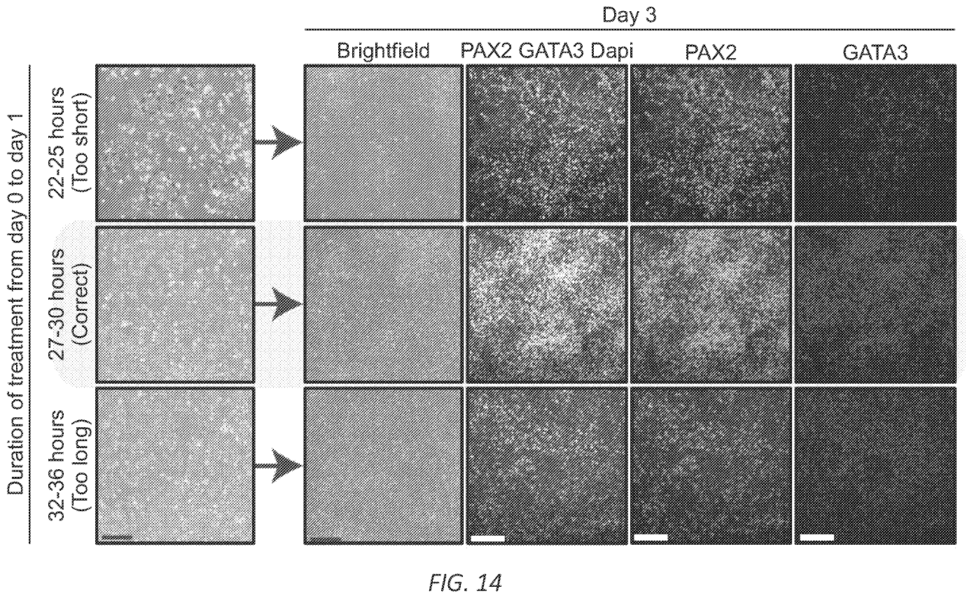

Specification of pronephric IM progenitor cells (Steps 20-25, FIG. 12A). Pluripotent cells were induced into primitive streak mesendodermal progenitors by addition of Day 0 (DO) Medium (Table A) that contained GSK3 Inhibitor/WNT activator CHIR99021 (5 μM), FGF2 (25 ng/ml), BMP4 (25 ng/ml), and Activin A (50 ng/ml) from day 0 to day 1. The rapid and efficient formation of these progenitors is essential for the downstream success of the protocol, and we found that the timing of this initial exposure was critical (as shown in FIG. 14). We relied on observing the morphological changes that occur during this process, as cells in tightly packed colonies eventually dispersed into single mesenchymal cells. After only 24 hours, there were most often colonies remaining that were not completely dispersed and the transition to a mesenchymal phenotype was incomplete. Typically at sometime between 28-32 hours, the remaining colonies had more completely dispersed and the cells were ready to move to the next step of the protocol. These visual morphologic cues were used to guide the timing of differentiation, especially when initially optimizing the methods. Further extending this stage to >34 hours also led to inadequate differentiation efficiency at later stages, as shown with staining for PAX2 and GATA3 in FIG. 14.

Once the cells had reached a dispersed mesenchymal appearance on day 1, the medium was changed to IM Medium (Table B) that contains FGF2 (25 ng/ml), LDN193189 (0.1 μM), A83-01 (1 μM) and RA (0.1 μM). Fresh IM medium was again added on day 2. During the period from days 1-3, the cells proliferated rapidly to become confluent and fairly dense. On day 3 of differentiation, the cells reached the pronephric IM stage and exhibited expression of the critical markers GATA3 and PAX2 (FIG. 14).

TABLE A
D0 Medium

| | | | Stock | | Final | |
|---|---|---|---|---|---|---|
| | | | concen- | | concen- | |
| Components | Vendor | Cat. no. | tration | | tration | |
| Advanced RPMI | Thermo | 12633-012 | — | | 99% | |
| 1640 medium | Scientific | | | | (vol/vol) | |
| GlutaMAX | Thermo | 35050061 | — | | 1% | |
| | Scientific | | | | (vol/vol) | |
| CHIR99021 | Cayman | 13122 | 10 | mM | 5 | μM |
| | Chemical | | | | | |
| FGF2 | PeproTech | 100-18B | 50 | μg/ml | 25 | ng/ml |
| BMP-4 | PeproTech | 120-05ET | 50 | μg/ml | 25 | ng/ml |
| Activin A | PeproTech | 120-14E | 50 | μg/ml | 50 | ng/ml |

TABLE B
IM Medium

| | | | Stock | | Final | |
|---|---|---|---|---|---|---|
| | | | concen- | | concen- | |
| Components | Vendor | Cat. no. | tration | | tration | |
| Advanced | Thermo | 12633-012 | — | | 99% | |
| RPMI 1640 | Scientific | | | | (vol/vol) | |
| medium | | | | | | |
| GlutaMAX | Thermo | 35050061 | — | | 1% | |
| | Scientific | | | | (vol/vol) | |
| FGF2 | PeproTech | 100-18B | 50 | μg/ml | 25 | ng/ml |
| A83-01 | Cayman | 9001799 | 10 | mM | 1 | μM |
| | Chemical | | | | | |
| LDN-193189 | Cayman | 19396 | 1 | mM | 0.1 | μM |
| | Chemical | | | | | |
| RA | Sigma Aldrich | R2625 | 1 | mM | 0.1 | μM |

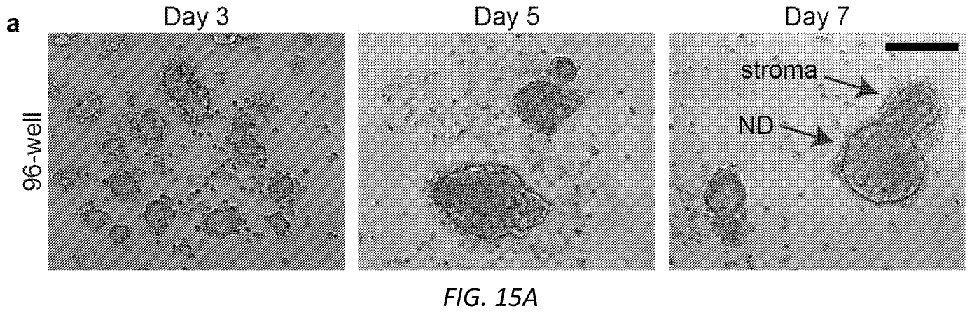

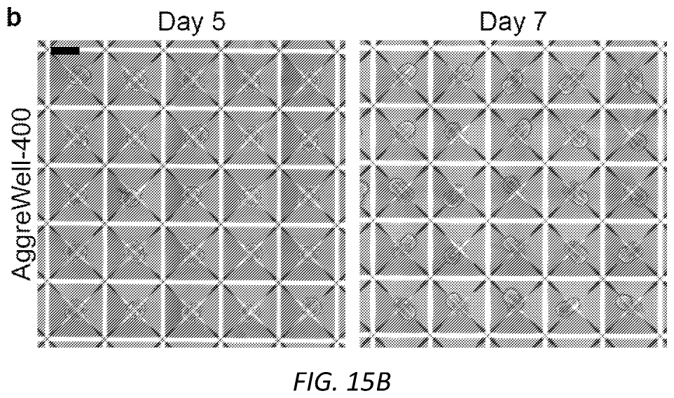

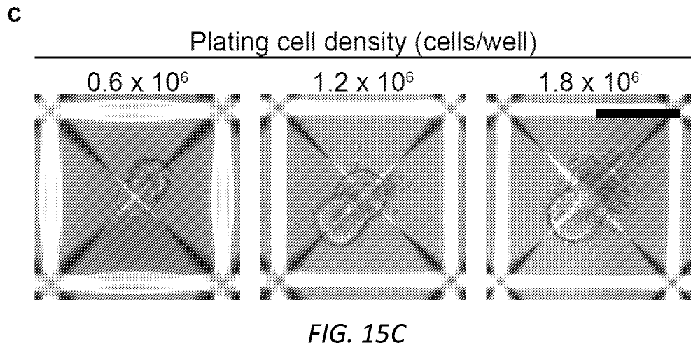

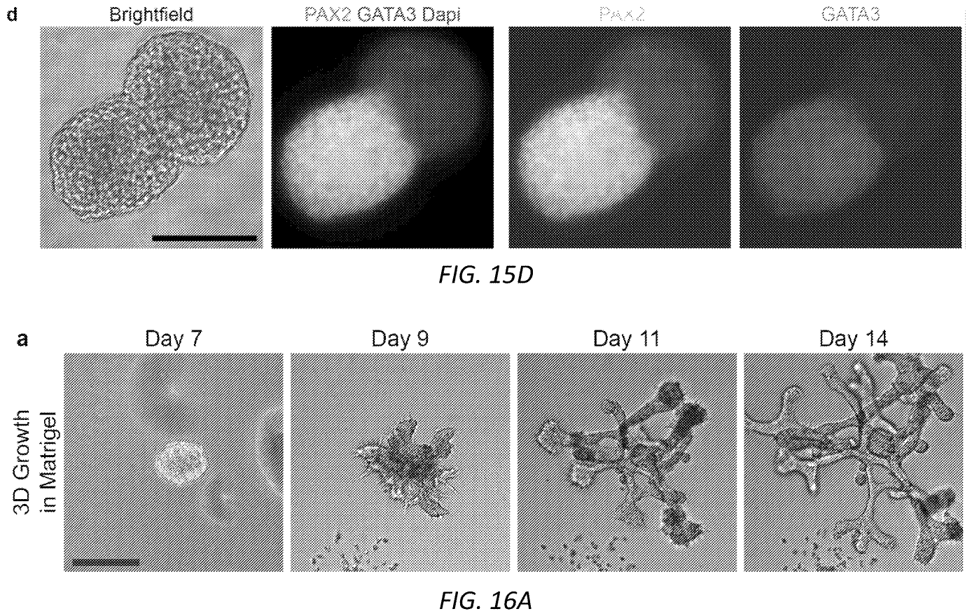

Formation of 3D nephric duct spheroids (Steps 26A-B, FIG. 12B). On day 3, the cells (in the pronephric IM stage that exhibited expression of GATA3 and PAX2) from monolayer culture were transitioned to 3D spheroids via dissociation and re-aggregation. We delineated two techniques for aggregation (as schematized in FIG. 12b), detailed in the steps below. In the first method dissociated cells were plated at low density into 96-well round bottom, ultra-low attachment plates, followed by gentle centrifugation to form numerous small aggregates per well (FIG. 15a). Note that the relatively low cell density and low centrifugation speed were best, to prevent the formation of a single, large sphere in each well. The alternative strategy used patterned microwell (AggreWell-400; STEMCELL Technologies) plates, which can efficiently form large numbers (~1,200 per well in 24-well format) of uniformly-sized spheroids (FIG. 15b). The preparation of cell suspensions for either method was similar. In brief, monolayer cells were dissociated into single cells with Accutase, and then pelleted by centrifugation. The cell pellet was then resuspended in ND Medium (Table C), which contained FGF9 (50 ng/ml) and RA (0.1 μM). For 96-well plates, cells were plated at a density of about 2.0-3.0×10⁴ cells/well in 200 μl medium. For AggreWell-400 plates, the cells were plated at a density of about 1.2×10⁶ cells/well in 2 ml medium (~1,000 cells/microwell). We have also found that the cell density in the AggreWell could be decreased or increased (by at least 2-fold in either direction) to change the size of the spheroids without impacting the differentiation efficiency or general morphology (FIG. 15c). At day 5, a half-volume medium change was performed to introduce GDNF (50 ng/ml) without disturbing the small spheroids. All handling, manipulation, or medium changes in these plates was performed gently and with minimal agitation to prevent the clumping of individual spheroids into large aggregates. By day 7 (and typically observable by days 5-6), the structures matured into ND spheroids that were compartmentalized into a dominant ND domain (that expresses PAX2, GATA3, and RET) and a smaller stromal domain that did not express ND markers¹⁸ (FIG. 15d).

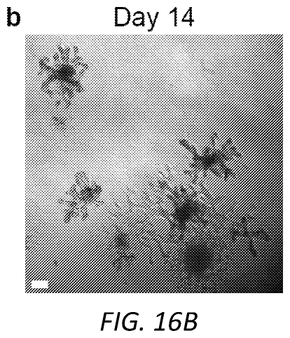

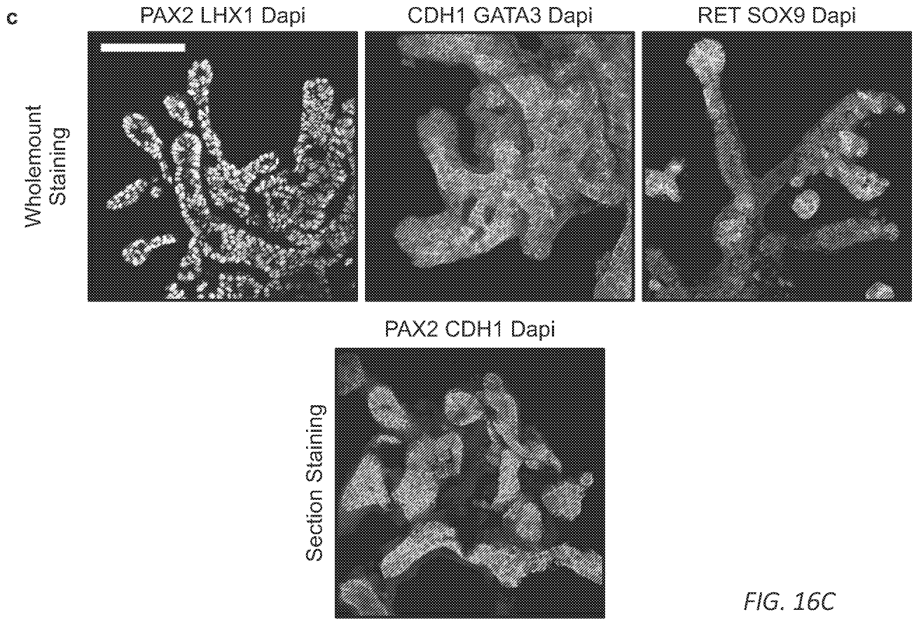

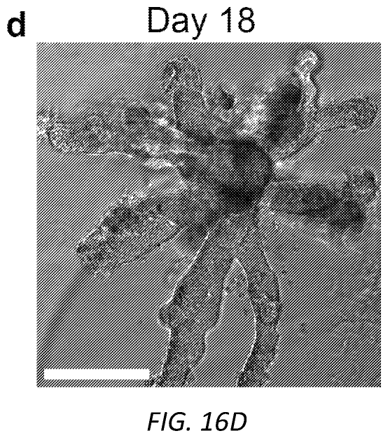

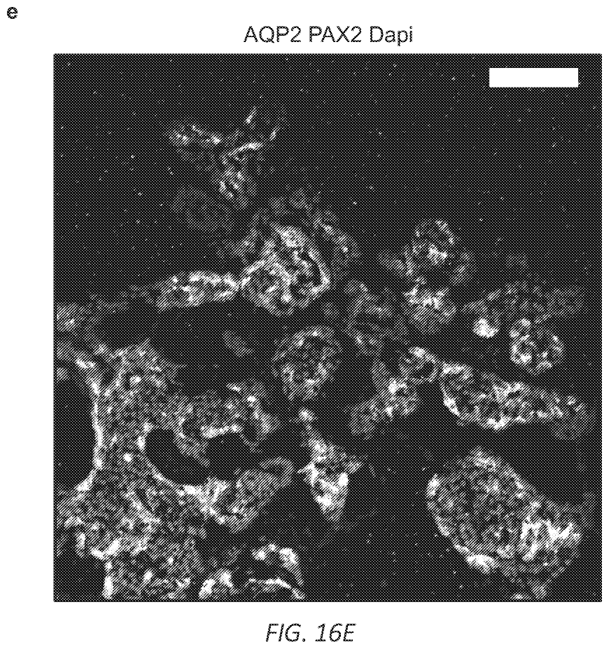

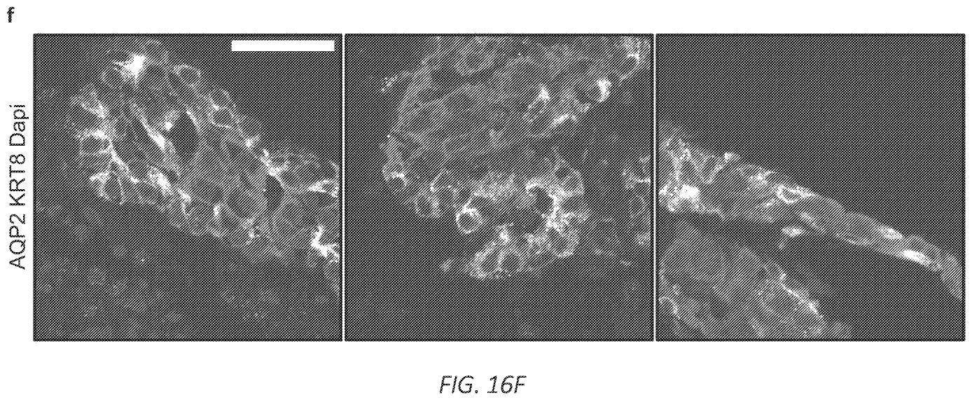

The physical segregation of ND and stromal lineages was readily apparent in a successful differentiation. We have not observed any evidence that the stromal cell domain is required for the subsequent growth and morphogenesis of the organoids. Once the spheroids were embedded in Matrigel, these cells dispersed through the matrix and did not maintain any close contact with the UB epithelium. For example, loose stromal cells are shown in the bottom of the images in FIG. 16a. Therefore, it was not necessary to maintain the ND-stroma association during the embedding process (below), as they frequently separated during pipetting. Conversely, the presence of these cells did not inhibit organoid growth, so they do not need to be intentionally removed.

TABLE C
ND Medium

| | | | | |
|---|---|---|---|---|
| | | | Stock | Final |
| | | | concen- | concen- |
| Components | Vendor | Cat. no. | tration | tration |

Days 3-5

| Advanced RPMI | Thermo | 12633-012 | — | | 99% | |
|---|---|---|---|---|---|---|
| 1640 medium | Scientific | | | | (vol/vol) | |
| GlutaMAX | Thermo | 35050061 | — | | 1% | |
| | Scientific | | | | (vol/vol) | |
| FGF9 | R&D Systems | 273-F9- | 50 | μg/ml | 50 | ng/ml |
| | | 025/CF | | | | |
| RA | Sigma Aldrich | R2625 | 1 | mM | 0.1 | μM |

Days 5-7

| Advanced RPMI | Thermo | 12633-012 | — | | 99% | |
|---|---|---|---|---|---|---|
| 1640 medium | Scientific | | | | (vol/vol) | |
| GlutaMAX | Thermo | 35050061 | — | | 1% | |
| | Scientific | | | | (vol/vol) | |
| GDNF | PeproTech | 450-10 | 50 | μg/ml | 50 | ng/ml |
| RA | Sigma Aldrich | R2625 | 1 | mM | 0.1 | μM |

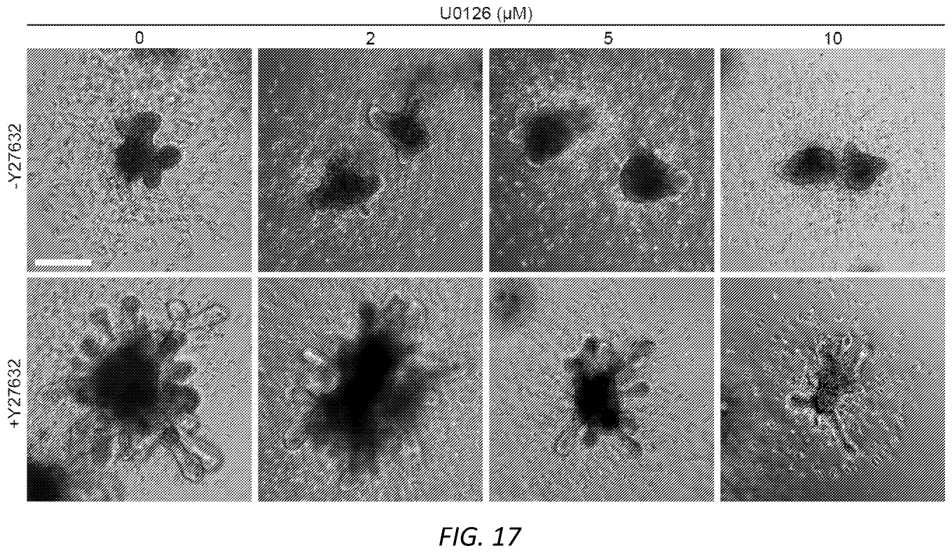

*Concentrations of U0126 and Y-27632 should be optimized for each pluripotent stem cell line used in the protocol, as shown in FIG. 17.

Generation of 3D UB and CD organoids (Steps 27-36, FIGS. 12B-C). On day 7, the ND spheroids were embedded into Matrigel Matrix for 3D growth as has been previously described for organoids comprising gastrointestinal tissues³³⁻³⁵. The spheroids were collected from 96-well or AggreWell plates, placed into Eppendorf tubes, and allowed to settle to the bottom by gravity. The supernatant was removed and the spheroids were re-suspended in cold 100% Matrigel Matrix. The spheroids/Matrigel were then plated into the center of 24-well plates as 45 μl droplets that maintain a domed 3D shape. The 24-well plate was incubated in 37° C. CO₂ incubator for ~60 minutes to allow the Matrigel to gel and solidify. At that point, UB Medium (Table D), which contains FGF10 (50 ng/ml), GDNF (50 ng/ml), CHIR (2 μM), LDN193189 (0.1 μM), A83-01 (1 μM), RA (0.1 μM) and U0126 (5 μM), was gently overlayed into the wells. The UB Medium was changed and refreshed every 3-4 days. During 7 days of in-gel culture, the spheroids sprouted stalks and underwent epithelialization. The resulting organoids exhibited branching morphogenesis, typically by terminal bifurcation (FIG. 16a). The structures express multiple markers of the developing UB including tip-localized expression of RET (FIG. 16b). Beyond day 14, the branching behavior of the organoids slows and they grow predominantly through stalk elongation.

We observed some degree of variability in the branching characteristics of UB organoids derived from different cell lines, which we hypothesize reflects the timing by which the ND cells epithelialize into a UB phenotype. The cell lines used included WA09 (RRID: CVCL_9773); iPSC72-3 (RRID: CVCL_A1BW): iPSC72-3-GFP (RRID: CVCL_C7HE); and BJFF.6 (RRID: CVCL_VU02), but other cell lines can also be used. For testing new cell lines, a troubleshooting experiment was performed to optimize growth and branching, with the major variables being the concentration of MEK inhibitor U0126 (between 0-10 UM) and the presence or absence of the ROCK inhibitor Y-27632 (0 or 10 μM). For H9 hESCs, we found that 5 μM U0126 is ideal and Y-27532 is not required¹⁸. Alternatively, with the cell line iPSC72-3³⁶, Y-27632 was required and U0126 significantly slowed branching (FIG. 17). As shown in FIG. 17, the desired morphologic phenotype was easily identifiable by day 10-11 of differentiation. Once these conditions were established for a particular cell line, we found they will work with high reproducibility without the need to repeat testing or optimization. The biological reason for the differential requirement for U0126 and/or Y-27632 among cell lines is unclear at this point in time.

Following the branching phase of growth, the organoids can be differentiated into CD epithelia by removal of the UB Medium. We have found that medium containing only arginine vasopressin (AVP, 10 nM) and aldosterone (Aldo, 10 nM) (CN Medium, Table E) was sufficient to promote spontaneous specification of principal cell-like fates with expression of AQP2 throughout the organoid (FIG. 16e-f). We have most frequently transitioned organoids to the CD Medium at day 14, but also found they are similarly responsive at any time between days 13-18, and principal cell markers are identified within 3-4 additional days of culture. During this period, the elongation of UB stalks ceases and the epithelia tend to modestly grow by increasing in caliber (FIG. 16d).

TABLE D
UB Medium

| | | | Stock | | Final | |
|---|---|---|---|---|---|---|
| | | | concen- | | concen- | |
| Components | Vendor | Cat. no. | tration | | tration | |
| Advanced | Thermo | 12633-012 | — | | 99% | |
| RPMI 1640 | Scientific | | | | (vol/vol) | |
| medium | | | | | | |
| GlutaMAX | Thermo | 35050061 | — | | 1% | |
| | Scientific | | | | (vol/vol) | |
| CHIR99021 | Cayman | 13122 | 10 | mM | 2 | μM |
| | Chemical | | | | | |
| GDNF | PeproTech | 450-10 | 50 | μg/ml | 50 | ng/ml |
| FGF10 | PeproTech | 100-26 | 50 | μg/ml | 50 | ng/ml |
| A83-01 | Cayman | 9001799 | 10 | mM | 1 | μM |
| | Chemical | | | | | |
| LDN-193189 | Cayman | 19396 | 1 | mM | 0.1 | μM |
| | Chemical | | | | | |
| RA | Sigma Aldrich | R2625 | 1 | mM | 0.1 | μM |
| U0126 | Cell Signaling | 9903S | 10 | mM | 0-10* | μM |
| | Technologies | | | | | |
| Y-27632 | STEMCELL | 72304 | 10 | mM | 0-10* | μM |
| | Technologies | | | | | |

TABLE E
CD Medium

| | | | Stock | Final |
|---|---|---|---|---|
| Components | Vendor | Cat. no. | concentration | concentration |
| Advanced | Thermo | 12633-012 | — | 99% |
| RPMI 1640 | Scientific | | | (vol/vol) |
| medium | | | | |
| GlutaMAX | Thermo | 35050061 | — | 1% |
| | Scientific | | | (vol/vol) |
| AVP | Sigma | V9879 | 100 μM | 10 nM |
| Aldo | Sigma | A9477 | 100 μM | 10 nM |

Quality control and endpoint analyses (Step 37A-B). In addition to the morphological characteristics described above at each of the stages of differentiation, which were routinely observed throughout the protocol, we characterized key molecular features of the organoids at different timepoints. We routinely performed immunofluorescent staining for quality control on the 2D monolayer cells and the 3D tissues, including on both frozen sections and wholemount samples. The latter was preferable at early stages given the small size and complex morphology. For initial protocol optimization, we ensured high efficiency induction of TBXT at day 1 and PAX2/GATA3 by day 3. From days 11-14, the branching organoids expressed PAX2, GATA3, and CDH1 throughout the epithelia and have RET specifically localized in the tip domains. At day 18 following CD differentiation, AQP2 should be expressed in large numbers of cells within the organoids. In general, we prefer to perform wholemount staining on organoids at days 7-14 since it this method more thoroughly demonstrates their complete structure and morphology (FIG. 16c). At day 18 we have always performed staining on frozen sections, although it is likely they would also be amenable to wholemount staining.

### Detailed Protocol

The following is a detailed description of an exemplary protocol used.

Steps 1-10: Routine Maintenance of hPSCs in Feeder-Free Conditions with mTeSR1 Medium

All maintenance culture experiments described here used mTesR1 media and 6-well plates coated with LDEV-free hESC-qualified Matrigel.

hPSC Maintenance TIMING 20 Minutes

1 Cultures in 6-well plates were fed with daily medium exchanges using 2 ml complete mTesR1.

2 Monitored the growth and quality of the hPSC cultures daily. We routinely passaged cells every 4-5 days once they became ~85-90% confluent and maintained cultures with <5% spontaneous differentiation. If spontaneous differentiation was excessive (as demonstrated in FIG. 13c), differentiated tissues were removed manually using a tungsten needle or pipette tip. Cultures with spontaneous differentiation exceeding 20% were discarded and not used for differentiation.

hPSC Passaging—Timing: 40 Minutes

The starting density of the cells was important to obtain optimal results in the following steps.

3 Before passaging, 6-well plates coated with hESC-qualified Matrigel were prepared or pre-coated plates and medium were warmed to room temperature.

4 The mTeSR1 medium was aspirated, then the cells were washed well with warmed 2 ml DMEM media once and 1 ml of dissociation solution for human ES/iPSCs (Gentle Cell Dissociation Reagent) was added. The cells were incubated for 5 minutes at room temperature.

5 The dissociation solution was aspirated and 1 ml of mTeSR1 added. The colonies were gently detached by gently scraping with a cell scraper.

6 2 ml of additional mTeSR1 was added and the colonies triturated into small clumps by pipetting up and down with a 5-ml serological pipette.

A uniform suspension of colonies approximately 50-200 μm in size was determined to be optimal.

7 In the new plate, the Matrigel solution was aspirated and 1.5 ml/well of fresh mTeSR1 was added.

8 The triturated colonies were passaged into the new plate with fresh medium at desired ratio. We usually used a ratio of 1:6-1:10; for example, plate 0.3-0.5 ml of hPSC clumps/medium (from a total of 3 mL) into each well.

9 The 6-well plates were placed in the 37° C. incubator. The new colonies were distributed evenly by gently shaking the plate back-and-forth and side-to-side several times.

10 The medium was changed every day with 2 ml fresh mTeSR1, and the cells were passaged every 4-5 days.

Steps 11-19: Differentiation Day −1: Plating hPSCs—Timing: 40 Minutes

11 hPSCs were suitable for plating when they have reached ~85% confluence in the 6-well plate, or when they would otherwise be ready for passaging (according to colony morphology in FIG. 13a).

12 A new 24-well plate was coated with hESC-qualified Matrigel or a pre-coated plate was warmed at room temperature for at least 60 minutes.

13 Medium preparation. mTeSR1 was added to a 15-ml conical tube and supplement with ROCK inhibitor Y27632 (10 μM, 1:1,000 dilution). Enough media was prepared to plate cells using 0.5 ml/well with at least 1 ml extra for resuspending cells and pipetting error.

14 Dissociate cells. The mTeSR1 media was aspirated from one well of hPSCs in a 6-well plate, then the cells were washed once with 2 ml DMEM. 1 ml Accutase was added, then the cells were placed in an incubator at 37° C. and 5% CO₂ for 7 minutes. At the end of incubation, the majority of the cells detached from the plate with gentle shaking or tapping. If >30% cells remain attached at this point, the plate was placed back in the incubator for an additional 3 minutes.

15 Following incubation, 2 mL DMEM was add to each well then pipetted up-and-down several times to completely detach and dissociate the cells into a single cell suspension.

16 The cell suspension from each well was collected into a 15-ml conical tube, and the tube was centrifuged at 300×g at room temperature for 3 minutes.

17 Following centrifugation, the supernatant was aspirate and the cells resuspended in 0.5 ml mTeSR+Y-27632 (10 μM, 1:1,000 dilution).

18 The cells were counted using a hemacytometer. Enough cells were transferred to the 15 ml tube of mTeSR 1+Y-27632 (10 μM, 1:1,000 dilution) to create a suspension with a concentration of 6×10⁴ cells/ml.

19 Plate cells. The Matrigel solution was aspirated from the coated 24-well plates and 0.5 ml cell suspension was added per well. The 24-well plates were placed in the 37° C. incubator, distribute cells by gently shaking the plate, and incubate overnight.

### Steps 20-22: Differentiation Day 0: Induction of Primitive Streak Progenitor Cells-Timing: 15 Minutes

20 At approximately 24 hours after plating, cells were about 20-30% confluent in the 24-well plate. FIG. 13b shows representative morphology and density of cultures at day 0.

If the starting cell density was too low or too high (<15% or >40%), the differentiation efficiency was likely to be negatively affected.

21 mTeSR1 was aspirated and 500 μl/well of DO Medium (Table A) was added. The cells were then incubated overnight.

22 We monitored morphology in the cultures starting 24 hours after addition of DO Medium. At this point, there was typically a high proportion of colony-like clusters remaining, as shown in FIG. 14. We continued to observe the cells for another 4-6 hours, during which time the colonies dispersed such that the dominant cell population comprised single cells with a mesenchymal phenotype. We only proceeded to the next step once this morphology was observed, which typically occurs 27-30 hours after addition of DO Medium.

The differentiation efficiency of pronephric IM cells was sensitive to this step. The optimal timing for this step was usually 25-30 hours, but might alter for different cell line. We closely monitored the cell morphology in all differentiation experiments, with the ideal incubation period being determined as the earliest time at which colony morphology was no longer obvious. FIG. 14 shows that the differentiation efficiency, assayed by the co-expression of PAX2 and GATA3 at day 3, was dramatically reduced when IM Medium is added either too early or too late.

### Steps 23-25: Differentiation Day 1: IM Specification-Timing: 30 Minutes

23 Media was aspirated and 750 μl/well of IM Medium (Table B) was added. The cells were then incubated for ~24 hours.

24 After ~24 hours, the media was aspirated and replaced with 750 μl/well fresh IM Medium.

25 After another 24 hours, the cells were a dense monolayer as shown in FIG. 14.

The high efficiency of IM induction was essential for the differentiation. We routinely monitored PAX2 and GATA3 positivity by IF staining on day 3 as a quality control checkpoint.

### Steps 26: Differentiation Day 3: Spheroid Formation-Timing: 60 Minutes

26 At day 3, IM cells were dissociated and aggregated into 3D spheroids. The aggregation was performed in either of two formats: 96-well low-attachment plates (Option A) or patterned microwell AggreWell plates (Option B)

### (A) Induction of 3D Spheroids in 96-Well, Round-Bottom Plates

(i) ND Medium was prepared for days 3-5 as shown in Table C, making enough to plate the desired number of wells (200 μl/well of 96-well plate).

(ii) The differentiation medium was aspirated, and the cells washed with DMEM media and add 250 μl/well Accutase. The cells were incubated at 37° C. for 7 minutes.

(iii) After incubation, 750 μl/well DMEM was added to the wells and pipeted using P1000 several times to completely detach the cells and create a single cell suspension.

(iv) The cell suspension was collected into a 15-ml conical tube, and the tubes were centrifuged at 300×g at room temperature for 4 minutes.

(v) The supernatant was aspirated and the cells resuspended in prepared ND Medium at a concentration of 1.0-1.5×10⁵ cells/ml.

(vi) A 200 μl/well cell suspension was plated into 96-well, round-bottom, ultra-low-attachment plates.

(vii) The plates were centrifuged at 100×g for 15 seconds to form multiple small aggregates in each well. The cells were cultured at 37° C. for 2 days without changing medium.

To observe spheroid formation under the microscope, we moved and handled the plates very gently. Agitation was more likely to cause individual spheroids to aggregate together into a large structure.

(viii) On day 5, a half-medium change was performed by gently aspirating 100 μl medium and adding 100 μl ND Medium for days 5-7 containing GDNF. The plates were placed back in the 37° incubator and cultured for an additional 2 days.

(ix) Between days 5-7, spheroids segregated into two lineages as shown in FIG. 15A, where the larger cell population comprised ND progenitor cells that co-express PAX2 and GATA3.

### (B) Induction of 3D Spheroids in AggreWell-400 Plates

(i) ND Medium was prepared for days 3-5 as shown in Table C, making enough to plate desired number of wells (2 ml/well in AggeWell-400 24-well plate).

(ii) The AggreWell-400 plate was removed from its packaging. 500 μl/well of Anti-Adherence Rinsing Solution was added into the wells that would be used. The unused wells in the plate were stored for use in other experiments. The plate was centrifuged at 1,300×g for 5 minutes at room temperature.

(iii) Following centrifugation, the rinse solution was aspirated from the wells and rinsed once with 500 μl DMEM. 1 ml ND Medium was added to each well.

(iv) The differentiation medium was aspirated from day 3 cells, then the cells were washed with DMEM media and 250 μl/well Accutase was added. The cells were incubated at 37° C. for 7 minutes.

(v) After incubation, 750 μl/well DMEM was added to the wells and pipetted using P1000 several times to completely detach cells and create a single cell suspension.

(vi) The cell suspension was collected into a 15-ml conical tube. (Typically 1 well of the day 3 cells in a 24 well plate is enough to seed 2 wells of the AggreWell plate) The tubes were centrifuged at 300×g at room temperature for 4 minutes.

(vii) The supernatant was aspirated and the cells resuspended in prepared ND Medium at a concentration of 1.2×10⁶ cells/ml.

(viii) 1 ml of the cell suspension was added to each well of the AggreWell plate (final volume was 2 ml/well).

(ix) The plate was centrifuged at 100×g for 3 minutes to pellet the cells into the microwells. The cells were cultured at 37° C. for 2 days without changing medium.

To observe spheroid formation under the microscope, we gently moved and handled the plates. Agitation was more likely to cause individual spheroids to aggregate together into a large structure.

(x) On day 5, a half-medium change was performed by gently aspirating 1 ml medium and adding 1 ml ND Medium for days 5-7 containing GDNF. The plate was placed back in the incubator and cultured for an additional 2 days.

(xi) Between days 5-7, spheroids segregated into two lineages as shown in FIG. 15b-d, where the larger cell population comprises ND progenitor cells that co-express PAX2 and GATA3.

In most cases, there was a small degree of clumping of individual spheroids into larger aggregates, but this was acceptable and did not impact the efficiency or downstream steps of differentiation. However all handling of the plates and media changes was performed very slowly and gently to minimize this effect and avoid mass clumping of spheroids into huge aggregations.

### Steps 27-34: Differentiation Day 7: Plating Spheroids in 3D Matrigel-Timing: 120 Minutes

27 At least 30 minutes before moving to the next step, aliquots of thawed Matrigel Matrix was placed at 4° C.

28 Spheroids were collected into 1.5 ml microcentrifuge tubes using a P1000 pipette. Each tube contained enough spheroids to plate in Matrigel into 12 wells of a 24-well plate.
A. From 96-well plates, all spheroids were combined from 12-18 wells into a single tube.
B. From an AggreWell-400 plate, we first pipetted multiple times using a P1000 pipette to lift spheroids out of their microwells and generate a spheroid suspension. About 750 μl of the suspension was transferred into each microcentrifuge tube.

29 These tubes were placed upright in a tube rack for ~10-15 minutes to allow the spheroids to settle by gravity to the bottom of the tube, then gently aspirated to leave the spheroids remaining in a minimal volume of medium, e.g., less than 20 μl.

30 600 μl/tube cold Matrigel Matrix from Step 27 was added into a microcentrifuge tube and pipetted multiple times to evenly suspend and distribute the spheroids in the matrix suspension.

31 Using a P200 with wide-bore tip, ~45 μl/well of spheroid/matrix suspension was pipetted into a 3D dome in the center of each well of Nunclon delta surface 24-well tissue culture plate³⁴. The Matrigel suspension was enough to plate 12 wells.

The Matrigel Matrix will gel rather quickly at room temperature, so it was imperative to work quickly during plating. Steps 29-31 were performed separately for each microcentrifuge tube of spheroids, as trying to do them in parallel increased the likelihood of Matrigel solidifying prior to plating.

Using Nunclon delta surface tissue culture plates was essential for formation of a stable Matrigel dome^{34.}

32 The plates were placed in the 37° C. incubator for 45-60 minutes to allow the Matrigel to solidify after finishing embedding all spheroids.

33 500 μl/well UB Medium (Table D) was added carefully to the side of each well of the plate. For differentiation of new cell lines, the concentrations of U0126 (0-10 μM) and Y-27632 (0-10 μM) in the UB Medium could be altered to promote the most robust branching morphology (demonstrated in FIG. 17).

(i) The UB Medium was prepared with at least three different concentrations of U0126 (0, 2, and 5 μM), each with and without ROCK inhibitor Y-27632 (10 μM). The medium for each condition was added to 2-3 wells of freshly plated spheroids.

(ii) The UB organoid growth was monitored for 3-4 days to identify the condition that promoted the optimal branching morphology.

34 The organoids were cultured at 37° C. for 7 days, replacing with fresh 500 μl/well UB Medium every 3-4 days. UB organoids exhibited branching morphogenesis during this period (FIG. 16a).

### Steps 35-36: Differentiation Day 14: Inducing CD Differentiation in Organoids—Timing: 20 Minutes

35 To stimulate principal cell differentiation, the UB Medium was aspirated and 500 μl/well CD Medium (Table E) was added.

36 The organoids were cultured at 37° C. for 4 days. Media change was not necessary in this step.

We cultured the organoids for 3-4 days to induce the formation of CD cell types. This differentiation process also worked with comparable efficiency when started at slightly later stages.

### Step 37: Quality Control and Endpoint Analyses

UB organoids were harvested and analyzed at the specific stage of interest. Developmental markers of the UB lineage were expressed through at least day 14, while genes associated with differentiated CD fates were expressed between days 16-18.

37 We routinely performed both quality control and end-point analyses using immunofluorescent staining, using either (A) direct staining of fixed cells (days 1 and 3), (B) frozen sectioning and staining of 3D UB/CD organoids, or (C) whole-mount staining of 3D UB/CD organoids.

### (A) Quality Control Analysis for Monitoring Efficient Induction of Either TBXT-Positive Mesendoderm Progenitors (Day 1) or PAX2/GATA3-Positive Pronephric IM Progenitors—Timing 2 Days

(i) To immunostain cells directly in the 24-well plate, the differentiation medium was aspirated and wells washed once with PBS.

(ii) PBS was aspirated, the 250 μl/well 4% (wt/vol) PFA was added to the cells, and incubated at room temperature for 45 minutes.

(iii) The PFA was removed and discarded, and 0.5 ml/well PBS added, and the plate was place on an orbital shaker with gentle shaking for 5 minutes. This was repeated at least three times to wash the cells.

(iv) PBS was aspirated following the final wash, then the cells were permabilized and blocked by adding 250 μl fresh immunostaining blocking buffer, then incubated for 30 minutes at room temperature.

(v) The immunostaining blocking buffer was aspirated and ~250 μl fresh immunostaining blocking buffer containing primary antibodies was added. The cells were incubated at 4° C. overnight on a platform rocker with gentle rocking.

(vi) The antibody solution was aspirated and the slides washed with PBS three times.

(vii) 250 μl immunostaining blocking buffer containing secondary antibodies and nuclear stain such as DAPI was added, then the plate was wrapped in aluminum foil and incubated at room temperature for one hour on an orbital shaker.

(viii) The antibody solution was aspirated and the cells washed with PBS three times. After the final wash, ~250 μl PBS was left in the wells.

(ix) The cells and staining were imaged directly in the plate using an inverted fluorescent microscope.

### (B) End-Point Analysis for 3D UB/CD Organoids by Frozen Sectioning and Staining—Timing: 3 Days

(i) The differentiation medium was aspirated and wells washed once with 1 ml PBS

(ii) PBS was aspirated, 500 μl/well 4% (wt/vol) PFA in PBS was added to fix the organoids in the matrix. The organoids were incubated at room temperature for one hour.

(iii) The PFA was aspirated, add 1 ml/well PBS, and placed on a rocker with gentle rocking for 5 minutes. This was repeated at least three times to thoroughly wash the organoids.

(iv) PBS was aspirated following the final wash, then the organoids pipetted several times using a wide-bore tip to break the Matrigel and separate the organoids.

(v) The organoids were transferred to the center of a cryomold and as much PBS was aspirated from the mold as possible. The OCT compound was added to fill the cryomolds and allowed to sit at room temperature for 20-30 minutes.

(vi) The samples were frozen in a dry ice/ethanol bath for 5-10 minutes until the OCT compound completely solidified, and the blocks placed in −80° C. freezer overnight. To prepare the bath, we crushed dry ice with a hammer into small (<1 cm) pellets in a Styrofoam container and then added ethanol (100%) to fill to just below the level of the dry ice.

(vii) Frozen sections of 7 μm thickness were cut using a cryostat, and then mounted on glass slides. Slides were stored in a slidebox at −80° C. before staining.

(viii) For staining, slides were washed once in PBS for 10 minutes. After washing, tissue was circled on the slide using a hydrophobic pen and the tissue covered completely with immunostaining blocking buffer.

(ix) The slides were incubated at room temperature for one hour for blocking.

(x) The immunostaining blocking buffer was aspirated and ~100 μl fresh immunostaining blocking buffer containing primary antibodies was added. The slides were incubated at 4° C. overnight.

(xi) The antibody solution was aspirated and the slides washed with PBS three times.

(xii) ~100 μl immunostaining blocking buffer containing secondary antibodies and nuclear stain such as DAPI was added. The slides were incubated at room temperature for one hour in a dark box.

(xiii) The antibody solution was aspirated and the slides washed with PBS three times.

(xiv) The slides were mounted with 2-3 drops of Fluoromount G mounting medium and a coverslip added.

(xv) The slides were air-dried at room temperature overnight. Imaging was performed using a widefield fluorescent microscope or a confocal microscope.

### (C) End-Point Analysis for 3D UB/CD Organoids by Whole-Mount Staining-TIMING 2 Days

(i) The differentiation medium was aspirated and the wells washed once with PBS.

(ii) PBS was aspirated, 500 μl/well 4% (wt/vol) PFA in PBS was added to fix the organoids in the matrix. The organoids were incubated at room temperature for one hour.

(iii) The PFA was aspirated, 1 ml/well PBS added, and placed on a rocker with gentle rocking for 5 minutes. This was repeated at least three times to thoroughly wash the organoids.

(iv) PBS was aspirated following the final wash, then the organoids were pipetted several times using a wide-bore tip to break Matrigel and separate the organoids.

(v) Individual or small groups of organoids were transferred into a well of a 96-well, round-bottom plate for staining.

Given the small size of the UB organoids, we performed aspiration in the steps below using a P200 pipet under a stereomicroscope to avoid losing excessive amount of tissue.

(vi) As much PBS as possible was aspirated without losing organoids. 50 μl/well immunostaining blocking buffer was added and incubated at room temperature for one hour.

(vii) The immunostaining blocking buffer was aspirated and ~50 μl fresh immunostaining blocking buffer containing primary antibodies was added and the organoids were incubated at 4° C. overnight.

(viii) The antibody solution was aspirated and the organoids washed with PBS three times.

(ix) The PBS was aspirated and ~50 μl immunostaining blocking buffer containing secondary antibodies and nuclear stain such as DAPI was added.

(x) The plate was wrapped with aluminum foil and the organoids were incubated at room temperature for two hours.

(xi) The antibody solution was aspirated and the organoids were washed with PBS three times.

(xii) The organoids were transferred to a glass slide and PBS was aspirated. The tissue was mounted with one drop of Fluoromount G mounting medium and a coverslip added to the slide. Alternatively, if using an inverted confocal microscope, the organoids were directly imaged in the well without mounting on a slide.

(xiii) Imaging was performed using a widefield fluorescent microscope or a confocal microscope. For tissue at days 7-14, we mounted the organoids flat on a glass slide with a coverslip to enable visualization of the majority of the organoid in a single plane.

### Timing

Steps 1-10, maintenance of hPSCs in feeder-free culture with mTeSR1 media: 4 days
Steps 11-19, preparation of hPSCs for differentiation: one day
Steps 20-25, differentiation of hPSCs into pronephric intermediate mesoderm cells: 3 days
Step 26A-B, induction of 3D nephric duct spheroids: 4 days
Steps 27-34, generation of branching UB organoids: 7 days
Steps 35-36, differentiation of CD organoids: 4 days
Step 37, end-point analysis: 2-3 days

### Results

With careful attention to maintenance of high quality hPSC cultures and optimization of timing of mesendodermal specification, this protocol yielded pronephric IM progenitors at high efficiency (at least ~90%) on day 3 of differentiation (FIG. 14). From this stage, 3D aggregation of the progenitors generated ND spheroids with remarkable consistency in either 96-well plates or AggreWell-400 microwell plates. Typically, 100% of the spheroids exhibited morphological and molecular segregation into ND and stromal domains (FIG. 15a-d), and the observation of this phenomenon was a reliable surrogate indicator of the success of the differentiation procedure. Overall, these early stages were associated with high rates of proliferation, such that starting with only several wells of a 24-well plate was sufficient to produce thousands of individual ND spheroids and UB organoids.

Embedding ND spheroids in Matrigel for 3D culture promoted growth and branching of UB organoids for a duration of up to one week (FIG. 16a). Initially there was rapid growth with the formation of numerous stalks emanating from each sphere, which then underwent branching via bifurcation at the tips. Analysis of UB organoids between days 11-14 showed ubiquitous expression of CDH1 and UB transcription factors including PAX2, GATA3, SOX9, and LHX1 (FIG. 16b). Further, the tip domains specifically expressed RET, a marker of the progenitor cells that are receptive to niche signals such as GDNF. At the end of the differentiation after inducing CD induction, AQP2-expressing principal cells were abundant throughout the epithelium (FIG. 16c).

The success of these methods and obtaining UB organoids with desired morphology was dependent upon optimizing the efficiency of induction of pronephric IM progenitors at day 3. Confirming differentiation outcomes at day 1 (staining for TBXT) and day 3 (staining for PAX2 and GATA3) prior to proceeding with subsequent stages was optimal, since low efficiency (<80%) cultures result in failed formation of ND spheroids and UB organoids.

### REFERENCES

1. Kim, J., Koo, B. K. & Knoblich, J. A. Human organoids: model systems for human biology and medicine. Nat Rev Mol Cell Biol 21, 571-584 (2020).
2. Lehmann, R. et al. Human organoids: a new dimension in cell biology. Mol Biol Cell 30, 1129-1137 (2019).
3. McMahon, A. P. Development of the Mammalian Kidney. Curr Top Dev Biol 117, 31-64 (2016).
4. Takasato, M. et al. Kidney organoids from human iPS cells contain multiple lineages and model human nephrogenesis. Nature 526, 564-568 (2015).
5. Morizane, R. et al. Nephron organoids derived from human pluripotent stem cells model kidney development and injury. Nat Biotechnol 33, 1193-1200 (2015).
6. Taguchi, A. & Nishinakamura, R. Higher-Order Kidney Organogenesis from Pluripotent Stem Cells. Cell Stem Cell 21, 730-746 e736 (2017).
7. Kuraoka, S. et al. PKD1-Dependent Renal Cystogenesis in Human Induced Pluripotent Stem Cell-Derived Ureteric Bud/Collecting Duct Organoids. J Am Soc Nephrol 31, 2355-2371 (2020).
8. Zeng, Z. et al. Generation of patterned kidney organoids that recapitulate the adult kidney collecting duct system from expandable ureteric bud progenitors. Nat Commun 12, 3641 (2021).
9. Mae, S. I. et al. Expansion of Human iPSC-Derived Ureteric Bud Organoids with Repeated Branching Potential. Cell Rep 32, 107963 (2020).
10. Howden, S. E. et al. Plasticity of distal nephron epithelia from human kidney organoids enables the induction of ureteric tip and stalk. Cell Stem Cell 28, 671-684 e676 (2021).
11. Bens, M. et al. Corticosteroid-dependent sodium transport in a novel immortalized mouse collecting duct principal cell line. J Am Soc Nephrol 10, 923-934 (1999).
12. Prie, D. et al. Role of adenosine on glucagon-induced CAMP in a human cortical collecting duct cell line. Kidney Int 47, 1310-1318 (1995).
13. Fejes-Toth, G. & Naray-Fejes-Toth, A. Differentiation of renal beta-intercalated cells to alpha-intercalated and principal cells in culture. Proc Natl Acad Sci USA 89, 5487-5491 (1992).
14. Qiao, J., Sakurai, H. & Nigam, S. K. Branching morphogenesis independent of mesenchymal-epithelial contact in the developing kidney. Proc Natl Acad Sci USA 96, 7330-7335 (1999).
15. Grote, D., Souabni, A., Busslinger, M. & Bouchard, M. Pax 2/8-regulated Gata 3 expression is necessary for morphogenesis and guidance of the nephric duct in the developing kidney. Development 133, 53-61 (2006).
16. Barak, H., Rosenfelder, L., Schultheiss, T. M. & Reshef, R. Cell fate specification along the anterior-posterior axis of the intermediate mesoderm. Dev Dyn 232, 901-914 (2005).
17. Loh, K. M. et al. Mapping the Pairwise Choices Leading from Pluripotency to Human Bone, Heart, and Other Mesoderm Cell Types. Cell 166, 451-467 (2016).
18. Ornitz, D. M. & Itoh, N. The Fibroblast Growth Factor signaling pathway. Wiley Interdiscip Rev Dev Biol 4, 215-266 (2015).
19. Perantoni, A. O. et al. Inactivation of FGF8 in early mesoderm reveals an essential role in kidney development. Development 132, 3859-3871 (2005).
20. Warga, R. M., Mueller, R. L., Ho, R. K. & Kane, D. A. Zebrafish Tbx16 regulates intermediate mesoderm cell fate by attenuating Fgf activity. Dev Biol 383, 75-89 (2013).
21. Mae, S. I. et al. Generation of branching ureteric bud tissues from human pluripotent stem cells. Biochem Biophys Res Commun 495, 954-961 (2018).
22. Bohnenpoll, T. et al. Tbx18 expression demarcates multipotent precursor populations in the developing urogenital system but is exclusively required within the ureteric mesenchymal lineage to suppress a renal stromal fate. Dev Biol 380, 25-36 (2013).
23. Attia, L., Schneider, J., Yelin, R. & Schultheiss, T. M. Collective cell migration of the nephric duct requires FGF signaling. Dev Dyn 244, 157-167 (2015).
24. Atsuta, Y. & Takahashi, Y. FGF8 coordinates tissue elongation and cell epithelialization during early kidney tubulogenesis. Development 142, 2329-2337 (2015).
25. Sanchez-Ferras, O. et al. A coordinated progression of progenitor cell states initiates urinary tract development. Nat Commun 12, 2627 (2021).
26. Pohl, M., Stuart, R. O., Sakurai, H. & Nigam, S. K. Branching morphogenesis during kidney development. Annu Rev Physiol 62, 595-620 (2000).
27. Yuri, S., Nishikawa, M., Yanagawa, N., Jo, O.D. & Yanagawa, N. In Vitro Propagation and Branching Morphogenesis from Single Ureteric Bud Cells. Stem Cell Reports 8, 401-416 (2017).
28. Vega, Q. C., Worby, C. A., Lechner, M. S., Dixon, J. E. & Dressler, G. R. Glial cell line-derived neurotrophic factor activates the receptor tyrosine kinase RET and promotes kidney morphogenesis. Proc Natl Acad Sci USA 93, 10657-10661 (1996).
29. Michos, O. et al. Kidney development in the absence of Gdnf and Spry 1 requires Fgf10. PLOS Genet 6, e1000809 (2010).
30. Lu, B. C. et al. Etv4 and Etv5 are required downstream of GDNF and Ret for kidney branching morphogenesis. Nat Genet 41, 1295-1302 (2009).
31. Bush, K. T. et al. TGF-beta superfamily members modulate growth, branching, shaping, and patterning of the ureteric bud. Dev Biol 266, 285-298 (2004).
32. Maeshima, A., Vaughn, D. A., Choi, Y. & Nigam, S. K. Activin A is an endogenous inhibitor of ureteric bud outgrowth from the Wolffian duct. Dev Biol 295, 473-485 (2006).
33. Michos, O. et al. Reduction of BMP4 activity by gremlin 1 enables ureteric bud outgrowth and GDNF/WNT11 feedback signalling during kidney branching morphogenesis. Development 134, 2397-2405 (2007).
34. Chi, X. et al. Ret-dependent cell rearrangements in the Wolffian duct epithelium initiate ureteric bud morphogenesis. Dev Cell 17, 199-209 (2009).
35. Costantini, F. GDNF/Ret signaling and renal branching morphogenesis: From mesenchymal signals to epithelial cell behaviors. Organogenesis 6, 252-262 (2010).
36. Tsujimoto, H. et al. A Modular Differentiation System Maps Multiple Human Kidney Lineages from Pluripotent Stem Cells. Cell Rep 31, 107476 (2020).
37. Uchimura, K., Wu, H., Yoshimura, Y. & Humphreys, B. D. Human Pluripotent Stem Cell-Derived Kidney Organoids with Improved Collecting Duct Maturation and Injury Modeling. Cell Rep 33, 108514 (2020).
38. Unbekandt, M. & Davies, J. A. Dissociation of embryonic kidneys followed by reaggregation allows the formation of renal tissues. Kidney Int 77, 407-416 (2010).
39. Hao, Y. et al. Integrated analysis of multimodal single-cell data. Cell 184, 3573-3587 e3529 (2021).
40. Lake, B. B. et al. An atlas of healthy and injured cell states and niches in the human kidney. bioRxiv, 2021.2007.2028.454201 (2021).
41. Kleyman, T. R. & Cragoe, E. J., Jr. Amiloride and its analogs as tools in the study of ion transport. J Membr Biol 105, 1-21 (1988).
42. Chen, L., Chou, C. L. & Knepper, M. A. A Comprehensive Map of mRNAs and Their Isoforms across All 14 Renal Tubule Segments of Mouse. J Am Soc Nephrol (2021).
43. Werth, M. et al. Transcription factor TFCP2L1 patterns cells in the mouse kidney collecting ducts. Elife 6 (2017).
44. Park, J. et al. Single-cell transcriptomics of the mouse kidney reveals potential cellular targets of kidney disease. Science 360, 758-763 (2018).
45. Blomqvist, S. R. et al. Distal renal tubular acidosis in mice that lack the forkhead transcription factor Foxi1. J Clin Invest 113, 1560-1570 (2004).
46. Toka, H. R., Toka, O., Hariri, A. & Nguyen, H. T. Congenital anomalies of kidney and urinary tract. Semin Nephrol 30, 374-386 (2010).
47. Kuure, S. & Sariola, H. Mouse Models of Congenital Kidney Anomalies. Adv Exp Med Biol 1236, 109-136 (2020).
48. Shah, M. M. et al. The instructive role of metanephric mesenchyme in ureteric bud patterning, sculpting, and maturation and its potential ability to buffer ureteric bud branching defects. Am J Physiol Renal Physiol 297, F1330-1341 (2009).
49. Schwartz, G. J. et al. Acid incubation reverses the polarity of intercalated cell transporters, an effect mediated by hensin. J Clin Invest 109, 89-99 (2002).
50. Schwartz, G. J., Barasch, J. & Al-Awqati, Q. Plasticity of functional epithelial polarity. Nature 318, 368-371 (1985).
51. Christensen, B. M. et al. Changes in cellular composition of kidney collecting duct cells in rats with lithium-induced NDI. Am J Physiol Cell Physiol 286, C952-964 (2004).
52. Davies, J. A., Unbekandt, M., Ineson, J., Lusis, M. & Little, M. H. Dissociation of embryonic kidney followed by re-aggregation as a method for chimeric analysis. Methods Mol Biol 886, 135-146 (2012).
53. Przepiorski, A. et al. A Simple Bioreactor-Based Method to Generate Kidney Organoids from Pluripotent Stem Cells. Stem Cell Reports 11, 470-484 (2018).
54. Morizane, R. & Bonventre, J. V. Generation of nephron progenitor cells and kidney organoids from human pluripotent stem cells. Nat Protoc 12, 195-207 (2017).
55. Pepicelli, C. V., Kispert, A., Rowitch, D. H. & McMahon, A. P. GDNF induces branching and increased cell proliferation in the ureter of the mouse. Dev Biol 192, 193-198 (1997).

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A method for providing an artificial branching ureteric bud (UB) organoid from a population of mammalian pluripotent stem cells (PSC), the method comprising:
(i) culturing the PSC to induce formation of a population of cells comprising at least 90%, 91%, 92%, 93%, or 94% TBXT-positive mesendodermal progenitor cells (MPCs);
(ii) culturing the population of MPCs in a serum-free growth medium in the presence of retinoic acid (RA) or an analog thereof, fibroblast growth factor 2 (FGF2), an inhibitor of BMP, and an inhibitor of TGFβ signaling for about 48 hours to induce formation of a population of cells comprising at least 85% or 86% PAX2-positive/GATA3-positive/LHX1-positive intermediate mesoderm (IM) progenitor cells (IMPCs) for about 28-32 hours;
(iii) promoting aggregation of the IMPCs into spheroids, preferably spheroids having a diameter between 50-200 μm;
(iv) culturing the IMPC spheroids in the presence of a serum-free growth medium containing only RA, or an analog thereof, and FGF9 for about two days to form spheroids comprising GATA3-positive/PAX8-positive cells;
(v) culturing the nephric duct spheroids in a serum-free growth medium in the presence of RA or an analog thereof, and glial cell line-derived neurotrophic factor (GDNF) for about two days to form a population of nephric duct spheroids comprising GATA3-positive/PAX2 positive/RET-positive cells;
(vi) embedding the nephric duct spheroids in a natural or synthetic hydrogel scaffold, preferably comprising a natural extracellular matrix (ECM), and
(vii) culturing in the presence of media comprising FGF10, GDNF, a Wnt agonist, a BMP inhibitor, a TGF-β type I inhibitor, RA, and a MEK inhibitor, and optionally a ROCK inhibitor, for 4-10 days; thereby providing an artificial UB organoid.

2. The method of claim 1, wherein the PSC is a human PSC (hPSC).

3. The method of claim 1, wherein step (i) comprises culturing the PSC in the presence of a WNT agonist, preferably a GSK3β inhibitor, optionally CHIR99021 or BIO; FGF2; BMP4; and TGFβ, preferably Activin A.

4. The method of claim 1, wherein step (ii) comprises culturing the population of MPCs in the presence of retinoic acid (RA) or TTNBP; FGF2; an inhibitor of BMP selected from LDN193189, DMH-1, or dorsomorphin; and an inhibitor of TGFβ signalling selected from A8301 or SB-431542.

5. The method of claim 1, wherein promoting aggregation of the IMPCs into spheroids in step (iii) comprises plating the cells at low density on a low attachment substrate or on a patterned microwell plate.

6. The method of claim 1, wherein step (iv) comprises culturing the IMPC spheroids in the presence of a serum-free growth medium containing only RA and human FGF9.

7. The method of claim 1, wherein step (v) comprises culturing the nephric duct spheroids in a serum-free growth medium in the presence of RA and human GDNF.

8. The method of claim 1, wherein the natural or synthetic hydrogel scaffold in step (vi) comprises natural extracellular matrix (ECM).

9. The method of claim 1, wherein step (vii) comprises culturing in the presence of media comprising human FGF10; human GDNF; a Wnt agonist selected from CHIR99021 or BIO; an inhibitor of BMP selected from LDN193189, DMH-1, or dorsomorphin; an inhibitor of TGFβ signalling selected from A8301 or SB-431542; RA; and MEK inhibitor U0126, and optionally ROCK inhibitor Y-27632.

10. The method of claim 1, further comprising:
(viii) incubating the artificial UB organoid in media comprising arginine vasopressin (AVP) and aldosterone (Aldo) for about 3-4 days, to induce formation of collecting duct (CD) organoids comprising AQP2-positive principal cells (PCs).

11. The method of claim 10, further comprising:
(ix) inducing FOXI1 expression in the CD organoids for about four days, to induce differentiation of ATP6V1B1-positive intercalated cells (ICs).

12. The method of claim 1, which does not comprise cell sorting or purification.

13. The method of claim 1, further comprising dissociating the cells of the UB or CD organoid.

14. An artificial UB organoid obtained by the method of claim 1.

15. An isolated cell obtained from the artificial UB organoid of claim 14, optionally a PAX2-positive/GATA3-positive/RET-positive cell.

16. An artificial CD organoid obtained by the method of claim 10.

17. An isolated cell obtained from the artificial CD organoid of claim 16, optionally a principal cell (PC) or an intercalated cell (IC).

18. A method of screening a test compound, the method comprising performing the method of claim 1 for generation of UB or CD organoids in the presence and absence of a test compound, and determining an effect of the test compound on development of the UB or CD organoids.

19. A method of screening a test compound, the method comprising:
providing a UB organoid obtained by the method of claim 1, or a CD organoid obtained by the method of claim 1, further comprising (viii) incubating the artificial UB organoid in media comprising arginine vasopressin (AVP) and aldosterone (Aldo) for about 3-4 days, to induce formation of collecting duct (CD) organoids comprising AQP2-positive principal cells (PCs), and optionally (ix) inducing FOXI1 expression in the CD organoids for about four days, to induce differentiation of ATP6V1B1-positive intercalated cells (ICs);
incubating the UB organoid or CD organoid in the presence and absence of a test compound; and
detecting an effect of the test compound on a parameter of the organoid.

20. The method of claim 19, wherein detecting an effect of the test compound on a parameter comprises measuring organoid function or expression of a selected marker.
